(19) United States
(12) Patent Application Publication
(10) Pub. No.: US 2022/0289842 A1
(43) Pub. Date: Sep. 15, 2022

(54) CHIMERIC INHIBITORY RECEPTOR

(71) Applicant: Senti Biosciences, Inc., South San Francisco, CA (US)

(72) Inventors: Russell Morrison Gordley, San Francisco, CA (US); Nicholas Frankel, South San Francisco, CA (US)

(21) Appl. No.: 17/636,301

(22) PCT Filed: Aug. 20, 2020
(86) PCT No.: PCT/US2020/047274

§ 371 Date: Feb. 17, 2022

Related U.S. Application Data
(60) Provisional application No. 62/889,324, filed on Aug. 20, 2019.

(51) Int. Cl. C07K 16/28; C07K 14/705; C12N 9/12; A61K 35/17

(52) U.S. Cl. CPC C07K 16/2803; C07K 14/70503; C12Y 207/10002; C12Y 301/03048; C12N 9/12; A61K 35/17; C07K 2317/622; C07K 2319/03; C07K 2317/55

(57) ABSTRACT

Provided herein are chimeric inhibitory receptor constructs and compositions, and cells comprising the same. Also provided are methods of using chimeric inhibitory receptor constructs and compositions, and cells comprising the same.

### CROSS REFERENCE TO RELATED APPLICATIONS

This application claims the benefit of U.S. Provisional Application No. 62/889,324, filed Aug. 20, 2019, which is hereby incorporated by reference in its entirety.

### SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### BACKGROUND

Chimeric antigen receptors (CARs) enable targeted in vivo activation of immunomodulatory cells, such as T cells. These recombinant membrane receptors have an antigen-binding domain and one or more signaling domains (e.g., T cell activation domains). These special receptors allow the T cells to recognize a specific protein antigen on tumor cells and induce T cell activation and signaling pathways. Recent results of clinical trials with chimeric receptor-expressing T cells have provided compelling support of their utility as agents for cancer immunotherapy. However, despite these promising results, a number of side effects associated the CAR T-cell therapeutics were identified, raising significant safety concerns. One side effect is “on-target but off-tissue” adverse events from TCR and CAR engineered T cells, in which a CAR T cell binds to its ligand outside of the target tumor tissue and induces an immune response. Therefore, the ability to identify appropriate CAR targets is important for effectively targeting and treating the tumor without damaging normal cells that express the same target antigen. The ability to regulate an appropriate response to targets and reduce off-target side effects is important in other immune receptor systems as well, such as TCRs, engineered TCRs, and chimeric TCRs.

Inhibitory chimeric antigen receptors (also known as iCARs) are protein constructions that inhibit or reduce immunomodulatory cell activity after binding their cognate ligands on a target cell. Current iCAR designs leverage PD-1 intracellular domains for inhibition, but have proven difficult to reproduce. Thus, alternative inhibitory domains for use in iCARs are needed. Appropriate inhibitory domains, strategies, and constructs for immune receptor systems are also needed.

### SUMMARY

Provided herein, in some aspects, are chimeric inhibitory receptors that include: an extracellular ligand binding domain; a membrane localization domain that includes a transmembrane domain; and an enzymatic inhibitory domain that inhibits immune receptor activation when proximal to an immune receptor.

Provided herein, in other aspects, are nucleic acids encoding at least one chimeric inhibitory receptor of the present disclosure. In some embodiments, the nucleic acid encoding the at least one chimeric inhibitory receptor is a vector.

In other aspects, genetically engineered cells are provided including a nucleic acid, such as a vector, encoding at least one chimeric receptor of the present disclosure or that express a chimeric inhibitory receptor of the present disclosure. In some aspects, genetically engineered cells expressing a chimeric inhibitory receptor are provided, wherein the chimeric inhibitory receptor includes: an extracellular ligand binding domain; a membrane localization domain, wherein the membrane localization domain comprises a transmembrane domain; and an enzymatic inhibitory domain, wherein the inhibitory domain inhibits immune receptor activation when proximal to an immune receptor.

In still other aspects, methods are provided for inhibiting immune receptor activation in genetically engineered cells of the present disclosure.

In yet other aspects, methods are provided for utilizing genetically engineered cells or pharmaceutical compositions of the present disclosure to reduce an immune response and/or treat an autoimmune disease.

In other aspects, pharmaceutical composition including the engineered cell of any one of the compositions provided for herein and a pharmaceutically acceptable carrier, a pharmaceutically acceptable excipient, or combination thereof.

In some embodiments, the extracellular ligand binding domain binds to a ligand selected from: a protein complex, a protein, a peptide, a receptor-binding domain, a nucleic acid, a small molecule, and a chemical agent.

In some embodiments, the extracellular ligand binding domain includes an antibody, or antigen-binding fragment thereof. In some embodiments, the extracellular ligand binding domain includes a F(ab) fragment, a F(ab′) fragment, a single chain variable fragment (scFv), or a single-domain antibody (sdAb).

In some of these embodiments, the ligand is a tumor-associated antigen. In some of these embodiments, the ligand is not expressed on a tumor cell. In some of these embodiments, the ligand is expressed on a non-tumor cell. In some of these embodiments, the ligand is expressed on cells of a healthy tissue.

In some embodiments, the extracellular ligand binding domain includes a dimerization domain. In some embodiments, the ligand further includes a cognate dimerization domain.

In some embodiments, the ligand is a cell surface ligand. In some embodiments, the cell surface ligand is expressed on a cell that further expresses a cognate ligand of the immune receptor.

In some embodiments, the membrane localization domain of a chimeric receptor of the present disclosure further includes at least a portion of an extracellular domain. In some embodiments, the membrane localization domain further includes at least a portion of an intracellular domain. In some embodiments, the membrane localization domain further includes at least a portion of an extracellular domain and at least a portion of an intracellular domain.

In some embodiments, the membrane localization domain includes a transmembrane domain selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CD8 transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAGS transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain. In some embodiments, the membrane localization domain further includes at least a portion of a corresponding extracellular domain and/or at least a portion of a corresponding intracellular domain. In some embodiments, the LAT mutant is a LAT(CA) mutant.

In some embodiments, the membrane localization domain directs and/or segregates the chimeric inhibitory receptor to a domain of a cell membrane. In some embodiments, the membrane localization domain localizes the chimeric inhibitory receptor to a lipid raft or a heavy lipid raft. In some embodiments, the membrane localization domain interacts with one or more cell membrane components localized in a domain of a cell membrane. In some embodiments, the membrane localization domain is sufficient to mitigate constitutive inhibition of immune receptor activation by the enzymatic inhibitory domain in the absence of the extracellular ligand binding domain binding a cognate ligand.

In some embodiments, the membrane localization domain mediates localization of the chimeric inhibitory receptor to a domain of a cell membrane that is distinct from domains of the cell membrane occupied by one or more components of an immune receptor in the absence of the extracellular ligand binding domain binding a cognate ligand.

In some embodiments, the membrane localization domain further includes proximal protein fragments. In some embodiments, the membrane localization domain further includes one or more intracellular inhibitory co-signaling domains. In some embodiments, the one or more intracellular inhibitory co-signaling domains of a chimeric protein include one or more ITIM-containing proteins, or fragments thereof. In some embodiments, the one or more ITIM-containing proteins, or fragments thereof, are selected from PD-1, CTLA4, TIGIT, BTLA, and LAIR1. In some embodiments, the one or more intracellular inhibitory co-signaling domains include one or more non-ITIM scaffold proteins, or fragments thereof. In some embodiments, the one or more non-ITIM scaffold proteins, or fragments thereof, are selected from GRB-2, Dok-1, Dok-2, SLAP1, SLAP2, LAGS, HAVR, GITR, and PD-L1.

In some embodiments, the extracellular ligand binding domain of a chimeric inhibitory receptor of the present disclosure is linked to the membrane localization domain through an extracellular linker region.

In some embodiments, the extracellular linker region is positioned between the extracellular ligand binding domain and membrane localization domain and operably and/or physically linked to each of the extracellular ligand binding domain and the membrane localization domain. In some embodiments, the extracellular linker region is derived from a protein selected from the group consisting of: CD8alpha, CD4, CD7, CD28, IgG1, IgG4, FcgammaRIIIalpha, LNGFR, and PDGFR. In some embodiments, the extracellular linker region comprises an amino acid sequence selected from the group consisting of: AAAIEVMYPPPYLDNEKSNGTIIHVKGKHLCPSPLFPGPSKP (SEQ ID NO:46), ESKYGPPCPSCP (SEQ ID NO:47), ESKYGPPAPSAP (SEQ ID NO:48), ESKYGPPCPPCP (SEQ ID NO:49), EPKSCDKTHTCP (SEQ ID NO:50), AAAFVPVFLPAKPTTTPAPRPPTPAPTIAS QPLSLRPEACRPAAGGAVHTRGLDFACDI YIWAPLAGTCGVLLLSLVITLYCNHRN (SEQ ID NO:51), TTTPAPRPPTPAPTIALQPLSLRPEACRPAAGGAVHTRGLDFACD (SEQ ID NO:52), ACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQ NTVCEECPDGTYSDEADAEC (SEQ ID NO:53), ACPTGLYTHSGECCKACNLGEGVAQPCGANQTVC (SEQ ID NO:54), and AVGQDTQEVIVVPHSLPFKV (SEQ ID NO:55). In some embodiments, the extracellular linker region comprises an amino acid sequence selected from the group consisting of: GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), GSTSGSGKPGSGEGSTKG (SEQ ID NO: 44), and EAAAKEAAAKEAAAKEAAAK (SEQ ID NO: 45).

In some embodiments, the chimeric inhibitory receptor further comprises an intracellular spacer region positioned between the membrane localization domain and the enzymatic inhibitory domain and operably and/or physically linked to each of the membrane localization domain and the enzymatic inhibitory domain. In some embodiments, the intracellular spacer region comprises an amino acid sequence selected from the group consisting of: GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), GSTSGSGKPGSGEGSTKG (SEQ ID NO: 44), and EAAAKEAAAKEAAAKEAAAK (SEQ ID NO: 45). In some embodiments, the intracellular spacer region comprises an amino acid sequence selected from the group consisting of:

(SEQ ID NO: 46)
AAAIEVMYPPPYLDNEKSNGTIIHVKGKHLCPSPLFPGPSKP,
(SEQ ID NO: 47)
ESKYGPPCPSCP,
(SEQ ID NO: 48)
ESKYGPPAPSAP,
(SEQ ID NO: 49)
ESKYGPPCPPCP,
(SEQ ID NO: 50)
EPKSCDKTHTCP,
(SEQ ID NO: 51)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 52)
TTTPAPRPPTPAPTIALQPLSLRPEACRPAAGGAVHTRGLDFACD.
(SEQ ID NO: 53)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 54)
ACPTGLYTHSGECCKACNLGEGVAQPCGANQTVC,
and
(SEQ ID NO: 55)
AVGQDTQEVIVVPHSLPFKV.

In some embodiments, the enzymatic inhibitory domain of a chimeric inhibitory receptor of the present disclosure includes at least a portion of an extracellular domain, a transmembrane domain, and/or an intracellular domain. In some embodiments, the enzymatic inhibitory domain includes an enzyme catalytic domain.

In some embodiments, the enzymatic inhibitory domain includes at least a portion of an enzyme. In some embodiments, the portion of the enzyme includes an enzyme domain or an enzyme fragment. In some embodiments, the portion of the enzyme is a catalytic domain of the enzyme.

In some embodiments, the enzyme is selected from the group consisting of: CSK, SHP-1, SHP-2, PTEN, CD45, CD148, PTP-MEG1, PTP-PEST, c-CBL, CBL-b, PTPN22, LAR, PTPH1, SHIP-1, ZAP70, and RasGAP.

In some embodiments, the enzymatic inhibitory domain is derived from CSK. In some embodiments, the enzymatic inhibitory domain comprises a CSK protein with a SRC homology 3 (SH3) deletion.

In some embodiments, the enzymatic inhibitory domain is derived from SHP-1. In some embodiments, the enzymatic inhibitory domain comprises a protein tyrosine phosphatase (PTP) domain.

In some embodiments, the enzymatic inhibitory domain is derived from SHP-2.

In some embodiments, the enzymatic inhibitory domain is derived from PTEN.

In some embodiments, the enzymatic inhibitory domain is derived from CD45.

In some embodiments, the enzymatic inhibitory domain is derived from CD148.

In some embodiments, the enzymatic inhibitory domain is derived from PTP-MEG1.

In some embodiments, the enzymatic inhibitory domain is derived from PTP-PEST.

In some embodiments, the enzymatic inhibitory domain is derived from c-CBL.

In some embodiments, the enzymatic inhibitory domain is derived from CBL-b.

In some embodiments, the enzymatic inhibitory domain is derived from PTPN22.

In some embodiments, the enzymatic inhibitory domain is derived from LAR.

In some embodiments, the enzymatic inhibitory domain is derived from PTPH1.

In some embodiments, the enzymatic inhibitory domain is derived from SHIP-1. In some embodiments, the enzymatic inhibitory domain comprises a protein tyrosine phosphatase (PTP) domain.

In some embodiments, the enzymatic inhibitory domain is derived from ZAP70. In some embodiments, the enzymatic inhibitory domain comprises a SRC homology 1 (SH1) domain, a SRC homology 2 (SH2) domain, or an SH1 domain and an SH2 domain. In some embodiments, the enzymatic inhibitory domain comprises a ZAP70 protein with a kinase domain deletion. In some embodiments, wherein the enzymatic inhibitory domain comprises a mutant ZAP70 protein with a Tyr492Phe amino acid substitution, a Tyr493Phe amino acid substitution, or a Tyr492Phe amino acid substitution and a Tyr493Phe amino acid substitution.

In some embodiments, the enzymatic inhibitory domain is derived from RasGAP.

In some embodiments, the enzymatic inhibitory domain includes one or more modifications that modulate basal inhibition. In some embodiments, the one or more modifications reduce basal inhibition. In other embodiments, the one or more modifications increase basal inhibition.

In some embodiments, the enzymatic inhibitory domain inhibits immune receptor activation upon recruitment of the chimeric inhibitory receptor proximal to an immune receptor.

In some embodiments, the immune receptor is a chimeric immune receptor. In some embodiments, the immune receptor is a chimeric antigen receptor. In some embodiments, the immune receptor is a naturally-occurring immune receptor. In some embodiments, the immune receptor is a naturally-occurring antigen receptor.

In some embodiments, the immune receptor is selected from a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor.

In some embodiments, the immune receptor is a T cell receptor.

In some embodiments, a genetically engineered cell of the present disclosure further includes at least one immune receptor. In some embodiments, the at least one immune receptor is a chimeric immune receptor. In some embodiments, the at least one immune receptor is a chimeric antigen receptor. In some embodiments, the at least one immune receptor is a naturally-occurring immune receptor. In some embodiments, the at least one immune receptor is a naturally-occurring antigen receptor. In some embodiments, the at least one immune receptor is selected from a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor.

In some embodiments, a chimeric inhibitory receptor of the present disclosure inhibits immune receptor activation upon ligand binding when proximal to the immune receptor.

In some embodiments, the ligand is a cell surface ligand. In some embodiments, the cell surface ligand is expressed on a cell that further expresses a cognate immune receptor ligand. In some embodiments, ligand binding to the chimeric inhibitory receptor and cognate immune receptor ligand binding to the immune receptor localizes the chimeric inhibitory receptor proximal to the immune receptor. In some embodiments, localization of the chimeric inhibitory receptor proximal to the immune receptor inhibits immune receptor activation.

In some embodiments, the cell is a T cell. In some embodiments, the immune receptor is a T cell receptor. In some embodiments, immune receptor activation is T cell activation.

In some embodiments, a genetically engineered cell of the present disclosure is an immunomodulatory cell. In some embodiments, the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

In some embodiments, the cell is autologous. In some embodiments, the cell is allogeneic.

Also provided herein are methods of inhibiting immune receptor activation. The methods include: contacting a genetically engineered cell or a pharmaceutical composition disclosed herein under conditions suitable for the chimeric inhibitory receptor to bind the cognate ligand, wherein, when localized proximal to an immune receptor expressed on a cell membrane of the engineered cell, the chimeric inhibitory inhibits immune receptor activation.

Also provided herein are methods for reducing an immune response. The methods include: administering a genetically engineered cell or a pharmaceutical composition disclosed herein to a subject in need of such treatment.

Also provided herein are methods for preventing, attenuating, or inhibiting a cell-mediated immune response induced by a tumor-targeting chimeric receptor expressed on the surface of an immunomodulatory cell. The methods include: administering a genetically engineered cell or a pharmaceutical composition disclosed herein to a subject in need of such treatment.

Also provided herein are methods for preventing, attenuating, or inhibiting a cell-mediated immune response induced by a tumor-targeting chimeric receptor expressed on the surface of an immunomodulatory cell. The methods include: contacting a genetically engineered cell or a pharmaceutical composition disclosed herein with a cognate ligand of the chimeric inhibitory receptor under conditions suitable for the chimeric inhibitory receptor to bind the cognate ligand, wherein, upon binding of the ligand to the chimeric inhibitory receptor, the enzymatic inhibitory domain prevents, attenuates, or inhibits activation of the tumor-targeting chimeric receptor.

Also provided herein are methods of treating an autoimmune disease or disease treatable by reducing an immune response. The methods include: administering a genetically engineered cell or a pharmaceutical composition disclosed herein to a subject in need of such treatment.

These and other aspects are descried in more detail below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1. Schematic depicting a mechanism whereby a chimeric inhibitory receptor of the present disclosure blocks T cell activation.

FIG. 2. Schematic depicting a composition of certain embodiments of a chimeric inhibitory receptor. ELBD: Extracellular Ligand Binding Domain—examples include, but are not limited to, scFv (e.g., against tumor antigen), natural receptor/ligand domains, and orthogonal dimerization domains (e.g., leucine zippers that could engage with a soluble targeting molecule); MLD: Membrane Localization Domain (optionally including proximal intra- and extra-cellular segments involved in localization to sub-domains of the cell membrane (e.g., lipid rafts)—examples include, but are not limited to, the transmembrane domains of LAX, CD25, CD7 (Pavel Otáhal et al., Biochim Biophys Acta. 2011 February; 1813(2):367-76) and mutants of LAT (e.g. LAT(CA); see e.g., Kosugi A., et al. Involvement of SHP-1 tyrosine phosphatase in TCR-mediated signaling pathways in lipid rafts, Immunity, 2001 June; 14(6): 669-80, the entirety of which is incorporated herein); EID: Enzymatic Inhibitory Domain (e.g., enzymes that inhibit the native T cell activation cascade, including domains, fragments, or mutants of enzymes, selected to maximize efficacy and minimize basal inhibition)—examples include, but are not limited to, CSK (Pavel Otáhal et al., Biochim Biophys Acta. 2011 February; 1813(2):367-76), SHP-1 (see e.g., Kosugi A., et al. Involvement of SHP-1 tyrosine phosphatase in TCR-mediated signaling pathways in lipid rafts, Immunity, 2001 June; 14(6): 669-80), PTEN, CD45, CD148, PTP-MEG1, PTP-PEST, c-CBL, CBL-b, LYP/Pep/PTPN22, LAR, PTPH1, SHIP-1, RasGAP (see e.g., Stanford et al., Regulation of TCR signaling by tyrosine phosphatases: from immune homeostasis to autoimmunity, Immunology, 2012 September; 137(1): 1-19, the entirety of which is incorporated herein).

FIG. 3. Schematic depicting a composition of certain embodiments of a chimeric inhibitory receptor (e.g., an “extended” chimeric inhibitory receptor). ELBD, MLD, and EID as described for FIG. 2. SID: Scaffold Inhibitory Domain—examples include, but are not limited to, ITIM containing protein domains (e.g. cytoplasmic tails of PD-1, CTLA4, TIGIT, BTLA, and/or LAIR1), or fragment(s) thereof) and non-ITIM scaffold protein domains, or fragment(s) thereof, that inhibit T cell activation, including GRB-2, Dok-1, Dok-2, SLAP, LAGS, HAVR, GITR, and PD-L1.

FIG. 4. Schematic illustrating a NOT-gate aCAR/iCAR system. A T cell was engineered to express an anti-CD19 iCAR, including a CSK domain as the EID domain, to inhibit signaling of a co-expressed aCAR that included a CD28-CD3 intracellular signaling domain. Target k562 cells were engineered to express a cognate antigen for an aCAR (CD20) or engineered to express both the cognate antigen for the aCAR (CD20) and a cognate antigen for an iCAR (CD19).

FIG. 5. Representative flow-cytometry plots demonstrating expression of iCAR construct anti-CD19_scFv-Csk fusions at levels detectable above unmodified cells following transduction of CD4+ and CD8+ T cells without subsequent enrichment.

FIG. 6. Expression profiles as assessed by flow-cytometry for aCAR and iCAR constructs. Shown is: aCAR+=cells that express the aCAR (w/ and w/out iCAR) [first column]; iCAR+=cells that express the iCAR (w/ and w/out the aCAR) [second column]; and dual+=cells that express both the aCAR and iCAR [third column].

FIG. 7. Efficacy of iCAR inhibition of aCAR signaling as assessed by killing efficiency, represented as ratio of killing CD19/CD20 targets cells to CD20-only target cells. Shown is: transduction with an aCAR construct only (left column); co-transduction of T cells with an iCAR possessing a CSK enzymatic inhibitory domain (iCAR31) and an aCAR (middle column); and co-transduction of T cells with an iCAR possessing a CSK enzymatic inhibitory domain including an SH3 deletion (iCAR26) and an aCAR (right column).

### DETAILED DESCRIPTION

### Definitions

Terms used in the claims and specification are defined as set forth below unless otherwise specified.

The term “chimeric inhibitory receptor” or “inhibitory chimeric antigen receptor” or “inhibitory chimeric receptor” as used herein refers to a polypeptide or a set of polypeptides, which when expressed in a cell, such as an immune effector cell, provides the cell with specificity for a target cell and the ability to negatively regulate intracellular signal transduction. An chimeric inhibitory receptor may also be called an “iCAR.”

The term “tumor-targeting chimeric receptor” or “activating chimeric receptor” refers to activating chimeric receptors, tumor-targeting chimeric antigen receptors (CARs), or engineered T cell receptors having architectures capable of inducing signal transduction or changes in protein expression in the activating chimeric receptor-expressing cell that results in the initiation of an immune response. A tumor targeting chimeric receptor may also be called an “aCAR.”

The term, “transmembrane domain” as used herein, refers to a domain that spans a cellular membrane. In some embodiments, a transmembrane domain comprises a hydrophobic alpha helix.

The term “tumor” refers to tumor cells and the associated tumor microenvironment (TME). In some embodiments, tumor refers to a tumor cell or tumor mass. In some embodiments, tumor refers to the tumor microenvironment.

The term “not expressed” refers to expression that is at least 2-fold lower than the level of expression in non-tumor cells that would result in activation of the tumor-targeting chimeric antigen receptor. In some embodiments, the expression is at least 2-fold, at least 3-fold, at least 4-fold, at least 5-fold, at least 6-fold, at least 7-fold, at least 8-fold, at least 9-fold, or at least 10-fold or more lower than the level of expression in non-tumor cells that would result in activation of the tumor-targeting chimeric antigen receptor.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Chimeric Inhibitory Receptors

Provided herein are chimeric inhibitory receptors that are useful, inter alia, as a NOT logic gate for controlling immune cell activity. The chimeric inhibitory receptors include an extracellular ligand binding domain, a membrane localization domain including a transmembrane domain; and an enzymatic inhibitory domain. In some embodiments, the enzymatic inhibitory domain inhibits immune receptor activation upon recruitment of a chimeric inhibitory receptor of the present disclosure to be proximal to an immune receptor. Without wishing to be bound by theory, binding between the chimeric inhibitory receptor and its cognate ligand generally mediates spatial recruitment of the enzymatic inhibitory domain to be proximal to the immune receptor and/or downstream signaling complexes such that the enzymatic inhibitory domain is capable of negatively regulating an intracellular signal transduction cascade. Proximal can include two molecules (e.g., proteins or protein domains) physically interacting. Proximal can include two molecules being sufficiently physically close to operably interact with one another. Proximal can include two molecules physically or operably interacting with a shared intermediary molecule, e.g., a scaffold protein. Proximal can include two molecules physically or operably interacting with a shared complex, e.g., a signaling cascade. Proximal can include two molecules physically interacting for a duration of time to operably interact with one another. Proximal can include two molecules being sufficiently physically close for a duration of time to operably interact with one another. Proximal can include two molecules physically or operably interacting for a duration of time with a shared intermediary molecule, e.g., a scaffold protein. Proximal can include two molecules physically or operably interacting for a duration of time with a shared complex, e.g., a signaling cascade. Durations of time mediating operable interactions generally refers to interactions longer than stochastic interactions and can include sustained physical proximity, for example sustained ligand-mediated localization to a distinct domain of a cell membrane (e.g., an immunological synapse). Proximal to an immune receptor can include localization to a cellular environment allowing direct inhibition of the signaling activity of the immune receptor. Proximal to an immune receptor can include localization to a cellular environment allowing inhibition of an intracellular signal transduction cascade mediated by the immune receptor. The disclosed chimeric inhibitory receptors thus can be engineered to contain appropriate extracellular ligand binding domains that will reduce intracellular signaling, such as immune responses, in the presence of the cognate ligand. In some embodiments, the ligand is located on a cell surface. In some embodiments, the ligand is an agent that is not on a cell surface, such as a small molecule, secreted factor, environmental signal or other soluble and/or secreted agent that mediates spatial recruitment of the enzymatic inhibitory domain to be proximal to the immune receptor, such as a cross-linking reagent, a small molecule that mediates heterodimerization of protein domains, or antibody, each that can mediate spatial recruitment of the enzymatic inhibitory domain to be proximal to the immune receptor. Uses of the chimeric inhibitory receptors include, but are not limited to, reducing immune responses, controlling T cell activation, controlling CAR-T responses, and treating autoimmune diseases or any disease that is treatable by reducing immune responses.

Provided herein, in some aspects, are chimeric inhibitory receptors comprising: an extracellular ligand binding domain; a membrane localization domain, wherein the membrane localization domain comprises a transmembrane domain; and an enzymatic inhibitory domain, wherein the enzymatic inhibitory domain inhibits immune receptor activation when proximal to an immune receptor.

### Enzymatic Inhibitory Domains

As used herein, the term “enzymatic inhibitory domain” refers to a protein domain having an enzymatic function that inhibits an intracellular signal transduction cascade, for example a native T cell activation cascade. For example, enzymatic inhibitory domains can be an enzyme, or catalytic domain of an enzyme, whose enzymatic activity mediates negative regulation of intracellular signal transduction. Non-limiting examples of enzymes and enzymatic functions capable of negatively regulating intracellular signal transduction include (1) a kinase or kinase domain whose enzymatic phosphorylation activity mediates negative regulation of intracellular signal transduction, (2) a phosphatase or phosphatase domain whose enzymatic phosphatase activity mediates negative regulation of intracellular signal transduction, and/or (3) a ubiquitin ligase whose enzymatic ubiquitination activity mediates negative regulation of intracellular signal transduction. Enzymatic regulation of signaling (e.g., inhibition intracellular signal transduction cascades) is described in more detail in Pavel Otáhal et al. (Biochim Biophys Acta. 2011 February; 1813(2):367-76), Kosugi A., et al. (Involvement of SHP-1 tyrosine phosphatase in TCR-mediated signaling pathways in lipid rafts, Immunity, 2001 June; 14(6): 669-80), and Stanford, et al. (Regulation of TCR signaling by tyrosine phosphatases: from immune homeostasis to autoimmunity, Immunology, 2012 September; 137(1): 1-19), each of which is incorporated herein by reference for all purposes.

In some embodiments, the enzymatic inhibitory domain of a chimeric inhibitory receptor of the present disclosure comprises at least a portion of an extracellular domain, a transmembrane domain, and/or an intracellular domain. In some embodiments, the enzymatic inhibitory domain comprises at least a portion of an enzyme, such as a biologically active portion of an enzyme. In some embodiments, the portion of the enzyme comprises an enzyme domain(s), an enzyme fragment(s), or a mutant(s) thereof, such as a kinase domain or a phosphatase domain and mutant thereof. In some embodiments, the portion of the enzyme is a catalytic domain of the enzyme, such as the portion of an enzyme having kinase or phosphatase catalytic activity. In some embodiments, the enzyme domain(s), enzyme fragment(s), or mutants(s) thereof are selected to maximize efficacy and minimize basal inhibition.

In some embodiments, the enzymatic inhibitory domain comprises one or more modifications that modulate basal inhibition. Examples of modifications include, but are not limited to, truncation mutation(s), amino acid substitution(s), introduction of locations for post-translational modification (examples of which are known to those having skill in the art), and addition of new functional groups. In some embodiments, the enzyme domain(s), enzyme fragment(s), or mutants(s) thereof are selected to maximize efficacy and minimize basal inhibition. In some embodiments, the one or more modifications reduce basal inhibition. In other embodiments, the one or more modifications increase basal inhibition. In a non-limiting illustrative example and without wishing to be bound by theory, deletion of an SH3 domain (e.g., in a CSK enzyme) can minimize constitutive clustering/signaling (i.e., in the absence of ligand binding) and thereby lower the basal level enzymatic inhibitory activity of a chimeric inhibitory receptor.

In some embodiments, ligand binding between the chimeric inhibitory receptor and its cognate ligand can mediate localization of the chimeric inhibitory receptor to a cellular environment where the enzymatic inhibitory domain is proximal to an intracellular signaling domain or an immune receptor allowing direct inhibition of the signaling activity of the immune receptor. In a non-limiting illustrative example, binding between the chimeric inhibitory receptor expressed on a T cell and its cognate ligand can cause localization of the enzymatic inhibitory domain to be proximal to a TCR or CAR intracellular signaling domain (e.g., localized to a immunological synapse) such that the enzymatic inhibitory domain is capable of negatively regulating T cell signaling and/or activation. In some embodiments, ligand binding between the chimeric inhibitory receptor and its cognate ligand can mediate localization of the chimeric inhibitory receptor to a cellular environment where the enzymatic inhibitory domain is proximal to an immune receptor allowing inhibition of an intracellular signal transduction cascade mediated by the immune receptor. In some embodiments, ligand binding between the chimeric inhibitory receptor and its cognate ligand can mediate spatial clustering of multiple chimeric inhibitory receptors proximal to an immune receptor such that the clustering of the enzymatic inhibitory domains facilitates their inhibitory activity on the immune receptor.

In some embodiments, the enzyme is selected from CSK, SHP-1, SHP-2, PTEN, CD45, CD148, PTP-MEG1, PTP-PEST, c-CBL, CBL-b, PTPN22, LAR, PTPH1, SHIP-1, ZAP70, and RasGAP.

In some embodiments, the enzymatic inhibitory domain has a SRC homology 3 (SH3) domain. In some embodiments, the enzymatic inhibitory domain is derived from a protein with a SRC homology 3 (SH3) deletion. In some embodiments, the enzymatic inhibitory domain has a protein tyrosine phosphatase (PTP) domain. In some embodiments, the enzymatic inhibitory domain includes a SRC homology 1 (SH1) domain, a SRC homology 2 (SH2) domain, or an SH1 domain and an SH2 domain.

In some embodiments, the enzymatic inhibitory domain is derived from a protein with a kinase domain deletion or mutation(s) reducing kinase activity. In some embodiments, the enzymatic inhibitory domain is derived from a protein with a kinase domain deletion or mutation(s) reducing kinase activity generating a dominant negative kinase mutant. In a non-limiting illustrative example and without wishing to be bound by theory, a chimeric inhibitory receptor including enzymatic inhibitory domain having a deletion or mutation of a kinase domain (e.g., in a ZAP70 enzyme) can act as a dominant negative kinase-dead protein and reduce or eliminate an intracellular signaling cascade through competition with the corresponding native wild-type protein that was the source of the enzymatic inhibitory domain.

In some embodiments, the enzymatic inhibitory domain is derived from CSK. In some embodiments, the enzymatic inhibitory domain derived from CSK is a CSK protein with a SRC homology 3 (SH3) deletion.

In some embodiments, the enzymatic inhibitory domain is derived from SHP-1. In some embodiments, the enzymatic inhibitory domain derived from SHP-1 has a tyrosine phosphatase (PTP) domain.

In some embodiments, the enzymatic inhibitory domain is derived from SHP-2. In some embodiments, the enzymatic inhibitory domain is derived from PTEN. In some embodiments, the enzymatic inhibitory domain is derived from CD45. In some embodiments, the enzymatic inhibitory domain is derived from CD148. In some embodiments, the enzymatic inhibitory domain is derived from PTP-MEG1. In some embodiments, the enzymatic inhibitory domain is derived from PTP-PEST. In some embodiments, the enzymatic inhibitory domain is derived from c-CBL. In some embodiments, the enzymatic inhibitory domain is derived from CBL-b. In some embodiments, the enzymatic inhibitory domain is derived from PTPN22. In some embodiments, the enzymatic inhibitory domain is derived from LAR. In some embodiments, the enzymatic inhibitory domain is derived from PTPH1.

In some embodiments, the enzymatic inhibitory domain is derived from SHIP-1. In some embodiments, the enzymatic inhibitory domain is derived from SHIP-1 has a protein tyrosine phosphatase (PTP) domain.

In some embodiments, the enzymatic inhibitory domain is derived from ZAP70. In some embodiments, the enzymatic inhibitory domain derived from ZAP70 has a SRC homology 1 (SH1) domain, a SRC homology 2 (SH2) domain, or an SH1 domain and an SH2 domain. In some embodiments, the enzymatic inhibitory domain derived from ZAP70 has a kinase domain deletion. In some embodiments, the enzymatic inhibitory domain derived from ZAP70 has a Tyr492Phe amino acid substitution, a Tyr493Phe amino acid substitution, or a Tyr492Phe amino acid substitution and a Tyr493Phe amino acid substitution.

In some embodiments, the enzymatic inhibitory domain is derived from RasGAP.

Exemplary sequences for enzymatic inhibitory domains are shown in Table 1A and Table 1B. In some embodiments, an enzymatic inhibitory domain is any of the amino acid sequences listed in Table 1A. In some embodiments, an enzymatic inhibitory domain has an amino acid sequence that is at least 90%, at least 91%, at least 92%, at least 93%, at least 94%, at least 95%, at least 96%, at least 97%, at least 98%, or at least 99% identical to any of the amino acid sequences listed in Table 1A. In some embodiments, an enzymatic inhibitory domain is encoded by a nucleic acid sequence that is at least 60%, at least 65%, at least 70%, at least 75%, at least 80%, or at least 85% identical to any of the nucleic acid sequences listed in Table 1B. In some embodiments, an enzymatic inhibitory domain is encoded by a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 1B.

TABLE 1A
Enzymatic Inhibitory Domains Amino Acid Sequences

| Domain | Amino Acid Sequence |
|---|---|
| Csk | SAIQAAWPSGTECIAKYNFHGTAEQDLPFCKGDVLTIVAVTKDPNWYKAKNKVGR |
| | EGIIPANYVQKREGVKAGTKLSLMPWFHGKITREQAERLLYPPETGLFLVRESTNYP |
| | GDYTLCVSCDGKVEHYRIMYHASKLSIDEEVYFENLMQLVEHYTSDADGLCTRLIK |
| | PKVMEGTVAAQDEFYRSGWALNMKELKLLQTIGKGEFGDVMLGDYRGNKVAVK |
| | CIKNDATAQAFLAEASVMTQLRHSNLVQLLGVIVEEKGGLYIVTEYMAKGSLVDYL |
| | RSRGRSVLGGDCLLKFSLDVCEAMEYLEGNNFVHRDLAARNVLVSEDNVAKVSDF |
| | GLTKEASSTQDTGKLPVKWTAPEALREKKFSTKSDVWSFGILLWEIYSFGRVPYPRI |
| | PLKDVVPRVEKGYKMDAPDGCPPAVYEVMKNCWHLDAAMRPSFLQLREQLEHIK |
| | THELHL (SEQ ID NO: 1) |
| Csk deltaSH3 | VKAGTKLSLMPWFHGKITREQAERLLYPPETGLFLVRESTNYPGDYTLCVSCDGKV |
| | EHYRIMYHASKLSIDEEVYFENLMQLVEHYTSDADGLCTRLIKPKVMEGTVAAQDE |
| | FYRSGWALNMKELKLLQTIGKGEFGDVMLGDYRGNKVAVKCIKNDATAQAFLAE |
| | ASVMTQLRHSNLVQLLGVIVEEKGGLYIVTEYMAKGSLVDYLRSRGRSVLGGDCL |
| | LKFSLDVCEAMEYLEGNNFVHRDLAARNVLVSEDNVAKVSDFGLTKEASSTQDTG |
| | KLPVKWTAPEALREKKFSTKSDVWSFGILLWEIYSFGRVPYPRIPLKDVVPRVEKGY |
| | KMDAPDGCPPAVYEVMKNCWHLDAAMRPSFLQLREQLEHIKTHELHL (SEQ ID |
| | NO: 2) |
| SHP-1 | MVRWFHRDLSGLDAETLLKGRGVHGSFLARPSRKNQGDFSLSVRVGDQVTHIRIQN |
| | SGDFYDLYGGEKFATLTELVEYYTQQQGVLQDRDGTIIHLKYPLNCSDPTSERWYH |
| | GHMSGGQAETLLQAKGEPWTFLVRESLSQPGDFVLSVLSDQPKAGPGSPLRVTHIK |
| | VMCEGGRYTVGGLETFDSLTDLVEHFKKTGIEEASGAFVYLRQPYYATRVNAADIE |
| | NRVLELNKKQESEDTAKAGFWEEFESLQKQEVKNLHQRLEGQRPENKGKNRYKNI |
| | LPFDHSRVILQGRDSNIPGSDYINANYIKNQLLGPDENAKTYIASQGCLEATVNDFW |
| | QMAWQENSRVIVMTTREVEKGRNKCVPYWPEVGMQRAYGPYSVTNCGEHDTTEY |
| | KLRTLQVSPLDNGDLIREIWHYQYLSWPDHGVPSEPGGVLSFLDQINQRQESLPHAG |
| | PIIVHCSAGIGRTGTIIVIDMLMENISTKGLDCDIDIQKTIQMVRAQRSGMVQTEAQY |
| | KFIYVAIAQFIETTKKKLEVLQSQKGQESEYGNITYPPAMKNAHAKASRTSSKHKED |
| | VYENLHTKNKREEKVKKQRSADKEKSKGSLKRK (SEQ ID NO: 3) |
| SHP-1PTP | FWEEFESLQKQEVKNLHQRLEGQRPENKGKNRYKNILPFDHSRVILQGRDSNIPGSD |
| domain | YINANYIKNQLLGPDENAKTYIASQGCLEATVNDFWQMAWQENSRVIVMTTREVE |
| | KGRNKCVPYWPEVGMQRAYGPYSVTNCGEHDTTEYKLRTLQVSPLDNGDLIREIW |
| | HYQYLSWPDHGVPSEPGGVLSFLDQINQRQESLPHAGPIIVHCSAGIGRTGTIIVIDM |
| | LMENISTKGLDCDIDIQKTIQMVRAQRSGMVQTEAQYKFIYVAIAQF (SEQ ID |
| | NO: 4) |
| SHP-2 | MTSRRWFHPNITGVEAENLLLTRGVDGSFLARPSKSNPGDFTLSVRRNGAVTHIKIQ |
| | NTGDYYDLYGGEKFATLAELVQYYMEHHGQLKEKNGDVIELKYPLNCADPTSER |
| | WFHGHLSGKEAEKLLTEKGKHGSFLVRESQSHPGDFVLSVRTGDDKGESNDGKSK |
| | VTHVMIRCQELKYDVGGGERFDSLTDLVEHYKKNPMVETLGTVLQLKQPLNTTRI |
| | NAAEIESRVRELSKLAETTDKVKQGFWEEFETLQQQECKLLYSRKEGQRQENKNKN |
| | RYKNILPFDHTRVVLHDGDPNEPVSDYINANIIMPEFETKCNNSKPKKSYIATQGCL |
| | QNTVNDFWRMVFQENSRVIVMTTKEVERGKSKCVKYWPDEYALKEYGVMRVRN |
| | VKESAAHDYTLRELKLSKVGQALLQGNTERTVWQYHFRTWPDHGVPSDPGGVLD |
| | FLEEVHHKQESIMDAGPVVVHCSAGIGRTGTFIVIDILIDIIREKGVDCDIDVPKTIQM |
| | VRSQRSGMVQTEAQYRFIYMAVQHYIETLQRRIEEEQKSKRKGHEYTNIKYSLADQ |
| | TSGDQSPLPPCTPTPPCAEMREDSARVYENVGLMQQQKSFR (SEQ ID NO: 5) |
| CD45 | YKIYDLHKKRSCNLDEQQELVERDDEKQLMNVEPIHADILLETYKRKIADEGRLFL |
| | AEFQSIPRVFSKFPIKEARKPFNQNKNRYVDILPYDYNRVELSEINGDAGSNYINASY |
| | IDGFKEPRKYIAAQGPRDETVDDFWRMIWEQKATVIVMVTRCEEGNRNKCAEYWP |
| | SMEEGTRAFGDVVVKINQHKRCPDYIIQKLNIVNKKEKATGREVTHIQFTSWPDHG |
| | VPEDPHLLLKLRRRVNAFSNFFSGPIVVHCSAGVGRTGTYIGIDAMLEGLEAENKVD |
| | VYGYVVKLRRQRCLMVQVEAQYILIHQALVEYNQFGETEVNLSELHPYLHNMKKR |
| | DPPSEPSPLEAEFQRLPSYRSWRTQHIGNQEENKSKNRNSNVIPYDYNRVPLKHELE |
| | MSKESEHDSDESSDDDSDSEEPSKYINASFIMSYWKPEVMIAAQGPLKETIGDFWQ |
| | MIFQRKVKVIVMLTELKHGDQEICAQYWGEGKQTYGDIEVDLKDTDKSSTYTLRVF |
| | ELRHSKRKDSRTVYQYQYTNWSVEQLPAEPKELISMIQVVKQKLPQKNSSEGNKHH |
| | KSTPLLIHCRDGSQQTGIFCALLNLLESAETEEVVDIFQVVKALRKARPGMVSTFEQ |
| | YQFLYDVIASTYPAQNGQVKKNNHQEDKIEFDNEVDKVKQDANCVNPLGAPEKLP |
| | EAKEQAEGSEPTSGTEGPEHSVNGPASPALNQGS (SEQ ID NO: 6) |
| Grb2 | MEAIAKYDFKATADDELSFKRGDILKVLNEECDQNWYKAELNGKDGFIPKNYIEM |
| | KPHPWFFGKIPRAKAEEMLSKQRHDGAFLIRESESAPGDFSLSVKFGNDVQHFKVL |
| | RDGAGKYFLWVVKFNSLNELVDYHRSTSVSRNQQIFLRDIEQVPQQPTYVQALFDF |
| | DPQEDGELGFRRGDFIHVMDNSDPNWWKGACHGQTGMFPRNYVTPVNRNV (SEQ |
| | ID NO: 7) |
| PTEN | MTAIIKEIVSRNKRRYQEDGFDLDLTYIYPNIIAMGFPAERLEGVYRNNIDDVVRFLD |
| | SKHKNHYKIYNLCAERHYDTAKFNCRVAQYPFEDHNPPQLELIKPFCEDLDQWLSE |
| | DDNHVAAIHCKAGKGRTGVMICAYLLHRGKFLKAQEALDFYGEVRTRDKKGVTIP |
| | SQRRYVYYYSYLLKNHLDYRPVALLFHKMMFETIPMFSGGTCNPQFVVCQLKVKI |
| | YSSNSGPTRREDKFMYFEFPQPLPVCGDIKVEFFHKQNKMLKKDKMFHFWVNTFFI |
| | PGPEETSEKVENGSLCDQEIDSICSIERADNDKEYLVLTLTKNDLDKANKDKANRYF |
| | SPNFKVKLYFTKTVEEPSNPEASSSTSVTPDVSDNEPDHYRYSDTTDSDPENEPFDED |
| | QHTQITKV (SEQ ID NO: 8) |
| PTP-MEG1 | MTSRFRLPAGRTYNVRASELARDRQHTEVVCNILLLDNTVQAFKVNKHDQGQVLL |
| | DVVFKHLDLTEQDYFGLQLADDSTDNPRWLDPNKPIRKQLKRGSPYSLNFRVKFFV |
| | SDPNKLQEEYTRYQYFLQIKQDILTGRLPCPSNTAALLASFAVQSELGDYDQSENLS |
| | GYLSDYSFIPNQPQDFEKEIAKLHQQHIGLSPAEAEFNYLNTARTLELYGVEFHYAR |
| | DQSNNEIMIGVMSGGILIYKNRVRMNTFPWLKIVKISFKCKQFFIQLRKELHESRETL |
| | LGFNMVNYRACKNLWKACVEHHTFFRLDRPLPPQKNFFAHYFTLGSKFRYCGRTE |
| | VQSVQYGKEKANKDRVFARSPSKPLARKLMDWEVVSRNSISDDRLETQSLPSRSPP |
| | GTPNHRNSTFTQEGTRLRPSSVGHLVDHMVHTSPSEVFVNQRSPSSTQANSIVLESSP |
| | SQETPGDGKPPALPPKQSKKNSWNQIHYSHSQQDLESHINETFDIPSSPEKPTPNGGIP |
| | HDNLVLIRMKPDENGRFGFNVKGGYDQKMPVIVSRVAPGTPADLCVPRLNEGDQV |
| | VLINGRDIAEHTHDQVVLFIKASCERHSGELMLLVRPNAVYDVVEEKLENEPDFQYI |
| | PEKAPLDSVHQDDHSLRESMIQLAEGLITGTVLTQFDQLYRKKPGMTMSCAKLPQN |
| | ISKNRYRDISPYDATRVILKGNEDYINANYINMEIPSSSIINQYIACQGPLPHTCTDFW |
| | QMTWEQGSSMVVMLTTQVERGRVKCHQYWPEPTGSSSYGCYQVTCHSEEGNTAY |
| | IFRKMTLFNQEKNESRPLTQIQYIAWPDHGVPDDSSDFLDFVCHVRNKRAGKEEPV |
| | VVHCSAGIGRTGVLITMETAMCLIECNQPVYPLDIVRTMRDQRAMMIQTPSQYRFV |
| | CEAILKVYEEGFVKPLTTSTNK (SEQ ID NO: 9) |
| PTPN22 | DQREILQKFLDEAQSKKITKEEFANEFLKLKRQSTKYKADKTYPTTVAEKPKNIKKN |
| | RYKDILPYDYSRVELSLITSDEDSSYINANFIKGVYGPKAYIATQGPLSTTLLDFWRM |
| | IWEYSVLIIVMACMEYEMGKKKCERYWAEPGEMQLEFGPFSVSCEAEKRKSDYIIR |
| | TLKVKFNSETRTIYQFHYKNWPDHDVPSSIDPILELIWDVRCYQEDDSVPICIHCSAG |
| | CGRTGVICAIDYTWMLLKDGIIPENFSVFSLIREMRTQRPSLVQTQEQYELVYNAVL |
| | ELFKRQMDVIRDKHSGTESQAKHCIPEKNHTLQADSYSPNLPKSTTKAAKMMNQQ |
| | RTKMEIKESSSFDFRTSEISAKEELVLHPAKSSTSFDFLELNYSFDKNADTTMKWQT |
| | KAFPIVGEPLQKHQSLDLGSLLFEGCSNSKPVNAAGRYFNSKVPITRTKSTPFELIQQ |
| | RETKEVDSKENFSYLESQPHDSCFVEMQAQKVMHVSSAELNYSLPYDSKHQIRNAS |
| | NVKHHDSSALGVYSYIPLVENPYFSSWPPSGTSSKMSLDLPEKQDGTVFPSSLLPTSS |
| | TSLFSYYNSHDSLSLNSPTNISSLLNQESAVLATAPRIDDEIPPPLPVRTPESFIVVEEA |
| | GEFSPNVPKSLSSAVKVKIGTSLEWGGTSEPKKFDDSVILRPSKSVKLRSPKSELHQD |
| | RSSPPPPLPERTLESFFLADEDCMQAQSIETYSTSYPDTMENSTSSKQTLKTPGKSFTR |
| | SKSLKILRNMKKSICNSCPPNKPAESVQSNNSSSFLNFGFANRFSKPKGPRNPPPTWN |
| | I(SEQ ID NO: 10) |
| Zap70 SH2 1, | MPDPAAHLPFFYGSISRAEAEEHLKLAGMADGLFLLRQCLRSLGGYVLSLVHDVRF |
| SH2 2 domains | HHFPIERQLNGTYAIAGGKAHCGPAELCEFYSRDPDGLPCNLRKPCNRPSGLEPQPG |
| | VFDCLRDAMVRDYVRQTWKLEGEALEQAIISQAPQVEKLIATTAHERMPWYHSSL |
| | TREEAERKLYSGAQTDGKFLLRPRKEQGTYALSLIYGKTVYHYLISQDKAGKYCIPE |
| | GTKFDTLWQLVEYLKLKADGLIYCLKEAC (SEQ ID NO: 11) |
| Zap70 delta | MPDPAAHLPFFYGSISRAEAEEHLKLAGMADGLFLLRQCLRSLGGYVLSLVHDVRF |
| kinase | HHFPIERQLNGTYAIAGGKAHCGPAELCEFYSRDPDGLPCNLRKPCNRPSGLEPQPG |
| | VFDCLRDAMVRDYVRQTWKLEGEALEQAIISQAPQVEKLIATTAHERMPWYHSSL |
| | TREEAERKLYSGAQTDGKFLLRPRKEQGTYALSLIYGKTVYHYLISQDKAGKYCIPE |
| | GTKFDTLWQLVEYLKLKADGLIYCLKEACPNSSASNASGAAAPTLPAHPSTLTHPQ |
| | RRIDTLNSDGYTPEPARITSPDKPRPMPMDTSVYESPYSDPEELKDKKLFLKRDNL |
| | (SEQ ID NO: 12) |
| Zap70 Y492F | MPDPAAHLPFFYGSISRAEAEEHLKLAGMADGLFLLRQCLRSLGGYVLSLVHDVRF |
| Y493F | HHFPIERQLNGTYAIAGGKAHCGPAELCEFYSRDPDGLPCNLRKPCNRPSGLEPQPG |
| | VFDCLRDAMVRDYVRQTWKLEGEALEQAIISQAPQVEKLIATTAHERMPWYHSSL |
| | TREEAERKLYSGAQTDGKFLLRPRKEQGTYALSLIYGKTVYHYLISQDKAGKYCIPE |
| | GTKFDTLWQLVEYLKLKADGLIYCLKEACPNSSASNASGAAAPTLPAHPSTLTHPQ |
| | RRIDTLNSDGYTPEPARITSPDKPRPMPMDTSVYESPYSDPEELKDKKLFLKRDNLLI |
| | ADIELGCGNFGSVRQGVYRMRKKQIDVAIKVLKQGTEKADTEEMMREAQIMHQLD |
| | NPYIVRLIGVCQAEALMLVMEMAGGGPLHKFLVGKREEIPVSNVAELLHQVSMGM |
| | KYLEEKNFVHRDLAARNVLLVNRHYAKISDFGLSKALGADDSFFTARSAGKWPLK |
| | WYAPECINFRKFSSRSDVWSYGVTMWEALSYGQKPYKKMKGPEVMAFIEQGKRM |
| | ECPPECPPELYALMSDCWIYKWEDRPDFLTVEQRMRACYYSLASKVEGPPGSTQKA |
| | EAACA (SEQ ID NO: 13) |
| SHIP-1 PTP | DMITIFIGTWNMGNAPPPKKITSWFLSKGQGKTRDDSADYIPHDIYVIGTQEDPLSEK |
| domain | EWLEILKHSLQEITSVTFKTVAIHTLWNIRIVVLAKPEHENRISHICTDNVKTGIANTL |
| | GNKGAVGVSFMFNGTSLGFVNSHLTSGSEKKLRRNQNYMNILRFLALGDKKLSPFN |
| | ITHRFTHLFWFGDLNYRVDLPTWEAETIIQKIKQQQYADLLSHDQLLTERREQKVFL |
| | HFEEEEITFAPTYRFERLTRDKYAYTKQKATGMKYNLPSWCDRVLWKSYPLVHVV |
| | CQSYGSTSDIMTSDHSPVFATFEAGVTSQFVS (SEQ ID NO: 14) |

TABLE 1B
Enzymatic Inhibitory Domains Nucleic Acid Sequences

| Domain | Nucleic Acid Sequence |
|---|---|
| Csk | AGTGCCATTCAGGCTGCATGGCCGTCAGGTACGGAGTGTATTGCTAAATACAAT |
| | TTTCACGGGACTGCTGAACAGGACCTTCCTTTTTGCAAAGGGGACGTCTTGACT |
| | ATCGTAGCTGTGACGAAAGACCCGAACTGGTACAAAGCTAAGAATAAGGTCGG |
| | CCGGGAGGGTATAATTCCCGCAAATTACGTGCAGAAGCGAGAAGGTGTTAAAG |
| | CTGGAACAAAGTTGTCACTCATGCCGTGGTTTCATGGAAAAATTACCAGAGAAC |
| | AAGCGGAGCGGCTGTTGTACCCGCCGGAAACCGGCCTTTTCTTGGTTAGGGAAA |
| | GCACCAATTACCCTGGGGACTACACTCTTTGTGTTTCATGCGATGGGAAGGTAG |
| | AACATTACCGCATCATGTATCATGCCAGTAAGCTTTCCATAGACGAAGAGGTGT |
| | ACTTCGAGAACCTGATGCAACTGGTGGAGCACTACACATCCGATGCGGACGGAT |
| | TGTGCACGCGATTGATAAAACCAAAGGTAATGGAAGGCACCGTGGCAGCTCAG |
| | GATGAGTTTTACCGAAGCGGTTGGGCACTGAACATGAAAGAACTTAAACTGCTT |
| | CAGACAATTGGGAAAGGAGAATTTGGCGACGTGATGCTGGGTGATTATAGGGG |
| | TAACAAGGTCGCGGTGAAGTGCATTAAGAACGATGCCACGGCCCAGGCCTTTCT |
| | GGCAGAAGCTTCAGTTATGACTCAACTCCGCCATTCTAATCTGGTCCAATTGCTT |
| | GGAGTGATCGTCGAAGAGAAGGGAGGCCTCTACATAGTAACGGAGTATATGGC |
| | TAAAGGTTCTCTCGTCGATTATCTCCGCTCACGCGGGCGCAGCGTTCTCGGGGG |
| | AGATTGCCTCCTGAAATTCAGTCTGGACGTCTGCGAGGCTATGGAGTACCTCGA |
| | AGGAAACAATTTTGTCCACCGGGACTTGGCTGCAAGGAACGTCCTGGTAAGCGA |
| | AGATAACGTGGCTAAGGTGTCCGACTTCGGGCTCACGAAAGAAGCAAGTAGTA |
| | CTCAAGACACGGGCAAACTCCCAGTTAAATGGACCGCACCGGAGGCACTCAGG |
| | GAAAAAAAATTTTCTACCAAGAGTGACGTATGGTCATTCGGCATCCTCTTGTGG |
| | GAAATATATAGTTTTGGGCGCGTTCCTTACCCAAGAATCCCCCTGAAGGATGTC |
| | GTGCCGAGAGTGGAAAAGGGGTACAAGATGGATGCTCCGGACGGATGCCCCCC |
| | AGCAGTATATGAGGTAATGAAAAATTGCTGGCATCTCGATGCAGCGATGCGGCC |
| | GTCCTTTCTTCAGCTCCGGGAGCAACTGGAACACATAAAGACGCACGAACTTCA |
| | TCTT (SEQ ID NO: 15) |
| Csk de1taSH3 | GTTAAAGCTGGAACAAAGTTGTCACTCATGCCGTGGTTTCATGGAAAAATTACC |
| | AGAGAACAAGCGGAGCGGCTGTTGTACCCGCCGGAAACCGGCCTTTTCTTGGTT |
| | AGGGAAAGCACCAATTACCCTGGGGACTACACTCTTTGTGTTTCATGCGATGGG |
| | AAGGTAGAACATTACCGCATCATGTATCATGCCAGTAAGCTTTCCATAGACGAA |
| | GAGGTGTACTTCGAGAACCTGATGCAACTGGTGGAGCACTACACATCCGATGCG |
| | GACGGATTGTGCACGCGATTGATAAAACCAAAGGTAATGGAAGGCACCGTGGC |
| | AGCTCAGGATGAGTTTTACCGAAGCGGTTGGGCACTGAACATGAAAGAACTTAA |
| | ACTGCTTCAGACAATTGGGAAAGGAGAATTTGGCGACGTGATGCTGGGTGATTA |
| | TAGGGGTAACAAGGTCGCGGTGAAGTGCATTAAGAACGATGCCACGGCCCAGG |
| | CCTTTCTGGCAGAAGCTTCAGTTATGACTCAACTCCGCCATTCTAATCTGGTCCA |
| | ATTGCTTGGAGTGATCGTCGAAGAGAAGGGAGGCCTCTACATAGTAACGGAGT |
| | ATATGGCTAAAGGTTCTCTCGTCGATTATCTCCGCTCACGCGGGCGCAGCGTTCT |
| | CGGGGGAGATTGCCTCCTGAAATTCAGTCTGGACGTCTGCGAGGCTATGGAGTA |
| | CCTCGAAGGAAACAATTTTGTCCACCGGGACTTGGCTGCAAGGAACGTCCTGGT |
| | AAGCGAAGATAACGTGGCTAAGGTGTCCGACTTCGGGCTCACGAAAGAAGCAA |
| | GTAGTACTCAAGACACGGGCAAACTCCCAGTTAAATGGACCGCACCGGAGGCA |
| | CTCAGGGAAAAAAAATTTTCTACCAAGAGTGACGTATGGTCATTCGGCATCCTC |
| | TTGTGGGAAATATATAGTTTTGGGCGCGTTCCTTACCCAAGAATCCCCCTGAAG |
| | GATGTCGTGCCGAGAGTGGAAAAGGGGTACAAGATGGATGCTCCGGACGGATG |
| | CCCCCCAGCAGTATATGAGGTAATGAAAAATTGCTGGCATCTCGATGCAGCGAT |
| | GCGGCCGTCCTTTCTTCAGCTCCGGGAGCAACTGGAACACATAAAGACGCACGA |
| | ACTTCATCTT (SEQ ID NO: 16) |
| SHP-1 | ATGGTTCGATGGTTCCACAGAGATCTGAGCGGCCTGGATGCCGAGACTCTGCTT |
| | AAAGGACGGGGCGTGCACGGCAGCTTTCTGGCTAGACCCAGCAGAAAGAACCA |
| | GGGCGACTTCAGCCTGTCCGTCAGAGTGGGCGATCAAGTGACACACATCAGAAT |
| | CCAGAACTCCGGCGACTTCTACGACCTGTACGGCGGCGAGAAGTTCGCCACACT |
| | GACAGAGCTGGTGGAATATTACACCCAGCAGCAAGGCGTGCTGCAGGACAGAG |
| | ATGGCACCATCATCCACCTGAAGTACCCTCTGAACTGCAGCGACCCCACCAGCG |
| | AGAGATGGTATCACGGACACATGTCTGGCGGCCAGGCTGAGACACTGCTTCAGG |
| | CTAAAGGCGAGCCCTGGACCTTTCTCGTGCGGGAATCTCTGAGTCAGCCCGGCG |
| | ATTTTGTGCTGAGCGTGCTGTCCGATCAGCCCAAAGCTGGACCAGGCTCTCCAC |
| | TGAGAGTGACCCATATCAAAGTGATGTGCGAAGGCGGACGGTACACCGTCGGT |
| | GGCCTGGAAACATTCGATAGCCTGACCGACCTGGTCGAGCACTTCAAGAAAACC |
| | GGCATCGAGGAAGCCAGCGGCGCCTTCGTTTATCTGAGACAGCCCTACTACGCC |
| | ACAAGAGTGAACGCCGCCGACATCGAGAACAGAGTGCTGGAACTGAACAAGAA |
| | GCAAGAGAGCGAGGATACCGCCAAGGCCGGCTTCTGGGAAGAGTTCGAGTCCC |
| | TGCAGAAACAAGAAGTGAAGAACCTGCACCAGCGGCTGGAAGGACAGAGGCCT |
| | GAGAACAAGGGCAAGAACCGGTACAAGAACATCCTGCCATTCGACCACTCCAG |
| | AGTGATCCTGCAGGGAAGAGACAGCAACATCCCCGGCTCCGACTACATCAACG |
| | CCAATTACATCAAGAACCAGCTGCTGGGCCCCGACGAGAACGCCAAGACATAT |
| | ATCGCCTCTCAGGGCTGCCTGGAAGCCACCGTGAACGACTTTTGGCAGATGGCC |
| | TGGCAAGAGAACAGCCGCGTGATCGTGATGACCACCAGAGAGGTGGAAAAAGG |
| | CCGGAACAAATGCGTGCCCTACTGGCCCGAAGTGGGCATGCAAAGAGCCTACG |
| | GACCTTACAGCGTGACCAACTGCGGCGAGCACGATACCACCGAGTACAAGCTG |
| | AGAACCCTCCAGGTGTCCCCTCTGGACAACGGCGACCTGATCAGAGAGATCTGG |
| | CACTACCAGTACCTGTCCTGGCCTGATCACGGCGTGCCATCTGAACCTGGTGGC |
| | GTGCTGAGTTTCCTGGACCAGATCAACCAGAGACAAGAGAGCCTGCCTCACGCT |
| | GGCCCTATCATCGTGCACTGTTCTGCCGGCATCGGCAGAACCGGCACAATCATC |
| | GTGATCGACATGCTGATGGAAAACATCAGCACCAAGGGCCTCGACTGCGACATC |
| | GACATCCAGAAAACCATCCAGATGGTTCGAGCCCAGCGGAGCGGAATGGTGCA |
| | GACAGAAGCCCAGTACAAGTTCATCTACGTGGCAATCGCCCAGTTCATCGAAAC |
| | CACCAAGAAAAAGCTGGAAGTGCTGCAGTCCCAGAAGGGCCAAGAAAGCGAGT |
| | ACGGCAACATCACATACCCTCCAGCCATGAAGAACGCCCACGCCAAAGCCAGC |
| | AGAACCTCCAGCAAGCACAAAGAGGACGTCTACGAGAATCTGCACACAAAGAA |
| | CAAGCGCGAGGAAAAAGTGAAAAAGCAGCGCAGCGCCGACAAAGAGAAGTCC |
| | AAGGGCAGCCTGAAGAGAAAG (SEQ ID NO: 17) |
| SHP-1 PTP | TTCTGGGAAGAGTTCGAGTCCCTGCAGAAACAAGAAGTGAAGAACCTGCACCA |
| domain | GCGGCTGGAAGGACAGAGGCCTGAGAACAAGGGCAAGAACCGGTACAAGAAC |
| | ATCCTGCCATTCGACCACTCCAGAGTGATCCTGCAGGGAAGAGACAGCAACATC |
| | CCCGGCTCCGACTACATCAACGCCAATTACATCAAGAACCAGCTGCTGGGCCCC |
| | GACGAGAACGCCAAGACATATATCGCCTCTCAGGGCTGCCTGGAAGCCACCGTG |
| | AACGACTTTTGGCAGATGGCCTGGCAAGAGAACAGCCGCGTGATCGTGATGACC |
| | ACCAGAGAGGTGGAAAAAGGCCGGAACAAATGCGTGCCCTACTGGCCCGAAGT |
| | GGGCATGCAAAGAGCCTACGGACCTTACAGCGTGACCAACTGCGGCGAGCACG |
| | ATACCACCGAGTACAAGCTGAGAACCCTCCAGGTGTCCCCTCTGGACAACGGCG |
| | ACCTGATCAGAGAGATCTGGCACTACCAGTACCTGTCCTGGCCTGATCACGGCG |
| | TGCCATCTGAACCTGGTGGCGTGCTGAGTTTCCTGGACCAGATCAACCAGAGAC |
| | AAGAGAGCCTGCCTCACGCTGGCCCTATCATCGTGCACTGTTCTGCCGGCATCG |
| | GCAGAACCGGCACAATCATCGTGATCGACATGCTGATGGAAAACATCAGCACC |
| | AAGGGCCTCGACTGCGACATCGACATCCAGAAAACCATCCAGATGGTTCGAGCC |
| | CAGCGGAGCGGAATGGTGCAGACAGAAGCCCAGTACAAGTTCATCTACGTGGC |
| | AATCGCCCAGTTC (SEQ ID NO: 18) |
| SHP-2 | ATGACAAGCAGACGGTGGTTTCACCCCAACATCACCGGCGTGGAAGCTGAGAA |
| | TCTGCTGCTGACAAGAGGCGTGGACGGCAGCTTTCTGGCTAGACCCAGCAAGTC |
| | CAATCCTGGCGACTTCACACTGAGCGTGCGGAGAAATGGCGCCGTGACACACAT |
| | CAAGATCCAGAACACCGGCGACTACTACGACCTGTACGGCGGCGAGAAGTTTG |
| | CCACACTGGCAGAGCTGGTGCAGTACTACATGGAACACCACGGCCAGCTGAAA |
| | GAAAAGAACGGCGACGTGATCGAGCTGAAGTACCCTCTGAACTGCGCCGATCCT |
| | ACCAGCGAGAGATGGTTTCACGGCCACCTGTCTGGCAAAGAGGCCGAGAAGCT |
| | GCTGACCGAGAAGGGCAAGCACGGAAGCTTTCTCGTGCGCGAGTCTCAGTCTCA |
| | CCCCGGCGATTTTGTGCTGTCTGTGCGGACAGGGGACGACAAGGGCGAGAGCA |
| | ATGACGGCAAGAGCAAAGTGACCCACGTGATGATCCGGTGCCAAGAGCTGAAA |
| | TACGACGTCGGCGGAGGGGAGAGATTCGACTCTCTGACCGATCTGGTGGAACAC |
| | TACAAGAAAAACCCCATGGTGGAAACCCTGGGCACCGTGCTGCAGCTGAAGCA |
| | GCCACTGAACACCACCAGAATCAACGCCGCCGAGATCGAGAGCAGAGTGCGGG |
| | AACTGTCTAAGCTGGCCGAGACTACCGACAAAGTGAAGCAAGGCTTCTGGGAA |
| | GAGTTCGAGACACTGCAGCAGCAAGAGTGCAAGCTGCTGTACTCCCGGAAAGA |
| | GGGCCAGAGACAAGAGAACAAGAACAAAAACCGGTACAAGAACATCCTGCCGT |
| | TCGATCACACCAGAGTGGTGCTGCACGACGGCGATCCTAATGAGCCCGTGTCCG |
| | ACTACATCAACGCCAACATCATCATGCCCGAGTTTGAGACAAAGTGCAACAATA |
| | GCAAGCCCAAGAAGTCCTATATCGCCACACAGGGCTGCCTGCAGAATACCGTGA |
| | ACGACTTTTGGCGGATGGTGTTTCAAGAGAACTCCCGCGTGATCGTGATGACCA |
| | CCAAAGAGGTGGAACGGGGCAAGTCTAAGTGCGTGAAGTACTGGCCCGACGAG |
| | TACGCCCTGAAAGAATACGGCGTGATGAGAGTGCGGAACGTGAAAGAGAGCGC |
| | CGCTCACGATTACACCCTGAGAGAGCTGAAGCTGAGCAAAGTCGGACAGGCCC |
| | TGCTGCAGGGAAACACCGAAAGAACCGTGTGGCAGTACCACTTCCGGACCTGG |
| | CCAGATCATGGCGTGCCATCTGATCCTGGCGGCGTGCTGGATTTCCTGGAAGAG |
| | GTGCACCACAAGCAAGAGTCCATCATGGACGCCGGACCAGTGGTGGTGCACTGT |
| | TCTGCCGGAATCGGAAGAACCGGCACCTTCATCGTGATCGACATCCTGATTGAC |
| | ATCATCCGCGAGAAAGGCGTCGACTGCGATATCGACGTGCCCAAGACCATCCAG |
| | ATGGTTCGAAGCCAGAGAAGCGGCATGGTGCAGACAGAGGCCCAGTACCGGTT |
| | CATCTACATGGCCGTGCAGCATTACATCGAAACCCTGCAGCGGCGGATCGAGGA |
| | AGAACAGAAGTCCAAGAGAAAGGGCCACGAGTACACCAACATCAAGTACAGCC |
| | TGGCCGACCAGACCAGCGGCGATCAATCTCCTCTGCCTCCTTGCACACCCACAC |
| | CTCCATGTGCCGAGATGCGGGAAGATAGCGCCAGGGTGTACGAGAACGTGGGC |
| | CTGATGCAACAGCAGAAGTCCTTCCGG (SEQ ID NO: 19) |
| CD45 | TACAAGATCTACGACCTGCACAAGAAGCGGAGCTGCAATCTGGACGAGCAGCA |
| | AGAACTGGTGGAACGGGACGACGAGAAGCAGCTGATGAACGTGGAACCCATCC |
| | ACGCCGACATCCTGCTGGAAACCTACAAGCGGAAGATCGCCGACGAGGGCAGA |
| | CTGTTCCTGGCCGAGTTTCAGAGCATCCCCAGAGTGTTCAGCAAGTTCCCCATCA |
| | AAGAGGCCAGAAAGCCCTTCAACCAGAACAAGAACCGCTACGTGGACATTCTG |
| | CCCTACGACTACAATCGCGTGGAACTGAGCGAGATCAATGGCGACGCCGGCAG |
| | CAACTACATCAACGCCAGCTACATCGACGGCTTCAAAGAGCCCCGGAAGTATAT |
| | CGCCGCTCAGGGCCCTAGAGATGAGACAGTGGACGACTTCTGGCGCATGATTTG |
| | GGAGCAGAAAGCCACCGTGATCGTGATGGTTACCAGATGCGAAGAGGGCAACA |
| | GAAACAAGTGCGCCGAGTACTGGCCCAGCATGGAAGAAGGCACAAGAGCCTTT |
| | GGCGACGTGGTGGTCAAGATCAATCAGCACAAGCGGTGCCCCGACTACATCATC |
| | CAGAAACTGAACATCGTGAACAAGAAAGAGAAGGCCACCGGACGGGAAGTGA |
| | CCCACATCCAGTTTACCAGCTGGCCCGATCATGGCGTGCCCGAGGATCCACATC |
| | TGCTGCTCAAGCTGCGGAGAAGAGTGAACGCCTTCAGCAACTTCTTCAGCGGCC |
| | CCATCGTGGTGCACTGTTCTGCAGGCGTTGGAAGAACCGGCACCTACATCGGAA |
| | TCGACGCCATGCTGGAAGGACTGGAAGCCGAGAACAAGGTGGACGTGTACGGC |
| | TACGTGGTCAAGCTGAGAAGGCAGCGGTGTCTGATGGTGCAGGTTGAGGCCCA |
| | GTACATCCTGATCCATCAGGCCCTGGTCGAGTACAACCAGTTCGGCGAGACAGA |
| | AGTGAACCTGAGCGAGCTGCACCCCTATCTGCACAACATGAAGAAGCGGGACC |
| | CTCCAAGCGAGCCCTCTCCACTGGAAGCTGAGTTCCAGAGACTGCCCAGCTACA |
| | GAAGCTGGCGGACACAGCACATCGGCAATCAAGAGGAAAACAAGAGCAAGAA |
| | CCGGAACAGCAACGTGATCCCGTACGATTACAACAGAGTGCCCCTGAAGCACG |
| | AACTCGAGATGAGCAAAGAGAGCGAGCACGACAGCGACGAGTCCAGCGACGAT |
| | GATAGCGACAGCGAGGAACCCAGCAAGTATATCAATGCCTCCTTCATCATGAGC |
| | TATTGGAAGCCCGAAGTGATGATTGCCGCACAGGGACCCCTGAAAGAGACAAT |
| | CGGCGACTTTTGGCAGATGATCTTCCAGCGGAAAGTGAAAGTGATCGTCATGCT |
| | GACCGAGCTGAAACACGGCGACCAAGAGATCTGCGCCCAGTATTGGGGAGAGG |
| | GAAAGCAGACCTACGGCGACATTGAGGTGGACCTGAAGGACACCGACAAGAGC |
| | AGCACCTACACACTGCGGGTGTTCGAGCTGAGACACTCCAAGAGAAAGGACAG |
| | CCGGACCGTGTACCAGTACCAGTATACCAATTGGAGCGTGGAACAGCTGCCTGC |
| | CGAGCCTAAAGAACTGATCAGCATGATCCAGGTCGTGAAGCAGAAGCTGCCTC |
| | AGAAGAACAGCAGCGAGGGAAACAAGCACCACAAGTCTACCCCTCTGCTGATC |
| | CACTGCAGAGATGGCTCTCAGCAGACCGGCATCTTCTGCGCCCTGCTGAATCTC |
| | CTGGAAAGCGCCGAGACAGAGGAAGTGGTGGACATCTTCCAGGTGGTCAAAGC |
| | CCTGCGGAAGGCCAGACCTGGCATGGTGTCTACCTTCGAGCAGTATCAGTTCCT |
| | GTACGACGTGATCGCCAGCACATACCCCGCTCAGAACGGCCAAGTGAAGAAGA |
| | ACAACCACCAAGAGGACAAGATCGAGTTCGACAACGAGGTGGACAAAGTGAAG |
| | CAGGACGCCAACTGCGTGAACCCTCTGGGAGCCCCAGAAAAGCTGCCTGAGGC |
| | CAAAGAACAGGCCGAGGGCTCTGAGCCAACATCTGGAACAGAGGGACCTGAGC |
| | ACAGCGTGAACGGACCTGCTAGCCCCGCTCTGAATCAGGGCTCT (SEQ ID |
| | NO: 20) |
| Grb2 | ATGGAAGCCATTGCCAAATACGACTTCAAGGCCACCGCCGACGACGAGCTGAG |
| | CTTCAAGAGAGGCGACATCCTGAAGGTGCTGAACGAGGAATGCGACCAGAACT |
| | GGTACAAGGCCGAGCTGAACGGCAAGGACGGCTTCATCCCCAAGAACTACATC |
| | GAGATGAAGCCCCATCCATGGTTCTTCGGCAAGATCCCCAGAGCCAAGGCCGAA |
| | GAGATGCTGAGCAAGCAGAGACACGACGGCGCCTTTCTGATCCGGGAATCTGA |
| | ATCTGCCCCTGGCGACTTCAGCCTGTCCGTGAAGTTCGGCAACGACGTGCAGCA |
| | CTTCAAGGTCCTGAGAGATGGCGCCGGAAAGTACTTCCTGTGGGTCGTGAAGTT |
| | TAACAGCCTGAACGAGCTGGTGGACTACCACAGATCTACCAGCGTGTCCCGGAA |
| | CCAGCAGATCTTCCTGCGGGACATCGAACAGGTTCCCCAGCAACCTACCTACGT |
| | GCAGGCCCTGTTCGACTTCGACCCTCAAGAGGATGGCGAGCTGGGCTTTAGACG |
| | GGGCGATTTCATCCACGTGATGGACAATAGCGACCCCAACTGGTGGAAGGGCG |
| | CCTGTCATGGACAGACCGGCATGTTCCCCAGAAACTACGTGACCCCTGTGAACC |
| | GGAACGTG (SEQ ID NO: 21) |
| PTEN | ATGACAGCCATCATCAAAGAAATCGTGTCCCGGAACAAGCGGCGCTACCAAGA |
| | GGATGGCTTCGACCTGGACCTGACCTACATCTACCCCAACATCATTGCCATGGG |
| | CTTCCCCGCCGAAAGACTGGAAGGCGTGTACAGAAACAACATCGACGATGTCGT |
| | GCGGTTCCTGGACAGCAAGCACAAGAACCACTACAAGATCTACAACCTGTGCGC |
| | CGAGCGGCACTACGATACCGCCAAGTTCAACTGCAGAGTGGCTCAGTACCCCTT |
| | CGAGGACCACAATCCTCCACAGCTGGAACTGATCAAGCCCTTCTGCGAGGACCT |
| | GGATCAGTGGCTGAGCGAGGACGATAATCACGTGGCCGCCATTCACTGCAAGG |
| | CCGGCAAGGGAAGAACCGGCGTGATGATCTGTGCCTACCTGCTGCACCGGGGC |
| | AAGTTTCTGAAAGCCCAAGAGGCCCTGGACTTCTACGGCGAAGTGCGGACCAG |
| | AGACAAGAAAGGCGTGACAATCCCCAGCCAGCGGAGATACGTGTACTACTACA |
| | GCTATCTGCTGAAGAACCACCTGGACTACAGACCCGTGGCACTGCTGTTCCACA |
| | AGATGATGTTCGAGACTATCCCCATGTTCAGCGGCGGCACATGCAACCCTCAGT |
| | TCGTCGTGTGCCAGCTGAAAGTGAAGATCTACTCCAGCAACAGCGGCCCCACCA |
| | GACGCGAGGACAAGTTCATGTACTTCGAGTTCCCTCAGCCTCTGCCTGTGTGCG |
| | GCGACATCAAGGTGGAATTCTTCCACAAGCAGAACAAGATGCTGAAAAAGGAC |
| | AAGATGTTCCACTTCTGGGTCAACACCTTCTTCATCCCCGGACCTGAAGAGACA |
| | AGCGAGAAGGTGGAAAACGGCAGCCTGTGCGACCAAGAGATCGACAGCATCTG |
| | CAGCATCGAGCGGGCCGACAACGACAAAGAATACCTGGTGCTGACCCTGACCA |
| | AGAACGATCTGGACAAGGCCAACAAGGATAAGGCCAACCGGTACTTCAGCCCC |
| | AACTTCAAAGTGAAACTGTACTTCACCAAGACCGTCGAGGAACCCAGCAATCCT |
| | GAGGCCAGCTCTAGCACATCTGTGACCCCTGACGTGTCCGACAATGAGCCCGAC |
| | CACTACAGATACAGCGACACCACCGACAGCGACCCCGAGAACGAGCCTTTCGA |
| | TGAGGATCAGCACACACAGATCACCAAAGTG (SEQ ID NO: 22) |
| PTP-MEG1 | ATGACAAGCAGATTCAGACTGCCAGCCGGCAGAACCTACAATGTGCGGGCTTCT |
| | GAGCTGGCCAGAGACAGACAGCACACCGAGGTCGTGTGCAACATCCTGCTGCTC |
| | GACAATACCGTGCAGGCCTTCAAAGTGAACAAGCACGACCAGGGCCAAGTCCT |
| | GCTGGACGTGGTGTTCAAGCACCTGGATCTGACCGAGCAGGACTACTTCGGACT |
| | GCAGCTGGCCGACGACAGCACCGACAATCCTAGATGGCTGGACCCCAACAAGC |
| | CCATCCGGAAGCAGCTGAAGAGAGGCTCCCCTTACTCTCTGAACTTCCGGGTCA |
| | AGTTCTTCGTGTCCGATCCTAACAAACTGCAAGAAGAGTATACCCGCTACCAGT |
| | ACTTCCTGCAGATCAAGCAGGACATCCTGACCGGCAGACTGCCCTGTCCTTCTA |
| | ATACTGCCGCTCTGCTGGCCTCCTTTGCCGTGCAATCTGAACTGGGCGACTACGA |
| | CCAGAGCGAGAACCTGAGCGGCTACCTGAGCGATTACAGCTTCATCCCCAACCA |
| | GCCTCAGGACTTCGAGAAAGAGATCGCCAAGCTGCATCAGCAGCACATCGGAC |
| | TGTCTCCAGCCGAGGCCGAGTTCAACTACCTGAACACCGCCAGGACACTGGAAC |
| | TGTACGGCGTGGAATTTCACTACGCCCGGGACCAGAGCAACAACGAGATCATG |
| | ATTGGCGTGATGAGCGGCGGCATCCTGATCTACAAGAACAGAGTGCGGATGAA |
| | CACATTCCCCTGGCTGAAGATCGTGAAGATCAGCTTCAAGTGCAAGCAGTTCTT |
| | CATCCAGCTGCGGAAAGAGCTGCACGAGAGCAGAGAGACACTGCTGGGCTTCA |
| | ACATGGTCAACTACCGGGCCTGCAAGAACCTGTGGAAGGCCTGTGTGGAACACC |
| | ACACCTTTTTCCGGCTGGACCGACCTCTGCCTCCTCAGAAGAATTTCTTCGCCCA |
| | CTACTTCACCCTGGGCAGCAAGTTCAGATACTGCGGCAGAACAGAGGTGCAGTC |
| | CGTGCAGTACGGCAAAGAGAAAGCCAACAAGGACCGGGTGTTCGCCAGATCTC |
| | CTAGCAAGCCACTGGCCAGAAAGCTGATGGACTGGGAAGTCGTGTCCCGGAAC |
| | AGCATCAGCGACGACAGACTGGAAACCCAGAGCCTGCCTAGCAGAAGCCCTCC |
| | TGGCACACCCAACCACAGAAACAGCACCTTCACACAAGAGGGCACAAGACTGA |
| | GGCCTAGCTCTGTGGGACACCTGGTGGATCACATGGTGCACACAAGCCCCAGCG |
| | AGGTGTTCGTGAACCAGAGAAGCCCTAGCTCTACCCAGGCCAACAGCATCGTGC |
| | TGGAAAGCAGCCCCAGCCAAGAAACACCAGGCGACGGAAAACCTCCTGCTCTG |
| | CCACCTAAGCAGAGCAAGAAGAACAGCTGGAACCAGATCCACTACAGCCACAG |
| | CCAGCAGGATCTGGAAAGCCACATCAACGAGACATTCGACATCCCTAGCAGCCC |
| | CGAGAAGCCCACACCTAATGGCGGAATCCCTCACGACAACCTGGTGCTGATCCG |
| | GATGAAGCCCGACGAGAATGGCAGATTCGGCTTCAACGTGAAAGGCGGCTACG |
| | ATCAGAAAATGCCCGTGATCGTGTCCAGAGTGGCCCCTGGAACTCCTGCCGATC |
| | TGTGTGTGCCCAGACTGAACGAAGGCGACCAGGTCGTGCTGATCAACGGCAGA |
| | GATATCGCCGAGCACACCCACGATCAGGTGGTGCTGTTCATTAAGGCCTCTTGC |
| | GAGAGACACAGCGGCGAACTGATGCTGCTCGTGCGGCCTAATGCCGTGTACGAC |
| | GTGGTGGAAGAGAAACTGGAAAACGAGCCCGACTTCCAGTACATCCCTGAGAA |
| | GGCCCCACTGGACAGCGTGCACCAGGATGATCATAGCCTGCGCGAGAGCATGA |
| | TCCAGCTGGCAGAGGGACTGATCACCGGCACAGTGCTGACCCAGTTCGACCAGC |
| | TGTACCGGAAGAAACCTGGCATGACCATGTCCTGCGCCAAACTGCCTCAGAACA |
| | TCAGCAAGAACCGGTACAGAGACATCAGCCCCTACGATGCCACCAGAGTGATC |
| | CTGAAGGGCAACGAGGACTACATCAACGCCAATTACATCAACATGGAAATCCC |
| | CAGCTCCAGCATCATCAACCAGTATATCGCCTGTCAGGGCCCACTGCCTCACAC |
| | CTGTACCGACTTTTGGCAGATGACCTGGGAGCAGGGCAGCAGCATGGTGGTCAT |
| | GCTGACAACCCAGGTGGAACGGGGCAGAGTGAAGTGCCACCAGTATTGGCCTG |
| | AGCCTACCGGCAGCAGCTCCTACGGCTGTTACCAAGTGACCTGCCACAGCGAAG |
| | AGGGCAACACCGCCTACATCTTCAGAAAGATGACCCTGTTCAATCAAGAGAAG |
| | AACGAGAGCCGGCCTCTGACACAGATCCAGTATATTGCTTGGCCCGACCACGGC |
| | GTGCCCGACGATAGTTCTGACTTCCTGGACTTCGTGTGCCACGTGCGCAACAAA |
| | CGCGCCGGAAAAGAGGAACCTGTCGTCGTCCACTGTAGCGCCGGCATTGGAAG |
| | AACCGGCGTGCTGATTACCATGGAAACAGCCATGTGCCTGATCGAGTGCAATCA |
| | GCCCGTGTATCCCCTGGACATCGTGCGGACCATGAGAGATCAGCGGGCCATGAT |
| | GATCCAGACACCTAGCCAGTACAGATTCGTGTGCGAGGCCATTCTGAAGGTGTA |
| | CGAAGAGGGATTCGTGAAGCCCCTGACCACCTCCACCAACAAG (SEQ ID |
| | NO: 23) |
| PTPN22 | GATCAGAGAGAGATCCTGCAGAAGTTCCTGGACGAGGCCCAGAGCAAGAAGAT |
| | CACCAAAGAGGAATTCGCCAACGAGTTCCTGAAACTGAAGCGGCAGAGCACCA |
| | AGTACAAGGCCGACAAGACATACCCCACCACCGTGGCCGAGAAGCCCAAGAAC |
| | ATCAAGAAGAACCGGTACAAGGACATCCTGCCATACGACTACTCCAGAGTGGA |
| | ACTGAGCCTGATCACCAGCGACGAGGACAGCAGCTACATCAACGCCAACTTCAT |
| | CAAGGGCGTGTACGGCCCCAAGGCCTATATCGCAACACAGGGCCCTCTGTCTAC |
| | AACCCTGCTGGACTTCTGGCGCATGATCTGGGAGTACAGCGTGCTGATCATCGT |
| | GATGGCCTGCATGGAATACGAGATGGGCAAGAAGAAGTGCGAGCGGTACTGGG |
| | CCGAACCTGGCGAAATGCAGCTGGAATTCGGCCCCTTTTCCGTGTCCTGCGAAG |
| | CCGAGAAGAGAAAGTCCGACTACATCATCAGGACCCTGAAAGTGAAGTTCAAC |
| | AGCGAAACCCGGACCATCTACCAGTTTCACTACAAGAACTGGCCCGACCACGAC |
| | GTGCCAAGCAGCATCGATCCTATCCTGGAACTGATTTGGGACGTGCGGTGCTAC |
| | CAAGAGGACGACAGCGTGCCAATCTGCATCCACTGTTCTGCCGGCTGCGGAAGA |
| | ACAGGCGTCATCTGCGCCATCGACTACACCTGGATGCTGCTGAAGGACGGCATC |
| | ATCCCCGAGAACTTCAGCGTGTTCAGCCTGATCCGCGAGATGAGAACCCAGAGG |
| | CCTAGCCTGGTGCAGACCCAAGAGCAGTACGAACTGGTGTACAACGCCGTGCTG |
| | GAACTGTTCAAGAGACAGATGGACGTGATCCGGGACAAGCACAGCGGCACAGA |
| | GTCTCAGGCCAAACACTGCATCCCTGAGAAGAATCACACCCTGCAGGCCGACAG |
| | CTACAGCCCCAATCTGCCTAAGAGCACCACCAAGGCCGCCAAAATGATGAACC |
| | AGCAGCGGACAAAGATGGAAATCAAAGAGAGCAGCTCCTTCGACTTCCGGACC |
| | AGCGAGATCAGCGCCAAAGAAGAACTGGTTCTGCACCCCGCCAAGTCCTCTACC |
| | AGCTTCGACTTTCTCGAGCTGAACTACAGCTTCGATAAGAACGCCGACACCACC |
| | ATGAAGTGGCAGACCAAGGCCTTTCCTATCGTGGGCGAGCCTCTGCAGAAACAC |
| | CAGAGCCTGGATCTGGGCTCCCTGCTGTTTGAGGGCTGCAGCAATAGCAAGCCC |
| | GTGAACGCCGCTGGCCGGTACTTTAATAGCAAGGTGCCCATCACCAGGACCAAG |
| | AGCACCCCTTTCGAGCTGATCCAGCAGCGCGAGACAAAAGAGGTGGACAGCAA |
| | AGAGAACTTCTCCTACCTGGAAAGCCAGCCTCACGACAGCTGCTTCGTGGAAAT |
| | GCAGGCCCAGAAAGTGATGCACGTGTCCAGCGCCGAGCTGAATTACTCTCTGCC |
| | CTACGACAGCAAGCACCAGATCCGGAACGCCAGCAACGTGAAGCACCACGATA |
| | GCTCTGCCCTGGGAGTGTACAGCTACATTCCCCTGGTGGAAAACCCCTACTTCA |
| | GCTCCTGGCCACCTAGCGGCACAAGCAGCAAGATGTCTCTGGATCTGCCCGAGA |
| | AGCAGGACGGCACAGTGTTCCCATCTAGCCTGCTGCCTACCAGCAGCACCAGCC |
| | TGTTCAGCTACTACAATAGCCACGACTCTCTGTCCCTGAACAGCCCCACCAACA |
| | TCTCCAGCCTGCTGAATCAAGAAAGCGCTGTGCTGGCCACCGCTCCAAGAATCG |
| | ACGATGAGATCCCTCCTCCTCTGCCTGTGCGGACCCCTGAGTCTTTCATCGTGGT |
| | GGAAGAGGCCGGCGAGTTCAGCCCTAATGTGCCCAAATCTCTGAGCAGCGCCGT |
| | GAAAGTCAAGATCGGCACCTCTCTGGAATGGGGCGGCACATCCGAGCCTAAGA |
| | AATTCGACGACTCCGTGATCCTGAGGCCAAGCAAGAGCGTGAAGCTGAGAAGC |
| | CCCAAGTCCGAGCTGCATCAGGACAGATCTAGCCCTCCTCCACCACTGCCTGAG |
| | AGAACCCTCGAGTCATTCTTCCTGGCCGACGAGGATTGCATGCAGGCACAGAGC |
| | ATCGAGACATACAGCACAAGCTACCCCGACACCATGGAAAACAGCACCTCCAG |
| | CAAGCAGACACTGAAAACCCCAGGCAAGAGCTTCACCCGGTCCAAGAGCCTGA |
| | AGATCCTGCGGAACATGAAGAAGTCCATCTGCAACAGCTGCCCTCCAAACAAGC |
| | CTGCCGAGAGCGTGCAGTCCAACAATAGCAGCAGCTTCCTGAACTTCGGCTTTG |
| | CCAACCGGTTCAGCAAGCCTAAGGGCCCCAGAAATCCTCCTCCTACATGGAACA |
| | TC (SEQ ID NO: 24) |
| Zap70 SH2 1, | ATGCCTGATCCTGCTGCTCATCTGCCATTCTTCTACGGCAGCATCAGCAGAGCCG |
| SH2 2 domains | AGGCCGAAGAACATCTGAAGCTGGCCGGAATGGCCGACGGACTGTTTCTGCTCA |
| | GACAGTGCCTGAGAAGCCTCGGCGGCTATGTGCTGTCTCTGGTGCACGATGTGC |
| | GGTTCCATCACTTCCCCATCGAGAGACAGCTGAACGGCACCTACGCTATCGCTG |
| | GCGGAAAAGCCCATTGTGGACCTGCCGAGCTGTGCGAGTTCTACAGCAGAGATC |
| | CTGACGGCCTGCCTTGCAACCTGCGGAAGCCTTGCAATAGACCCAGCGGCCTGG |
| | AACCTCAGCCTGGCGTTTTCGACTGCCTGAGAGATGCCATGGTCCGAGATTACG |
| | TGCGGCAGACCTGGAAGCTGGAAGGCGAAGCTCTGGAACAGGCAATCATCAGC |
| | CAGGCTCCTCAGGTGGAAAAGCTGATCGCCACAACAGCCCACGAGCGGATGCC |
| | TTGGTATCACAGCTCCCTGACCAGAGAGGAAGCCGAGCGGAAGCTGTATTCTGG |
| | CGCCCAGACCGATGGCAAGTTCCTGCTGAGGCCCAGAAAAGAGCAGGGCACAT |
| | ACGCCCTGAGCCTGATCTACGGCAAGACCGTGTACCACTACCTGATCTCCCAGG |
| | ACAAGGCCGGCAAGTACTGTATCCCTGAGGGCACCAAGTTCGACACCCTGTGGC |
| | AGCTGGTGGAATACCTGAAGCTGAAAGCCGATGGACTGATCTACTGCCTGAAAG |
| | AGGCCTGC (SEQ ID NO: 25) |
| Zap70 delta | ATGCCTGATCCTGCTGCTCATCTGCCATTCTTCTACGGCAGCATCAGCAGAGCCG |
| kinase | AGGCCGAAGAACATCTGAAGCTGGCCGGAATGGCCGACGGACTGTTTCTGCTCA |
| | GACAGTGCCTGAGAAGCCTCGGCGGCTATGTGCTGTCTCTGGTGCACGATGTGC |
| | GGTTCCATCACTTCCCCATCGAGAGACAGCTGAACGGCACCTACGCTATCGCTG |
| | GCGGAAAAGCCCATTGTGGACCTGCCGAGCTGTGCGAGTTCTACAGCAGAGATC |
| | CTGACGGCCTGCCTTGCAACCTGCGGAAGCCTTGCAATAGACCCAGCGGCCTGG |
| | AACCTCAGCCTGGCGTTTTCGACTGCCTGAGAGATGCCATGGTCCGAGATTACG |
| | TGCGGCAGACCTGGAAGCTGGAAGGCGAAGCTCTGGAACAGGCAATCATCAGC |
| | CAGGCTCCTCAGGTGGAAAAGCTGATCGCCACAACAGCCCACGAGCGGATGCC |
| | TTGGTATCACAGCTCCCTGACCAGAGAGGAAGCCGAGCGGAAGCTGTATTCTGG |
| | CGCCCAGACCGATGGCAAGTTCCTGCTGAGGCCCAGAAAAGAGCAGGGCACAT |
| | ACGCCCTGAGCCTGATCTACGGCAAGACCGTGTACCACTACCTGATCTCCCAGG |
| | ACAAGGCCGGCAAGTACTGTATCCCTGAGGGCACCAAGTTCGACACCCTGTGGC |
| | AGCTGGTGGAATACCTGAAGCTGAAAGCCGATGGACTGATCTACTGCCTGAAAG |
| | AGGCCTGTCCTAACAGCAGCGCCAGCAATGCTAGCGGAGCTGCTGCACCTACAC |
| | TGCCTGCTCACCCTAGCACACTGACACACCCTCAGCGGAGAATCGATACCCTGA |
| | ACAGCGACGGCTACACCCCAGAACCTGCCAGAATCACTAGCCCCGACAAGCCC |
| | AGACCTATGCCTATGGACACCTCCGTGTACGAGAGCCCCTACAGCGATCCCGAG |
| | GAACTGAAGGACAAGAAGCTGTTCCTCAAGCGGGACAACCTG (SEQ ID |
| | NO: 26) |
| Zap70 Y492F | ATGCCTGATCCTGCTGCTCATCTGCCATTCTTCTACGGCAGCATCAGCAGAGCCG |
| Y493F | AGGCCGAAGAACATCTGAAGCTGGCCGGAATGGCCGACGGACTGTTTCTGCTCA |
| | GACAGTGCCTGAGAAGCCTCGGCGGCTATGTGCTGTCTCTGGTGCACGATGTGC |
| | GGTTCCATCACTTCCCCATCGAGAGACAGCTGAACGGCACCTACGCTATCGCTG |
| | GCGGAAAAGCCCATTGTGGACCTGCCGAGCTGTGCGAGTTCTACAGCAGAGATC |
| | CTGACGGCCTGCCTTGCAACCTGCGGAAGCCTTGCAATAGACCCAGCGGCCTGG |
| | AACCTCAGCCTGGCGTTTTCGACTGCCTGAGAGATGCCATGGTCCGAGATTACG |
| | TGCGGCAGACCTGGAAGCTGGAAGGCGAAGCTCTGGAACAGGCAATCATCAGC |
| | CAGGCTCCTCAGGTGGAAAAGCTGATCGCCACAACAGCCCACGAGCGGATGCC |
| | TTGGTATCACAGCTCCCTGACCAGAGAGGAAGCCGAGCGGAAGCTGTATTCTGG |
| | CGCCCAGACCGATGGCAAGTTCCTGCTGAGGCCCAGAAAAGAGCAGGGCACAT |
| | ACGCCCTGAGCCTGATCTACGGCAAGACCGTGTACCACTACCTGATCTCCCAGG |
| | ACAAGGCCGGCAAGTACTGTATCCCTGAGGGCACCAAGTTCGACACCCTGTGGC |
| | AGCTGGTGGAATACCTGAAGCTGAAAGCCGATGGACTGATCTACTGCCTGAAAG |
| | AGGCCTGTCCTAACAGCAGCGCCAGCAATGCTAGCGGAGCTGCTGCACCTACAC |
| | TGCCTGCTCACCCTAGCACACTGACACACCCTCAGCGGAGAATCGATACCCTGA |
| | ACAGCGACGGCTACACCCCAGAACCTGCCAGAATCACTAGCCCCGACAAGCCC |
| | AGACCTATGCCTATGGACACCTCCGTGTACGAGAGCCCCTACAGCGATCCCGAG |
| | GAACTGAAGGACAAGAAGCTGTTCCTCAAGCGGGACAACCTGCTGATTGCCGA |
| | CATCGAACTCGGCTGCGGCAATTTTGGATCTGTGCGGCAGGGCGTGTACCGGAT |
| | GCGGAAGAAACAGATCGACGTGGCCATCAAGGTGCTGAAGCAGGGAACCGAGA |
| | AGGCCGACACCGAGGAAATGATGCGGGAAGCCCAGATTATGCACCAGCTGGAC |
| | AACCCCTACATCGTGCGGCTGATCGGAGTGTGTCAAGCCGAGGCTCTGATGCTG |
| | GTCATGGAAATGGCAGGCGGAGGCCCTCTGCACAAGTTTCTCGTTGGCAAGCGG |
| | GAAGAGATCCCCGTGTCTAATGTGGCCGAGCTGCTCCACCAAGTGTCTATGGGC |
| | ATGAAGTACCTGGAAGAGAAGAACTTCGTGCACCGCGACCTGGCCGCCAGAAA |
| | TGTGCTGCTGGTCAACAGACACTACGCCAAGATCAGCGACTTCGGCCTGTCTAA |
| | AGCCCTGGGCGCCGACGATAGCTTCTTCACAGCTAGAAGCGCCGGAAAGTGGCC |
| | CCTGAAGTGGTACGCCCCTGAGTGCATCAACTTCCGCAAGTTCAGCTCCAGATC |
| | CGACGTGTGGTCTTACGGCGTGACCATGTGGGAAGCCCTGAGCTACGGCCAGAA |
| | ACCTTACAAGAAGATGAAGGGCCCCGAAGTCATGGCCTTCATCGAACAGGGCA |
| | AGAGAATGGAATGCCCTCCTGAGTGCCCTCCAGAGCTGTATGCCCTGATGAGCG |
| | ATTGCTGGATCTACAAGTGGGAAGATCGGCCCGACTTCCTGACCGTGGAACAGA |
| | GAATGCGGGCCTGCTACTACTCCCTGGCCTCCAAAGTTGAGGGACCTCCTGGCA |
| | GCACACAGAAAGCCGAAGCTGCTTGTGCT (SEQ ID NO: 27) |
| SHIP-1 PTP | GATATGATCACCATCTTCATCGGCACCTGGAATATGGGCAACGCCCCTCCACCT |
| domain | AAGAAGATCACAAGCTGGTTTCTGAGCAAAGGCCAGGGAAAGACCAGGGACGA |
| | CAGCGCCGATTACATCCCTCACGATATCTACGTGATCGGGACCCAAGAGGACCC |
| | TCTGAGCGAGAAAGAGTGGCTGGAAATTCTGAAGCACTCCCTGCAAGAGATCA |
| | CCTCCGTGACCTTCAAGACCGTGGCCATCCACACACTGTGGAACATCCGGATCG |
| | TGGTGCTGGCCAAGCCTGAGCACGAGAACAGAATCAGCCACATCTGCACCGAC |
| | AACGTGAAAACCGGAATCGCCAACACACTGGGCAACAAAGGCGCCGTGGGAGT |
| | GTCCTTCATGTTCAACGGCACCAGCCTGGGCTTCGTGAACAGCCACCTGACATC |
| | CGGCTCCGAGAAGAAGCTGCGGCGGAACCAGAACTATATGAACATCCTGCGGT |
| | TTCTGGCCCTGGGCGACAAGAAGCTGAGCCCCTTCAACATCACCCACCGGTTCA |
| | CCCACCTGTTTTGGTTCGGCGACCTGAACTACAGAGTGGACCTGCCTACCTGGG |
| | AAGCCGAGACAATCATCCAGAAGATCAAGCAGCAACAGTACGCCGACCTGCTG |
| | TCCCACGATCAGCTGCTGACCGAGAGAAGGGAACAGAAAGTGTTCCTGCACTTC |
| | GAGGAAGAGGAAATCACATTCGCCCCTACCTACAGATTCGAGCGGCTGACCCG |
| | GGATAAGTACGCCTACACCAAGCAGAAAGCCACCGGCATGAAGTACAACCTGC |
| | CTTCTTGGTGCGACCGGGTGCTGTGGAAGTCTTACCCTCTGGTGCACGTCGTGTG |
| | CCAGTCTTACGGCAGCACCAGCGACATCATGACCAGCGATCACAGCCCTGTGTT |
| | CGCCACATTTGAGGCCGGCGTGACCAGCCAGTTTGTGTCC (SEQ ID NO: 28) |

### Extracellular Ligand Binding Domain

As used herein, the term “extracellular ligand binding domain” refers to a domain of a chimeric inhibitory protein of the present disclosure that binds to a specific extracellular ligand. Examples of ligand binding domains are known to those having skill in the art and include, but are not limited to, single-chain variable fragments (scFv), natural receptor/ligand domains, and orthogonal dimerization domains such as leucine zippers that engage with a soluble targeting molecule.

In some embodiments, the extracellular ligand binding domain comprises an antigen-binding domain. Antigen-binding domains of the present disclosure can include any domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (VHH) of camelid derived nanobody, and to an alternative scaffold known in the art to function as an antigen-binding domain, such as a recombinant fibronectin domain, a T cell receptor (TCR), a recombinant TCR with enhanced affinity, or a fragment thereof, e.g., single chain TCR, and the like.

In some embodiments, the extracellular ligand binding domain comprises an antibody, or antigen-binding fragment thereof. In some embodiments, the extracellular ligand binding domain comprises a F(ab) fragment, a F(ab′) fragment, a single chain variable fragment (scFv), or a single-domain antibody (sdAb).

The term “single-chain” refers to a molecule comprising amino acid monomers linearly linked by peptide bonds. In certain embodiments, the amino acid monomers are linearly linked by peptide linkers, including, but not limited to, comprises any of the amino acid sequences shown in Table 2. In some embodiments, the peptide linker comprises an amino acid sequence selected from the group consisting of GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), GSTSGSGKPGSGEGSTKG (SEQ ID NO: 44), and EAAAKEAAAKEAAAKEAAAK (SEQ ID NO: 45).

TABLE 2
Peptide Linkers

| Linker | Amino Acid Sequence | SEQ ID NO: |
|---|---|---|
| (G₂S)₁ linker | GGS | 29 |
| (G₂S)₂ linker | GGSGGS | 30 |
| (G₂S)₃ linker | GGSGGSGGS | 31 |
| (G₂S)₄ linker | GGSGGSGGSGGS | 32 |
| (G₂S)₅ linker | GGSGGSGGSGGSGGS | 33 |
| (G₃S)₁ linker | GGGS | 34 |
| (G₃S)₂ linker | GGGSGGGS | 35 |
| (G₃S)₃ linker | GGGSGGGSGGGS | 36 |
| (G₃S)₄ linker | GGGSGGGSGGGSGGGS | 37 |
| (G₃S)₅ linker | GGGSGGGSGGGSGGGSGGGS | 38 |
| (G₄S)₁ linker | GGGGS | 39 |
| (G₄S)₂ linker | GGGGSGGGGS | 40 |
| (G₄S)₃ linker | GGGGSGGGGSGGGGS | 41 |
| (G₄S)₄ linker | GGGGSGGGGSGGGGSGGGGS | 42 |
| (G₄S)₅ linker | GGGGSGGGGSGGGGSGGGGSGGGGS | 43 |
| Whitlow linker | GSTSGSGKPGSGEGSTKG | 44 |
| linker 2 | EAAAKEAAAKEAAAKEAAAK | 45 |

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. As described in more detail herein, an scFv has a variable domain of light chain (VL) connected from its C-terminus to the N-terminal end of a variable domain of heavy chain (VH) by a polypeptide chain. Alternatively, the scFv comprises of polypeptide chain where in the C-terminal end of the VH is connected to the N-terminal end of VL by a polypeptide chain. In certain embodiments, the VH and VL are separated by a peptide linker. In certain embodiments, the scFv peptide linker comprises any of the amino acid sequences shown in Table 2. In certain embodiments, the scFv comprises the structure VH-L-VL or VL-L-VH, wherein VH is the heavy chain variable domain, L is the peptide linker, and VL is the light chain variable domain. In some embodiments, each of the one or more scFvs comprises the structure VH-L-VL or VL-L-VH, wherein VH is the heavy chain variable domain, L is the peptide linker, and VL is the light chain variable domain. When there are two or more scFv linked together, each scFv can be linked to the next scFv with a peptide linked. In some embodiments, each of the one or more scFvs is separated by a peptide linker.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region. In a particular such embodiment, the C-terminus of the Fab light chain is connected to the N-terminus of the Fab heavy chain in a single-chain Fab molecule.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with ß-mercaptoethanol.

“Fv” fragments comprise a non-covalently-linked dimer of one heavy chain variable domain and one light chain variable domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77(1): 13-22).

An “antibody fragment” comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

In some embodiments, the extracellular ligand binding domain comprises a domain from a receptor, wherein the receptor is selected from the group consisting of TCR, BCR, a cytokine receptor, RTK receptors, serine/threonine kinase receptors, hormone receptors, immunoglobulin superfamily receptors, and TNFR-superfamily of receptors

In some embodiments, the extracellular ligand binding domain further comprises a dimerization domain. In some embodiments, the ligand binding domain further comprises a cognate dimerization domain.

As used herein, the term “ligand” refers to a molecule that binds to a site on a cognate protein (i.e., a cognate protein's ligand binding domain), such as a receptor, thereby producing a cellular response/signal, cell-to-cell recognition, and/or cell-to-cell interaction. A ligand may be, for example, one or more diatomic atom (e.g., NO, CO, etc.), small molecule (e.g., a drug, pharmaceutical, simple sugars, nucleotides, nucleotide derivatives, amino acids, amino acid derivatives, small molecule hormones, small-molecule neurotransmitters, etc.), and/or macromolecule (e.g., lipids, polysaccharides, peptides, soluble proteins, cell surface proteins, cytokines, chemokines, hormones, enzymes, etc.). In some embodiments, the ligand is a naturally-occurring biological ligand (i.e., the ligand arises naturally, such as being natively produced by a cell). In other embodiments, the ligand is a non-naturally-occurring or synthetic ligand (i.e., the ligand is produced synthetically such as by chemical synthesis or is engineered to be different in some aspect than a natural ligand, such engineered for expression in a cell that does not typically express the ligand). In some embodiments, a chimeric inhibitory protein can only be activated through binding of a non-naturally-occurring or synthetic ligand. Examples of synthetic ligands include, but are not limited to, drugs, pharmaceuticals, and engineered macromolecules (e.g., synthetic proteins).

In some embodiments, the extracellular ligand binding domain of a chimeric receptor binds to a ligand selected from a protein complex, a protein, a peptide, a receptor-binding domain, a nucleic acid, a small molecule, and a chemical agent. In some embodiments, the ligand is a cytokine, chemokine, hormone, or enzyme.

In some embodiments, the ligand is a cell surface ligand. For example, the ligand of a chimeric inhibitory receptor is present or expressed on a non-target cell surface. Cell surface ligands include, but are not limited to, cell surface markers such as cellular differentiation (CD) markers, receptors, proteins, protein complexes, cell membrane components (e.g., integral membrane proteins, cytoskeletal structures, polysaccharides, lipids, and combinations thereof), and molecules that bind to membrane-associated structures (e.g., soluble antibodies that bind to one or more cell surface ligands). In some embodiments, the cell surface ligand is expressed on a cell that further expresses a cognate ligand of the immune receptor. In some embodiments, the ligand of a chimeric inhibitory receptor is a tumor-associated antigen. In some embodiments, the ligand of a chimeric inhibitory receptor is not expressed on a tumor cell. In some embodiments, the ligand of a chimeric inhibitory receptor is expressed on a non-tumor cell. In some embodiments the ligand of a chimeric inhibitory receptor is expressed on cells of a healthy, or generally considered to be healthy, tissue.

In an illustrative example, chimeric inhibitory receptors are useful as NOT-logic gates for controlling cell activity, such as immune cell activity. Combinations of activating chimeric receptors and chimeric inhibitory receptors, such as those described herein, can be used in the same cell to reduce on-target off-target toxicity. For instance, if a non-target cell expresses both a ligand that is recognized by an activating chimeric receptor and a ligand that is recognized by a chimeric inhibitory receptor, an engineered cell expressing the activating chimeric receptor may bind to the non-target cell and lead to off-target signaling responses. However, in such a case, the same engineered cell also expresses the chimeric inhibitory receptor that can bind its cognate ligand on the non-target cell and the inhibitory function of the chimeric inhibitory receptor can reduce, decrease, prevent, or inhibit signaling meditated by the activating chimeric receptor (“NOT-logic gating”).

In some embodiments, chimeric inhibitory receptors of the present disclosure specifically bind to one or more ligands that are expressed on normal cells (e.g., cells generally considered healthy) but not on tumor cells. In an illustrative non-limiting examples, combinations of tumor-targeting activating chimeric receptors and chimeric inhibitory receptors can be used in the same immunoresponsive cell to reduce on-target off-tumor toxicity. For instance, if a healthy cell expresses both a tumor-associated antigen that is recognized by the tumor-targeting chimeric receptor and an antigen associated with a healthy cell that is recognized by a chimeric inhibitory receptor, an engineered immunoresponsive cell expressing the tumor-targeting chimeric receptor(s) may bind to the healthy cell and lead to off-tumor cellular responses. In such a case, the same engineered immunoresponsive cell also expresses the inhibitory chimeric antigen that can bind its cognate ligand on the healthy cell and the inhibitory function of the chimeric inhibitory receptor can reduce, decrease, prevent, or inhibit the activation of the immunoresponsive cell meditated by the tumor-targeting chimeric receptor.

As used herein, the term “immune receptor” refers to a receptor that binds to a ligand and causes an immune system response. Binding to a ligand in general causes activation of the immune receptor. T cell activation is an example of immune receptor activation. Examples of immune receptors are known to those having skill in the art and include, but are not limited to, T cell receptors, pattern recognition receptors (PRRs; such as NOD-like receptors (NLRs) and Toll-like receptors (TLRs)), killer activated receptors (KARs), killer inhibitor receptors (KIRs), complement receptors, Fc receptors, B cell receptors, NK cell receptors, and cytokine receptors.

Membrane Localization Domain

The chimeric inhibitory receptors include a membrane localization domain. As used herein, the term “membrane localization domain” refers to a region of a chimeric inhibitory receptor of the present disclosure that localizes the receptor to the cell membrane and includes at least a transmembrane domain. In some embodiments, the membrane localization domain of a chimeric receptor further comprises at least a portion of an extracellular domain. In some embodiments, the membrane localization domain further comprises at least a portion of an intracellular domain. In some embodiments, the membrane localization domain further comprises at least a portion of an extracellular domain and at least a portion of an intracellular domain. In some embodiments, the membrane localization domain includes a portion of an extracellular domain, transmembrane domain, and/or intracellular domain that is sufficient to direct or segregate the chimeric inhibitory receptor to a particular domain of the membrane, such as a lipid raft or a heavy lipid raft. In some embodiments, the extracellular ligand binding domain of a chimeric inhibitory receptor is linked to the membrane localization domain through an extracellular linker region, such as the peptide linkers shown in Table 2.

In some embodiments, the membrane localization domain comprises a transmembrane domain selected from an LAX transmembrane domain, a CD25 transmembrane domain, a CD7 transmembrane domain, a LAT transmembrane domain, a transmembrane domain from a LAT mutant (see e.g., Pavel Otáhal et al., Biochim Biophys Acta. 2011 February; 1813(2):367-76), a BTLA transmembrane domain, a CD8 transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAGS transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.

In some embodiments, the transmembrane domain is derived from a CD8 polypeptide. Any suitable CD8 polypeptide may be used. Exemplary CD8 polypeptides include, without limitation, NCBI Reference Nos. NP_001139345 and AAA92533.1. In some embodiments, the transmembrane domain is derived from a CD28 polypeptide. Any suitable CD28 polypeptide may be used. Exemplary CD28 polypeptides include, without limitation, NCBI Reference Nos. NP_006130.1 and NP_031668.3. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1. In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide. Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. Any suitable LIR-1 (LILRB1) polypeptide may be used. Exemplary LIR-1 (LILRB1) polypeptides include, without limitation, NCBI Reference Nos. NP_001075106.2 and NP_001075107.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2, or fragments thereof. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NG2C.

In some embodiments, the transmembrane domain derived from a LAT mutant is derived from a LAT(CA) mutant. See e.g., Kosugi A., et al. Involvement of SHP-1 tyrosine phosphatase in TCR-mediated signaling pathways in lipid rafts, Immunity, 2001 June; 14(6): 669-80.

In some embodiments, the transmembrane domain is selected from the amino acid sequences shown in Table 3. In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to any of the sequences shown in Table 3. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the transmembrane domain is a nucleic acid sequence that is at least 60%, at least 65%, at least 70%, at least 75%, at least 80%, or at least 85% identical to any of the nucleic acid sequences listed in Table 3. In some embodiments, the transmembrane domain is a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 3.

TABLE 3
Transmembrane Domains

| TM domain | Amino Acid Sequence | DNA Sequence |
|---|---|---|
| CD28 | FWVLVVVGGVLACYSLLVT | TTTTGGGTGCTGGTGGTGGTTGGTGGAGTCCTGGCTT |
| | VAFIIFWV (SEQ ID NO: | GCTATAGCTTGCTAGTAACAGTGGCCTTTATTATTTT |
| | 65) | CTGGGTG (SEQ ID NO: 73) |
| PAG | GPAGSLLGSGQMQITLWGS | GGTCCGGCTGGCTCTCTGCTCGGCAGTGGTCAGATG |
| | LAAVAIFFVITFLIFLCSSCD | CAGATTACGTTGTGGGGCAGTTTGGCAGCCGTCGCA |
| | REKKPR (SEQ ID NO: 66) | ATCTTCTTTGTTATCACTTTTCTTATCTTTCTCTGTTC |
| | | CTCATGTGACAGAGAGAAAAAGCCCCGA (SEQ ID |
| | | NO: 74) |
| mLAT | MEADALSPVGLGLLLLPFL | ATGGAAGCCGATGCTCTGTCTCCTGTTGGCCTGGGA |
| | VTLLAALCVRCRELPVS | CTGCTGCTCCTGCCTTTTCTGGTTACACTGCTGGCCG |
| | (SEQ ID NO: 67) | CTCTGTGTGTGCGGTGTAGAGAACTGCCAGTTAGT |
| | | (SEQ ID NO: 75) |
| mLAT(ca) | MEADALSPVGLGLLLLPFL | ATGGAAGCCGATGCTCTGTCTCCTGTTGGCCTGGGA |
| | VTLLAALAVRARELPVS | CTGCTGCTCCTGCCTTTTCTCGTTACACTGCTGGCCG |
| | (SEQ ID NO: 68) | CTCTGGCTGTGCGAGCTAGAGAACTGCCTGTGTCT |
| | | (SEQ ID NO: 76) |
| LAT (1-33) | EEAILVPCVLGLLLLPILAM | GAGGAAGCAATCCTGGTGCCGTGTGTACTTGGTCTG |
| | LMALCVHCHRLP (SEQ ID | CTTTTGTTGCCAATACTTGCGATGCTCATGGCTCTCT |
| | NO: 69) | GCGTACATTGCCATCGGCTTCCG (SEQ ID NO: 77) |
| LAX | IFSGFAGLLAILLVVAVFCIL | ATCTTCAGCGGCTTTGCCGGACTGCTGGCTATCCTGC |
| | (SEQ ID NO: 70) | TGGTTGTGGCCGTGTTCTGTATCCTT (SEQ ID NO: |
| | | 78) |
| CD3z | LCYLLDGILFIYGVILTALFL | CTGTGCTACCTGCTGGACGGCATCCTGTTTATCTACG |
| | (SEQ ID NO: 71) | GCGTGATCCTGACAGCCCTGTTCCTT (SEQ ID NO: |
| | | 79) |
| CD45 | ALIAFLAFLIIVTSIALLVVL | GCCCTGATTGCCTTCCTGGCCTTTCTGATCATCGTGA |
| | (SEQ ID NO: 72) | CCAGCATTGCCCTGCTGGTCGTGCTG (SEQ ID NO: |
| | | 80) |

In some embodiments, the membrane localization domain further comprises at least a portion of a corresponding extracellular domain and/or at least a portion of a corresponding intracellular domain (see, e.g., spacers and hinges described herein derived from the membrane localization domains described herein).

In some embodiments, the membrane localization domain further comprises proximal protein fragments. Proximal protein fragments refer to protein segments immediately adjacent to transmembrane domains in their native context. For example, proximal protein fragments can be protein segments that fall outside a transmembrane domain of a protein or that fall outside the conventional boundary of a sequence considered to be a transmembrane domain of a protein. In some embodiments, proximal protein fragments can be a spacer or hinge sequence. In some embodiments, proximal protein fragments can be distinct from a spacer or hinge sequence.

In some embodiments, the membrane localization domain directs or segregates the chimeric inhibitory receptor to a domain of a cell membrane. As used herein, the term “domain of a cell membrane” refers to a lateral inhomogeneity in lipid composition and physical properties in a cell membrane. Cell membrane domain formation may be driven by multiple forces: hydrogen bonding, hydrophobic entropic forces, charge pairing and van der Waals forces. Cell membrane domains may arise via protein-protein interactions within membranes, protein-lipid interactions within membranes, or lipid-lipid interactions within membranes. Examples of cell membrane domains are known to those having skill in the art and include, but are not limited to, lipid rafts, heavy lipid rafts, light lipid rafts, caveolae, patches, posts, fences, lattices, rafts, and scaffolds. See e.g., Nicolson G. L., The Fluid-Mosaic Model of Membrane Structure: still relevant to understanding the structure, function and dynamics of biological membranes after more than 40 years, Biochim. Biophys. Acta. 2014 June; 1838(6): 1451-66.

In some embodiments, the membrane localization domain localizes a chimeric inhibitory receptor of the present disclosure to a lipid raft. In some embodiments, the membrane localization domain interacts with one or more cell membrane components localized in a domain of a cell membrane. Examples of cell membrane components are known to those having skill in the art and include, but are not limited to, various integral membrane proteins, cytoskeletal structures, polysaccharides, lipids, and combinations thereof. See e.g., Nicolson G. L., The Fluid-Mosaic Model of Membrane Structure: still relevant to understanding the structure, function and dynamics of biological membranes after more than 40 years, Biochim. Biophys. Acta. 2014 June; 1838(6): 1451-66.

In some embodiments, the membrane localization domain mediates basal localization (i.e., localization in the absence of cognate ligand) of the chimeric inhibitory receptor to a domain of a cell membrane that is distinct from domains of the cell membrane occupied by one or more components of an immune receptor, such as a membrane portion distinct from a lipid raft occupied by an immune receptor. In some embodiments, the basal membrane localization domain is sufficient to mitigate constitutive inhibition of immune receptor activation by the enzymatic inhibitory domain.

As used herein, the term “immune receptor activation” refers to an event that initiates a signaling cascade that ultimately results in an immune response. T cell activation is an example of immune receptor activation. In general, and without wishing to be bound by theory, while a membrane localization domain can mitigate constitutive inhibition of an immune receptor, binding between the chimeric inhibitory receptor and its cognate ligand generally mediates spatial recruitment of the enzymatic inhibitory domain to be proximal to the immune receptor and/or downstream signaling complexes such that the enzymatic inhibitory domain is capable of negatively regulating an intracellular signal transduction cascade. In a non-limiting illustrative example, binding between the chimeric inhibitory receptor and its cognate ligand can localize the receptor and enzymatic inhibitory domain to an immunological synapse and inhibit immune receptor signaling and/or activation, such as T cell activation (e.g., a inhibit a TCR present in the immunological synapse, such an TCRs bound to its cognate ligand), either directly acting on the immune receptor and/or on another signaling component involved in an intracellular signal transduction cascade.

In some embodiments, a non-specific transmembrane domain will be sufficient to prevent the enzymatic inhibitory domain from constitutively inhibiting T cell activation. In other embodiments, a transmembrane domain (including proximal protein fragments) can be selected that mediates localization to regions of the cell membrane that are physically distinct from those regions occupied by components of the T-cell receptor (e.g., segregation to “heavy” lipid rafts, instead of “classical” lipid rafts; see e.g., Stanford et al., Regulation of TCR signaling by tyrosine phosphatases: from immune homeostasis to autoimmunity, Immunology, 2012 September; 137(1): 1-19), such as regions of the cell membrane other than an immunological synapse.

### Spacers and Hinge Domains

Chimeric inhibitory receptors can also contain spacer or hinge domains. In some embodiments, a spacer domain or a hinge domain is located between an extracellular domain (e.g., comprising the extracellular ligand binding domain) and a transmembrane domain of an chimeric inhibitory receptor, or between an intracellular signaling domain and a transmembrane domain of the chimeric inhibitory receptor. A spacer or hinge domain is any oligopeptide or polypeptide that functions to link the transmembrane domain to the extracellular domain and/or the intracellular signaling domain in the polypeptide chain. Spacer or hinge domains can provide flexibility to the chimeric inhibitory receptor, or domains thereof, or prevent steric hindrance of the chimeric inhibitory receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an chimeric inhibitory receptor. In some embodiments, a spacer or hinge domain includes at least a portion of an extracellular domain and/or at least a portion of an intracellular domain from the same source as the membrane localization domain.

Exemplary spacer or hinge domain protein sequences are shown in Table 4. Exemplary spacer or hinge domain nucleotide sequences are shown in Table 5. In some embodiments, a spacer or hinge domain is an amino acid sequence that is at least 90%, at least 91%, at least 92%, at least 93%, at least 94%, at least 95%, at least 96%, at least 97%, at least 98%, or at least 99% identical to any of the amino acid sequences listed in Table 4. In some embodiments, a spacer or hinge domain is a nucleic acid sequence that is at least 60%, at least 65%, at least 70%, at least 75%, at least 80%, or at least 85% identical to any of the nucleic acid sequences listed in Table 5. In some embodiments, a spacer or hinge domain is a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 5.

TABLE 4
Spacer/Hinge Domain Amino Acid Sequences

| | SEQ | |
|---|---|---|
| Amino Acid Sequence | ID NO: | Description |
| AAAIEVMYPPPYLDNEKSNGTIIHVKGKHLCPSPLFP | 46 | CD28 hinge |
| GPSKP | | |
| ESKYGPPCPSCP | 47 | IgG4 minimal hinge |
| ESKYGPPAPSAP | 48 | IgG4 minimal hinge, no |
| | | disulfides |
| ESKYGPPCPPCP | 49 | IgG4 S228P minimal hinge, |
| | | enhanced disulfide formation |
| EPKSCDKTHTCP | 50 | IgG1 minimal hinge |
| AAAFVPVFLPAKPTTTPAPRPPTPAPTIASQPLSLRPE | 51 | Extended CD8a hinge |
| ACRPAAGGAVHTRGLDFACDIYIWAPLAGTCGVLLL | | |
| SLVITLYCNHRN | | |
| TTTPAPRPPTPAPTIALQPLSLRPEACRPAAGGAVHTR | 52 | CD8a hinge |
| GLDFACD | | |
| ACPTGLYTHSGECCKACNLGEGVAQPCGANQTVCE | 53 | LNGFR hinge |
| PCLDSVTFSDVVSATEPCKPCTECVGLQSMSAPCVEA | | |
| DDAVCRCAYGYYQDETTGRCEACRVCEAGSGLVFS | | |
| CQDKQNTVCEECPDGTYSDEADAEC | | |
| ACPTGLYTHSGECCKACNLGEGVAQPCGANQTVC | 54 | Truncated LNGFR hinge (TNFR- |
| | | Cys1) |
| AVGQDTQEVIVVPHSLPFKV | 55 | PDGFR-beta extracellular linker |

TABLE 5
Spacer/Hinge Domain Nucleic Acid Sequences

| Nucleic Acid Sequence | SEQ ID NO: | Description |
|---|---|---|
| GCAGCAGCTATCGAGGTGATGTATCCTCCGCCCTA | 56 | CD28 hinge |
| CCTGGATAATGAAAAGAGTAATGGGACTATCATTC | | |
| ATGTAAAAGGGAAGCATCTTTGTCCTTCTCCCCTTT | | |
| TCCCCGGTCCGTCTAAACCT | | |
| GAA AGC AAG TAC GGT CCA CCT TGC CCT AGC | 57 | IgG4 minimal hinge |
| TGT CCG | | |
| GAA TCC AAG TAC GGC CCC CCA GCG CCT AGT | 58 | IgG4 minimal hinge, no |
| GCC CCA | | disulfides |
| GAA TCT AAA TAT GGC CCG CCA TGC CCG CCT | 59 | IgG4 S228P minimal hinge, |
| TGC CCA | | enhanced disulfide formation |
| GAA CCG AAG TCT TGT GAT AAA ACT CAT ACG | 60 | IgG1 minimal hinge |
| TGC CCG | | |
| GCT GCT GCT TTC GTA CCC GTG TTC CTC CCT | 61 | Extended CD8a hinge |
| GCT AAG CCT ACG ACT ACC CCC GCA CCG AGA | | |
| CCA CCC ACG CCA GCA CCC ACG ATTGCT AGC | | |
| CAG CCC CTT AGT TTG CGA CCA GAA GCT TGT | | |
| CGG CCT GCT GCT GGT GGC GCG GTA CAT ACC | | |
| CGC GGC CTT GAT TTT GCTTGC GAT ATA TAT | | |
| ATC TGG GCG CCT CTG GCC GGA ACA TGC GGG | | |
| GTC CTC CTC CTT TCT CTG GTT ATT ACT CTC | | |
| TAC TGT AAT CACAGG AAT | | |
| GCC TGC CCG ACC GGG CTC TAC ACT CAT AGC | 62 | LNGFR hinge |
| GGG GAA TGT TGT AAG GCA TGT AAC TTG GGT | | |
| GAG GGC GTC GCA CAG CCC TGC GGAGCT AAC | | |
| CAA ACA GTG TGC GAA CCC TGC CTC GAT AGT | | |
| GTG ACG TTC TCT GAT GTT GTA TCA GCT ACA | | |
| GAG CCT TGC AAA CCA TGTACT GAG TGC GTT | | |
| GGA CTT CAG TCA ATG AGC GCT CCA TGT GTG | | |
| GAG GCA GAT GAT GCG GTC TGT CGA TGT GCT | | |
| TAC GGA TAC TACCAA GAC GAG ACA ACA GGG | | |
| CGG TGC GAG GCC TGT AGA GTT TGT GAG GCG | | |
| GGC TCC GGG CTG GTG TTT TCA TGT CAA GAC | | |
| AAG CAAAAT ACG GTC TGT GAA GAG TGC CCT | | |
| GAT GGC ACC TAC TCA GAC GAA GCA GAT GCA | | |
| GAA TGC | | |
| GCC TGC CCT ACA GGA CTC TAC ACG CAT AGC | 63 | Truncated LNGFR hinge (TNFR- |
| GGT GAG TGT TGT AAA GCA TGC AAC CTC GGG | | Cys1) |
| GAA GGT GTA GCC CAG CCA TGC GGG GCT AAC | | |
| CAA ACC GTT TGC | | |
| GCTGTGGGCCAGGACACGCAGGAGGTCATCGTGG | 64 | PDGFR-beta extracellular |
| TGCCACACTCCTTGCCCTTTAAGGTG | | linker |

In some embodiments, the chimeric inhibitory receptor further comprises a spacer region between the extracellular ligand binding domain and the membrane localization domain, also referred to as an extracellular linker. In some embodiments, the extracellular linker region is positioned between the extracellular ligand binding domain and membrane localization domain and operably and/or physically linked to each of the extracellular ligand binding domain and the membrane localization domain.

In some embodiments, the chimeric inhibitory receptor further comprises a spacer region between the membrane localization domain and the enzymatic inhibitory domain, also referred to as an intracellular spacer region. In some embodiments, the chimeric inhibitory receptor further comprises an intracellular spacer region positioned between the membrane localization domain and the enzymatic inhibitory domain and operably and/or physically linked to each of the membrane localization domain and the enzymatic inhibitory domain.

In some embodiments, the extracellular linker region and/or intracellular spacer region is derived from a protein selected from the group consisting of: CD8α, CD4, CD7, CD28, IgG1, IgG4, FcγRIIIα, LNGFR, and PDGFR. In some embodiments, the extracellular linker region and/or intracellular spacer region comprises an amino acid sequence selected from the group consisting of:

(SEQ ID NO: 46)
AAAIEVMYPPPYLDNEKSNGTIIHVKGKHLCPSPLFPGPSKP,
(SEQ ID NO: 47)
ESKYGPPCPSCP,
(SEQ ID NO: 48)
ESKYGPPAPSAP,
(SEQ ID NO: 49)
ESKYGPPCPPCP,
(SEQ ID NO: 50)
EPKSCDKTHTCP,
(SEQ ID NO: 51)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 52)
TTTPAPRPPTPAPTIALQPLSLRPEACRPAAGGAVHTRGLDFACD.
(SEQ ID NO: 53)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 54)
ACPTGLYTHSGECCKACNLGEGVAQPCGANQTVC,
and
(SEQ ID NO: 55)
AVGQDTQEVIVVPHSLPFKV.

In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:46. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:47. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:48. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:49. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:50. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:51. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:52. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:53. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:54. In some embodiments, the extracellular linker region and/or intracellular spacer region comprises an amino acid sequence that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identical to SEQ ID NO:55.

In some embodiments, the extracellular linker region and/or intracellular spacer region includes a peptide linker, such as any of the amino acid sequences shown in Table 2. In some embodiments, the extracellular linker region and/or intracellular spacer region includes a peptide linker having the amino acid sequence selected from the group consisting of GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), GSTSGSGKPGSGEGSTKG (SEQ ID NO: 44), and EAAAKEAAAKEAAAKEAAAK (SEQ ID NO: 45).

In some embodiments, the extracellular linker region and/or intracellular spacer region modulates sensitivity of the chimeric inhibitory receptor. In some embodiments, the extracellular linker region and/or intracellular spacer region increases sensitivity of the chimeric inhibitory receptor relative to an otherwise identical chimeric inhibitory receptor lacking the extracellular linker region and/or intracellular spacer region. In some embodiments, the extracellular linker region and/or intracellular spacer region reduces sensitivity of the chimeric inhibitory receptor relative to an otherwise identical chimeric inhibitory receptor lacking the extracellular linker region and/or intracellular spacer region. In some embodiments, the extracellular linker region and/or intracellular spacer region modulates potency of the chimeric inhibitory receptor relative to an otherwise identical chimeric inhibitory receptor lacking the extracellular linker region and/or intracellular spacer region. In some embodiments, the extracellular linker region and/or intracellular spacer region increases potency of the chimeric inhibitory receptor relative to an otherwise identical chimeric inhibitory receptor lacking the extracellular linker region and/or intracellular spacer region. In some embodiments, the extracellular linker region and/or intracellular spacer region reduces potency of the chimeric inhibitory receptor relative to an otherwise identical chimeric inhibitory receptor lacking the extracellular linker region and/or intracellular spacer region. In some embodiments, the extracellular linker region and/or intracellular spacer region modulates basal prevention, attenuation, or inhibition of activation of the tumor-targeting chimeric receptor expressed on the engineered cell relative to an otherwise identical chimeric inhibitory receptor lacking the extracellular linker region and/or intracellular spacer region. In some embodiments, the extracellular linker region and/or intracellular spacer region reduces basal prevention, attenuation, or inhibition relative to an otherwise identical chimeric inhibitory receptor lacking the extracellular linker region and/or intracellular spacer region. In some embodiments, the extracellular linker region and/or intracellular spacer region increases basal prevention, attenuation, or inhibition relative to an otherwise identical chimeric inhibitory receptor lacking the extracellular linker region and/or intracellular spacer region.

In some embodiments, the chimeric inhibitory receptor further comprises an intracellular spacer region positioned between the transmembrane domain and the intracellular signaling domain and is operably linked to each of the transmembrane domain and the intracellular signaling domain. In some embodiments, the chimeric inhibitory receptor further comprises an intracellular spacer region positioned between the transmembrane domain and the intracellular signaling domain and is physically linked to each of the transmembrane domain and the intracellular signaling domain.

In some embodiments, the intracellular spacer region modulates sensitivity of the chimeric inhibitory receptor relative to an otherwise identical chimeric inhibitory receptor lacking the intracellular spacer region. In some embodiments, the intracellular spacer region increases sensitivity of the chimeric inhibitory receptor relative to an otherwise identical chimeric inhibitory receptor lacking the intracellular spacer region. In some embodiments, the intracellular spacer region reduces sensitivity of the chimeric inhibitory receptor relative to an otherwise identical chimeric inhibitory receptor lacking the intracellular spacer region. In some embodiments, the intracellular spacer region modulates potency of the chimeric inhibitory receptor relative to an otherwise identical chimeric inhibitory receptor lacking the intracellular spacer region.

In some embodiments, the intracellular spacer region increases potency of the chimeric inhibitory receptor relative to an otherwise identical chimeric inhibitory receptor lacking the intracellular spacer region. In some embodiments, the intracellular spacer region reduces potency of the chimeric inhibitory receptor relative to an otherwise identical chimeric inhibitory receptor lacking the intracellular spacer region. In some embodiments, the intracellular spacer region modulates basal prevention, attenuation, or inhibition of activation of the tumor-targeting chimeric receptor expressed on the engineered cell when expressed on an engineered cell relative to an otherwise identical chimeric inhibitory receptor lacking the intracellular spacer region. In some embodiments, the intracellular spacer region reduces basal prevention, attenuation, or inhibition relative to an otherwise identical chimeric inhibitory receptor lacking the intracellular spacer region. In some embodiments, the intracellular spacer region increases basal prevention, attenuation, or inhibition relative to an otherwise identical chimeric inhibitory receptor lacking the intracellular spacer region.

### Intracellular Inhibitory Co-signaling Domains

In some embodiments, the chimeric inhibitory receptors comprises one or more intracellular inhibitory co-signaling domains. In some embodiments, the one or more intracellular inhibitory co-signaling domains are between the membrane localization domain and the enzymatic inhibitory domain. In some embodiments, the one or more intracellular inhibitory co-signaling domains are between the transmembrane domain and the and the enzymatic inhibitory domain. In some embodiments, the one or more intracellular inhibitory co-signaling domains are C-terminal of the enzymatic inhibitory domain. In some embodiments, the one or more intracellular inhibitory co-signaling domains are linked to other domains (e.g., a membrane localization, a transmembrane domain, or an enzymatic inhibitory domain) through a peptide linker (e.g., see Table 2) or a spacer or hinge sequence (e.g., see Table 4). In some embodiments, when two or more intracellular inhibitory co-signaling domains are present, the two or more intracellular inhibitory co-signaling domains can be linked through a peptide linker (e.g., see Table 2) or a spacer or hinge sequence (e.g., see Table 4).

In some embodiments, the one or more intracellular inhibitory co-signaling domains of a chimeric protein comprises one or more ITIM-containing protein, or fragment(s) thereof. ITIMs are conserved amino acid sequences found in cytoplasmic tails of many inhibitory immune receptors. In some embodiments, the one or more ITIM-containing protein, or fragments thereof, is selected from PD-1, CTLA4, TIGIT, BTLA, and LAIR1. In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAGS, HAVR, GITR, and PD-L1. In some embodiments, the inhibitory mechanisms of the enzymatic inhibitory domain and the ITIM and/or non-ITIM scaffolds overlap, e.g., an ITIM-containing protein recruits the endogenous version of the enzyme from which the enzymatic inhibitory domain is derived, such as SHP-1. In some embodiments, the inhibitory mechanisms of the enzymatic inhibitory domain and the ITIM and/or non-ITIM scaffolds are distinct and can be complementary/synergistic, e.g., the activities of an ITIM-containing protein and a Csk or CBL-b derived enzymatic inhibitory domain.

### Immune Receptors

In some embodiments, the immune receptor is a naturally-occurring immune receptor. In some embodiments, the immune receptor is a naturally-occurring antigen receptor. In some embodiments, the immune receptor is selected from a T cell receptor (TCR), a pattern recognition receptor (PRR), a NOD-like receptor (NLR), a Toll-like receptor (TLR), a killer activated receptor (KAR), a killer inhibitor receptor (KIR), an NK cell receptor, a complement receptor, an Fc receptor, a B cell receptor, and a cytokine receptor. In some embodiments, the immune receptor is a TCR.

In some embodiments, the immune receptor is a chimeric immune receptor. In some embodiments, the immune receptor is a chimeric antigen receptor (CAR). In general, as used herein and unless otherwise specified, immune receptors in a CAR format refer to activating CARs that typically are a recombinant polypeptide construct comprising at least an extracellular antigen-binding domain, a transmembrane domain and a cytoplasmic signaling domain (also referred to herein as “an intracellular signaling domain”) comprising a functional signaling domain derived from a stimulatory molecule as defined below. A CAR of the present disclosure may be a first, second, or third generation CAR. “First generation” CARs comprise a single intracellular signaling domain, generally derived from a T cell receptor chain. “First generation” CARs generally have the intracellular signaling domain from the CD3-zeta (CD3ζ) chain, which is the primary transmitter of signals from endogenous TCRs. “First generation” CARs can provide de novo antigen recognition and cause activation of both CD4+ and CD8+ T cells through their CD3ζ chain signaling domain in a single fusion molecule, independent of HLA-mediated antigen presentation. “Second generation” CARs add a second intracellular signaling domain from one of various co-stimulatory molecules (e.g., CD28, 4-1BB, ICOS, OX40) to the cytoplasmic tail of the CAR to provide additional signals to the T cell. “Second generation” CARs provide both co-stimulation (e.g., CD28 or 4-1BB) and activation (CD3ζ). Preclinical studies have indicated that “Second Generation” CARs can improve the anti-tumor activity of immunoresponsive cell, such as a T cell. “Third generation” CARs have multiple intracellular co-stimulation signaling domains (e.g., CD28 and 4-1BB) and an intracellular activation signaling domain (CD3ζ).

In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. In some embodiments, the stimulatory molecule is the zeta chain associated with the T cell receptor complex. In some embodiments, the cytoplasmic signaling domain comprises a primary signaling domain (e.g., a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 4-1BB (i.e., CD 137), CD27, ICOS, and/or CD28. In some embodiments, the CAR. comprises a chimeric fusion protein comprising an extracellular antigen-binding domain, a transmembrane domain and an intracellular signaling domain comprising a functional signaling domain derived from a stimulatory molecule. In some embodiments, the CAR comprises a chimeric fusion protein comprising an extracellular antigen-binding domain, a transmembrane domain and an intracellular signaling domain comprising a functional signaling domain derived from a co-stimulatory molecule and a functional signaling domain derived from a stimulatory molecule. In some embodiments, the CAR comprises a chimeric fusion protein comprising an extracellular antigen-binding domain, a transmembrane domain and an intracellular signaling domain comprising two functional signaling domains derived from one or more co-stimulatory molecule(s) and a functional signaling domain derived from a stimulatory molecule. In some embodiments, the CAR comprises a chimeric fusion protein comprising an extracellular antigen-binding domain, a transmembrane domain and an intracellular signaling domain comprising at least two functional signaling domains derived from one or more co-stimulatory molecule(s) and a functional signaling domain derived from a stimulatory molecule. In some embodiments, the CAR comprises an optional leader sequence (also referred to as a signal sequence) at the amino-terminus (N-ter) of the CAR fusion protein. In some embodiments, the CAR further comprises a leader sequence at the N-terminus of the extracellular antigen-binding domain, wherein the leader sequence is optionally cleaved from the antigen recognition domain (e.g., an scFv) during cellular processing and localization of the CAR to the cellular membrane.

Various chimeric antigen receptors are known in the art including, but not limited to, ScFv-FcεRIγCAIX, ScFv-FcεRIγ, ScFv-CD3ζ; ScFv-CD28-CD3ζ; ScFv-CD28-CD3ζ, ScFv-CD3ζ; ScFv-CD4-CD3ζ; CD3ζ/CD137/CD28, ScFv-CD28-41BB-CD3ζ; ScFv-CD8-CD3ζ, ScFv-FceRIγ, CD28/4-1BB-CD3ζ, ScFv-CD28mut-CD3ζ, Heregulin-CD3ζ; ScFv-CD28, ScFv-CD28-OX40-CD3ζ, ScFv-CD3ζ, IL-13-CD28-4-1BB-CD3ζ, IL-13-CD3; IL-13-CD3; ScFv-FcεRIγ, ScFV-CD4-FcεRIγ, ScFV-CD28-FcεRIγ, Ly49H-CD3ζ, NKG2D-CD3ζ, ScFv-b2c-CD3ζ, and FceRI-CD28-CD3ζ. In some embodiments, the chimeric antigen receptor has been modified to include control elements. In some embodiments, the chimeric antigen receptor is a split chimeric antigen receptor; see e.g., WO2017/091546.

In some embodiments, the immune receptor is a chimeric TCR. A chimeric TCR generally includes an extracellular ligand binding domain grafted onto one or more constant domains of a TCR chain, for example a TCR alpha chain or TCR beta chain, to create a chimeric TCR that binds specifically to an antigen of interest, such a tumor-associated antigen. Without wishing to be bound by theory, it is believed that chimeric TCRs may signal through the TCR complex upon antigen binding. For example, an antibody or antibody fragment (e.g., scFv) can be grafted to the constant domain (e.g., at least a portion of the extracellular constant domain, the transmembrane domain and cytoplasmic domain) of a TCR chain, such as the TCR alpha chain and/or the TCR beta chain. As another example, the CDRs of an antibody or antibody fragment may be grafted into a TCR alpha chain and/or beta chain to create a chimeric TCR that binds specifically to an antigen. Such chimeric TCRs may be produced by methods known in the art (e.g., Willemsen R A et al., Gene Therapy 2000; 7:1369-1377; Zhang T et al., Cancer Gene Ther 2004 11: 487-496; and Aggen et al., Gene Ther. 2012 April; 19(4): 365-74; herein incorporated by reference for all purposes).

The antigen of an immune receptor, such as a chimeric antigen receptor, can be a tumor-associated antigen.

Immune receptors generally are capable of inducing signal transduction or changes in protein expression in the immune receptor-expressing cell that results in the modulation of an immune response upon binding to a cognate ligand (e.g., regulate, activate, initiate, stimulate, increase, prevent, attenuate, inhibit, reduce, decrease, inhibit, or suppress an immune response). For example, when CD3 chains present in a TCR/CAR cluster in response to ligand binding, an immunoreceptor tyrosine-based activation motifs (ITAMs)-meditated signal transduction cascade is produced. Specifically, in certain embodiments, when an endogenous TCR, exogenous TCR, chimeric TCR, or a CAR (specifically an activating CAR) binds their respective antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated that in turn can initiate a T cell activation pathway and ultimately activates transcription factors, such as NF-κB and AP-1. These transcription factors are capable of inducing global gene expression of the T cell to increase IL-2 production for proliferation and expression of master regulator T cell proteins in order to initiate a T cell mediated immune response, such as cytokine production and/or T cell mediated killing.

### Nucleic Acids Encoding Chimeric Inhibitory Receptors

Provided herein, in other aspects, are nucleic acids encoding at least one chimeric inhibitory receptor as described above. In some embodiments, the nucleic acid encoding the at least one chimeric inhibitory receptor is a vector. In some embodiments, the vector is selected from a plasmid vector, a viral vector, a lentiviral vector, or a phage vector.

When the chimeric inhibitory receptor is a multichain receptor, a set of polynucleotides is used. In this case, the set of polynucleotides can be cloned into a single vector or a plurality of vectors. In some embodiments, the polynucleotide comprises a sequence encoding a chimeric inhibitory receptor, wherein the sequence encoding an extracellular ligand binding domain is contiguous with and in the same reading frame as a sequence encoding an intracellular signaling domain and a membrane localization domain.

The polynucleotide can be codon optimized for expression in a mammalian cell. In some embodiments, the entire sequence of the polynucleotide has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148, herein incorporated by reference for all purposes.

The polynucleotide encoding a chimeric inhibitory receptor can be obtained using recombinant methods known in the art, such as, for example by screening libraries from cells expressing the polynucleotide, by deriving it from a vector known to include the same, or by isolating directly from cells and tissues containing the same, using standard techniques. Alternatively, the polynucleotide can be produced synthetically, rather than cloned.

The polynucleotide can be cloned into a vector. In some embodiments, an expression vector known in the art is used. Accordingly, the present disclosure includes retroviral and lentiviral vector constructs expressing a chimeric inhibitory receptor that can be directly transduced into a cell.

The present disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail. RNA so produced can efficiently transfect different kinds of cells. In some embodiments, an RNA chimeric inhibitory receptor vector is transduced into a cell, e.g., a T cell or a NK cell, by electroporation.

In some embodiments, a vector of the present disclosure may further comprise a signal sequence to facilitate secretion, a polyadenylation signal and transcription terminator, an element allowing episomal replication, and/or elements allowing for selection.

### Engineered Cells

Also provided herein are genetically engineered cells comprising a nucleic acid encoding at least one chimeric inhibitory receptor of the present disclosure or that express a chimeric inhibitory receptor of the present disclosure. Various ways of introducing nucleic acids/vectors (i.e., genetically engineering) are known to those having skill in the art and include, but are not limited to, transduction (i.e., viral infection), transformation, and transfection. Mechanisms of transfection include chemical-based transfection (e.g., calcium phosphate-mediated, lipofection/liposome mediated, etc.), non-chemical-based transfection (e.g., electroporation, cell squeezing, sonoporation, optical transfection, protoplast fusion, impalefection, hydrodynamic delivery, etc.), and particle-based transfection (e.g., gene gun, magnetofection, particle bombardment, etc.).

In some embodiments, a genetically engineered cell of the present disclosure is an immunomodulatory cell. Immunomodulatory cells include, but are not limited to, a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

In some embodiments, a genetically engineered cell of the present disclosure is an immune cell. In some embodiments, the immune cell is a T cell. Examples of T cells include, but are not limited to CD8+ T cells, CD4+ T cells, effector cells, helper cells (T_{H} cells), cytotoxic cells (T_{C} cells, CTLs, T-killer cells, killer T cells), memory cells (central memory T cells, effector memory T cells, tissue resident memory T cells, virtual memory T cells, etc.), regulatory T cells (e.g., CD4+, FOXP3+, CD25+), natural killer T cells, mucosal associated invariant cells, and gamma delta T cells. In some embodiments, the immune cell is a

In some embodiments, a genetically engineered cell of the present disclosure is a stem cell, such as a mesenchymal stem cell (MSC), pluripotent stem cell, embryonic stem cell, adult stem cell, bone-marrow stem cell, umbilical cord stem cells, or other stem cell.

In some embodiments, a genetically engineered cell is autologous. In some embodiments, a genetically engineered cell is allogeneic.

In some embodiments, the genetically engineered cell further comprises an immune receptor. In some embodiments, the immune receptor is a naturally-occurring immune receptor (e.g., the genetically engineered is an immune cell expressing an endogenous immune receptor). In some embodiments, the immune receptor is a naturally-occurring antigen receptor. In some embodiments, the immune receptor is selected from a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor.

In some embodiments, the immune receptor of the cell is a chimeric immune receptor. In some embodiments, the immune receptor is a chimeric antigen receptor. In some embodiments, the chimeric receptor inhibits immune receptor activation upon ligand binding.

In some embodiments, the genetically engineered cell is further engineered to express an exogenous immune receptor. For example, the genetically engineered cell can be engineered to express a chimeric immune receptor, such as a CAR. In another example, the genetically engineered cell can be engineered to express a naturally-occurring immune receptor exogenous to the engineered cell.

In some embodiments, the genetically engineered cell is engineered to express a chimeric inhibitory receptor and an exogenous immune receptor. The genetically engineered cell can be engineered to express both a chimeric inhibitory receptor and an exogenous immune receptor simultaneously (e.g., polynucleotides encoding each receptor are introduced simultaneously). The genetically engineered cell can be engineered to express both a chimeric inhibitory receptor and an exogenous immune receptor sequentially (e.g., first engineered to express either the chimeric inhibitory receptor and the exogenous immune receptor, then subsequently engineered to express the other receptor).

In some embodiments, ligand binding to a chimeric inhibitory receptor of the present disclosure and cognate immune receptor ligand binding to the immune receptor localizes the chimeric inhibitory receptor proximal to the immune receptor. In some embodiments, localization of the chimeric inhibitory receptor proximal to the immune receptor inhibits immune receptor activation. In some embodiments, immune receptor activation is T cell activation. For example, in the case of T cell signaling and/or activation, respective ligands binding to the chimeric inhibitory receptor and the immune receptor localizes the chimeric inhibitory receptor proximal to the immune receptor in an immunological synapse.

### Method of Production and Use

In another aspect, the present disclosure provides a method of preparing a genetically engineered cell (e.g., a genetically engineered immunomodulatory cell) expressing or capable of expressing a chimeric inhibitory receptor for experimental or therapeutic use. In another aspect, the present disclosure provides a method of preparing a genetically engineered cell (e.g., a genetically engineered immunomodulatory cell) expressing or capable of expressing a chimeric inhibitory receptor and an immune receptor for experimental or therapeutic use.

Ex vivo procedures for making therapeutic chimeric inhibitory receptor-engineered cells are well known in the art. For example, cells are isolated from a mammal (e.g., a human) and genetically engineered (i.e., transduced or transfected in vitro) with a vector expressing a chimeric inhibitory receptor disclosed herein. The chimeric inhibitory receptor-engineered cell can be administered to a mammalian recipient to provide a therapeutic benefit. The mammalian recipient may be a human and the chimeric inhibitory receptor-modified cell can be autologous with respect to the recipient. Alternatively, the cells can be allogeneic, syngeneic or xenogeneic with respect to the recipient. The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In some embodiments, the methods comprise culturing the population of cells (e.g. in cell culture media) to a desired cell density (e.g., a cell density sufficient for a particular cell-based therapy). In some embodiments, the population of cells are cultured in the absence of an agent that represses activity of the repressible protease or in the presence of an agent that represses activity of the repressible protease.

In some embodiments, the population of cells is cultured for a period of time that results in the production of an expanded cell population that comprises at least 2-fold the number of cells of the starting population. In some embodiments, the population of cells is cultured for a period of time that results in the production of an expanded cell population that comprises at least 4-fold the number of cells of the starting population. In some embodiments, the population of cells is cultured for a period of time that results in the production of an expanded cell population that comprises at least 16-fold the number of cells of the starting population.

Also provided herein are methods of inhibiting immune receptor activation. In some embodiments, the method including: contacting a genetically engineered cell comprising a nucleic acid encoding at least one chimeric receptor of the present disclosure, a genetically engineered cell that express a chimeric inhibitory receptor of the present disclosure, or a pharmaceutical composition including the genetically engineered cell with a cognate ligand under conditions suitable for the chimeric inhibitory receptor to bind the cognate ligand, wherein, when localized proximal to an immune receptor expressed on a cell membrane of the engineered cell, the chimeric inhibitory inhibits immune receptor activation.

Also provided herein are method for reducing an immune response. In some embodiments, the method comprises: administering a genetically engineered cell comprising a nucleic acid encoding at least one chimeric receptor of the present disclosure, a genetically engineered cell that express a chimeric inhibitory receptor of the present disclosure, or a pharmaceutical composition including the genetically engineered cells to a subject in need of such treatment.

Also provided herein are method of preventing, attenuating, or inhibiting a cell-mediated immune response induced by a tumor-targeting chimeric receptor expressed on the surface of an immunomodulatory cell, the method including: administering a genetically engineered immunomodulatory cell comprising a nucleic acid encoding at least one chimeric receptor of the present disclosure, a genetically engineered immunomodulatory cell that express a chimeric inhibitory receptor of the present disclosure, or a pharmaceutical composition including the genetically engineered immunomodulatory cell to a subject in need of such treatment.

Also provided herein are method of preventing, attenuating, or inhibiting a cell-mediated immune response induced by a tumor-targeting chimeric receptor expressed on the surface of an immunomodulatory cell, the method including: contacting a genetically engineered immunomodulatory cell comprising a nucleic acid encoding at least one chimeric receptor of the present disclosure, a genetically engineered immunomodulatory cell that express a chimeric inhibitory receptor of the present disclosure, or a pharmaceutical composition including the genetically engineered immunomodulatory cell with a cognate ligand under conditions suitable for the chimeric inhibitory receptor to bind the cognate ligand, wherein, when localized proximal to an immune receptor expressed on a cell membrane of the engineered cell, the chimeric inhibitory inhibits immune receptor activation.

Also provided herein are methods of treating an autoimmune disease or disease treatable by reducing an immune response. In some embodiments, the method includes: administering a genetically engineered cell comprising a nucleic acid encoding at least one chimeric receptor of the present disclosure, genetically engineered cells of the present disclosure that express a chimeric inhibitory receptor, or a pharmaceutical composition including the genetically engineered cell to a subject in need of such treatment.

In some embodiments, the methods include administering or contacting genetically engineered cells that further express or are capable of expressing an immune receptor. In some embodiments, the methods include administering or contacting genetically engineered cells that are further engineered to express an immune receptor. In some embodiments, the methods include administering or contacting genetically engineered cells that further express or are capable of expressing a chimeric immune receptor. In some embodiments, the methods include administering or contacting genetically engineered cells that are further engineered to express a chimeric immune receptor. In some embodiments, the methods include administering or contacting genetically engineered cells that further express or are capable of expressing a CAR. In some embodiments, the methods include administering or contacting genetically engineered cells that are further engineered to express a CAR.

Attenuation of an immune response initiated by an immune receptor (e.g., a tumor targeting chimeric receptor) can be a decrease or reduction in the activation of the immune receptor, a decrease or reduction in the signal transduction of the immune receptor, or a decrease or reduction in the activation of the engineered cell. The inhibitory chimeric receptor can attenuate activation of the immune receptor, signal transduction by the immune receptor, or activation of the engineered cell by the immune receptor 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the immune receptor, signal transduction, or activation of the engineered cell as compared to an engineered cell lacking an inhibitory chimeric receptor. In some embodiments, attenuation refers to a decrease or reduction of the activity of the immune receptor after it has been activated.

Prevention of an immune response initiated by an immune receptor (e.g., a tumor targeting chimeric receptor) can be an inhibition or reduction in the activation of the immune receptor, an inhibition or reduction in the signal transduction of the immune receptor, or an inhibition or reduction in the activation of the engineered cell. The inhibitory chimeric receptor can prevent activation of the immune receptor, signal transduction by the immune receptor, or activation of the engineered cell by the immune receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the immune receptor, signal transduction, or activation of the engineered cell as compared to an engineered cell lacking an inhibitory chimeric receptor. In some embodiments, prevention refers to a blockage of the activity of the immune receptor before it has been activated.

Inhibition of an immune response initiated by an immune receptor (e.g., a tumor targeting chimeric receptor) can be an inhibition or reduction in the activation of the immune receptor, an inhibition or reduction in the signal transduction of the immune receptor, or an inhibition or reduction in the activation of the engineered cell. The inhibitory chimeric receptor can inhibit activation of the immune receptor, signal transduction by the immune receptor, or activation of the engineered cell by the immune receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the immune receptor, signal transduction, or activation of the engineered cell as compared to an engineered cell lacking an inhibitory chimeric receptor. In some embodiments, inhibition refers to a decrease or reduction of the activity of the immune receptor before or after it has been activated.

Suppression of an immune response initiated by an immune receptor (e.g., a tumor targeting chimeric receptor) can be an inhibition or reduction in the activation of the immune receptor, an inhibition or reduction in the signal transduction of the immune receptor, or an inhibition or reduction in the activation of the engineered cell. The inhibitory chimeric receptor can suppress activation of the immune receptor, signal transduction by the immune receptor, or activation of the engineered cell by the immune receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or more as compared to the activation of the immune receptor, signal transduction, or activation of the engineered cell as compared to an engineered cell lacking an inhibitory chimeric receptor. In some embodiments, suppression refers to a decrease or reduction of the activity of the immune receptor before or after it has been activated.

The immune response can be cytokine or chemokine production and secretion from an activated immunomodulatory cell. The immune response can be a cell-mediated immune response to a target cell, such as cell-mediated killing.

In some embodiments, the chimeric inhibitory receptor is capable of suppressing cytokine production from an activated engineered cell, such as an immunomodulatory cell. In some embodiments, the chimeric inhibitory receptor is capable of suppressing a cell-mediated immune response to a target cell, wherein the immune response is induced by activation of the engineered cell.

In one aspect, the present disclosure provides a type of cell therapy where cells, such as immune cells, are genetically engineered to express a chimeric inhibitory receptor provided herein and the genetically engineered cells are administered to a subject in need thereof.

Thus, in some embodiments, the methods comprise delivering cells of the expanded population of cells to a subject in need of a cell-based therapy to treat a condition or disorder. In some embodiments, the subject is a human subject. In some embodiments, the condition or disorder is an autoimmune condition. In some embodiments, the condition or disorder is an immune related condition. In some embodiments, the condition or disorder is a cancer (e.g., a primary cancer or a metastatic cancer). In some embodiments, the cancer is a solid cancer. In some embodiments, the cancer is a liquid cancer.

### Pharmaceutical Compositions

The chimeric inhibitory receptor or genetically engineered cell can be formulated in pharmaceutical compositions. Pharmaceutical compositions of the present disclosure can comprise a chimeric inhibitory receptor (e.g., an iCAR) or genetically engineered cell (e.g., a plurality of chimeric inhibitory receptor-expressing cells), as described herein, in combination with one or more pharmaceutically or physiologically acceptable carriers, diluents or excipients. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. In certain embodiments, the composition is directly injected into an organ of interest (e.g., an organ affected by a disorder). Alternatively, the composition may be provided indirectly to the organ of interest, for example, by administration into the circulatory system (e.g., the tumor vasculature). Expansion and differentiation agents can be provided prior to, during, or after administration of the composition to increase production of T cells, NK cells, or CTL cells in vitro or in vivo.

In certain embodiments, the compositions are pharmaceutical compositions comprising genetically engineered cells, such as immunomodulatory or immune cells, or their progenitors and a pharmaceutically acceptable carrier. Administration can be autologous or heterologous. For example, immunomodulatory or immune cells, or progenitors, can be obtained from one subject, and administered to the same subject or a different, compatible subject. In some embodiments, genetically engineered cells, such as immunomodulatory or immune cells, or their progeny may be derived from peripheral blood cells (e.g., in vivo, ex vivo, or in vitro derived) and may be administered via localized injection, including catheter administration, systemic injection, localized injection, intravenous injection, or parenteral administration. When administering a therapeutic composition of the present disclosure (e.g., a pharmaceutical composition containing a genetically engineered cell of the present disclosure), it will generally be formulated in a unit dosage injectable form (solution, suspension, emulsion).

Certain aspects of the present disclosure relate to formulations of compositions comprising chimeric receptors of the present disclosure or genetically engineered cells (e.g., immunomodulatory or immune cells of the present disclosure) expressing such chimeric receptors. In some embodiments, compositions of the present disclosure comprising genetically engineered cells may be provided as sterile liquid preparations, including without limitation isotonic aqueous solutions, suspensions, emulsions, dispersions, and viscous compositions, which may be buffered to a selected pH. Liquid preparations are typically easier to prepare than gels, other viscous compositions, and solid compositions. Additionally, liquid compositions may be more convenient to administer, especially by injection. In some embodiments, viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (e.g., glycerol, propylene glycol, liquid polyethylene glycol, etc.) and suitable mixtures thereof.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. In some embodiments, compositions of the present disclosure can be isotonic, i.e., having the same osmotic pressure as blood and lacrimal fluid. In some embodiments, the desired isotonicity may be achieved using, for example, sodium chloride, dextrose, boric acid, sodium tartrate, propylene glycol, or other inorganic or organic solutes.

In some embodiments, compositions of the present disclosure may further include various additives that may enhance the stability and sterility of the compositions. Examples of such additives include, without limitation, antimicrobial preservatives, antioxidants, chelating agents, and buffers. In some embodiments, microbial contamination may be prevented by the inclusions of any of various antibacterial and antifungal agents, including without limitation parabens, chlorobutanol, phenol, sorbic acid, and the like. Prolonged absorption of an injectable pharmaceutical formulation of the present disclosure can be brought about by the use of suitable agents that delay absorption, such as aluminum monostearate and gelatin. In some embodiments, sterile injectable solutions can be prepared by incorporating genetically modified cells of the present disclosure in a sufficient amount of the appropriate solvent with various amounts of any other ingredients, as desired. Such compositions may be in admixture with a suitable carrier, diluent, or excipient such as sterile water, physiological saline, glucose, dextrose, or the like. In some embodiments, the compositions can also be lyophilized. The compositions can contain auxiliary substances such as wetting, dispersing agents, pH buffering agents, and antimicrobials depending upon the route of administration and the preparation desired.

In some embodiments, the components of the formulations of the present disclosure are selected to be chemically inert and to not affect the viability or efficacy of the genetically modified cells of the present disclosure.

One consideration concerning the therapeutic use of the genetically engineered cells of the present disclosure is the quantity of cells needed to achieve optimal efficacy. In some embodiments, the quantity of cells to be administered will vary for the subject being treated. In certain embodiments, the quantity of genetically engineered cells that are administered to a subject in need thereof may range from 1×10⁴ cells to 1×10¹⁰ cells. In some embodiments, the precise quantity of cells that would be considered an effective dose may be based on factors individual to each subject, including their size, age, sex, weight, and condition of the particular subject. Dosages can be readily ascertained by those skilled in the art based on the present disclosure and the knowledge in the art.

Whether it is a polypeptide, antibody, nucleic acid, small molecule or other pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Kits

Certain aspects of the present disclosure relate to kits for the treatment and/or prevention of a cancer or other diseases (e.g., immune-related or autoimmune disorders). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., genetically engineered cells, such as immunomodulatory or immune cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing a cancer or immune-related disorder. In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### Additional Embodiments

Provided below are enumerated embodiments describing specific non-limiting embodiments of the present invention:

Embodiment 1. A chimeric inhibitory receptor comprising:

an extracellular ligand binding domain;

a membrane localization domain, wherein the membrane localization domain comprises a transmembrane domain; and

an enzymatic inhibitory domain, wherein the enzymatic inhibitory domain inhibits immune receptor activation when proximal to an immune receptor.

Embodiment 2. The chimeric inhibitory receptor of embodiment 1, wherein the extracellular ligand binding domain binds to a ligand selected from the group consisting of: a protein complex, a protein, a peptide, a receptor-binding domain, a nucleic acid, a small molecule, and a chemical agent.

Embodiment 3. The chimeric inhibitory receptor of embodiment 1 or embodiment 2, wherein the extracellular ligand binding domain comprises an antibody, or antigen-binding fragment thereof.

Embodiment 4. The chimeric inhibitory receptor of embodiment 1 or embodiment 2, wherein the extracellular ligand binding domain comprises a F(ab) fragment, a F(ab′) fragment, a single chain variable fragment (scFv), or a single-domain antibody (sdAb).

Embodiment 5. The chimeric inhibitory receptor of any one of embodiments 1-4, wherein the ligand is a tumor-associated antigen.

Embodiment 6. The chimeric inhibitory receptor of any one of embodiments 1-4, wherein the ligand is not expressed on a tumor cell.

Embodiment 7. The chimeric inhibitory receptor of any one of embodiments 1-4, wherein the ligand is expressed on a non-tumor cell.

Embodiment 8. The chimeric inhibitory receptor of any one of embodiments 1-4, wherein the ligand is expressed on cells of a healthy tissue.

Embodiment 9. The chimeric inhibitory receptor of any one of embodiments 1-8, wherein the extracellular ligand binding domain comprises a dimerization domain.

Embodiment 10. The chimeric inhibitory receptor of embodiment 9, wherein the ligand further comprises a cognate dimerization domain.

Embodiment 11. The chimeric inhibitory receptor of any one of embodiments 2-10, wherein the ligand is a cell surface ligand.

Embodiment 12. The chimeric inhibitory receptor of embodiment 11, wherein the cell surface ligand is expressed on a cell that further expresses a cognate ligand of the immune receptor.

Embodiment 13. The chimeric inhibitory receptor of any one of embodiments 1-12, wherein the membrane localization domain further comprises at least a portion of an extracellular domain.

Embodiment 14. The chimeric inhibitory receptor of any one of embodiments 1-12, wherein the membrane localization domain further comprises at least a portion of an intracellular domain.

Embodiment 15. The chimeric inhibitory receptor of any one of embodiments 1-12, wherein the membrane localization domain further comprises at least a portion of an extracellular domain and at least a portion of an intracellular domain.

Embodiment 16. The chimeric inhibitory receptor of any one of embodiments 1-12, wherein the membrane localization domain comprises a transmembrane domain selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CD8 transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAGS transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.

Embodiment 17. The chimeric inhibitory receptor of embodiment 16, wherein the membrane localization domain further comprises at least a portion of a corresponding extracellular domain and/or at least a portion of a corresponding intracellular domain.

Embodiment 18. The chimeric inhibitory receptor of embodiment 16 or embodiment 17, wherein the LAT mutant is a LAT(CA) mutant.

Embodiment 19. The chimeric inhibitory receptor of any one of embodiments 1-18, wherein the membrane localization domain directs or segregates the chimeric inhibitory receptor to a domain of a cell membrane.

Embodiment 20. The chimeric inhibitory receptor of any one of embodiments 1-19, wherein the membrane localization domain localizes the chimeric inhibitory receptor to a lipid raft or a heavy lipid raft.

Embodiment 21. The chimeric inhibitory receptor of any one of embodiments 1-20, wherein the membrane localization domain interacts with one or more cell membrane components localized in a domain of a cell membrane.

Embodiment 22. The chimeric inhibitory receptor of any one of embodiments 1-21, wherein the membrane localization domain is sufficient to mitigate constitutive inhibition of immune receptor activation by the enzymatic inhibitory domain in the absence of the extracellular ligand binding domain binding a cognate ligand.

Embodiment 23. The chimeric inhibitory receptor of any one of embodiments 1-21, wherein the membrane localization domain mediates localization of the chimeric inhibitory receptor to a domain of a cell membrane that is distinct from domains of the cell membrane occupied by one or more components of an immune receptor in the absence of the extracellular ligand binding domain binding a cognate ligand.

Embodiment 24. The chimeric inhibitory receptor of embodiment 23, wherein the membrane localization domain further comprises proximal protein fragments.

Embodiment 25. The chimeric inhibitory receptor of any one of embodiments 1-24, wherein the chimeric inhibitory receptor further comprises one or more intracellular inhibitory co-signaling domains.

Embodiment 26. The chimeric inhibitory receptor of embodiment 25, wherein the one or more intracellular inhibitory co-signaling domains comprise one or more ITIM-containing proteins, or fragments thereof.

Embodiment 27. The chimeric inhibitory receptor of embodiment 26, wherein the one or more ITIM-containing proteins, or fragments thereof, are selected from the group consisting of: PD-1, CTLA4, TIGIT, BTLA, and LAIR1.

Embodiment 28. The chimeric inhibitory receptor of embodiment 25, wherein the one or more intracellular inhibitory co-signaling domains comprise one or more non-ITIM scaffold proteins, or fragments thereof.

Embodiment 29. The chimeric inhibitory receptor of embodiment 28, wherein the one or more non-ITIM scaffold proteins, or fragments thereof, are selected from the group consisting of: GRB-2, Dok-1, Dok-2, SLAP1, SLAP2, LAGS, HAVR, GITR, and PD-L1.

Embodiment 30. The chimeric inhibitory receptor of any one of embodiments 1-29, wherein the extracellular ligand binding domain is linked to the membrane localization domain through an extracellular linker region.

Embodiment 31. The chimeric inhibitory receptor of embodiment 30, wherein the extracellular linker region is positioned between the extracellular ligand binding domain and membrane localization domain and operably and/or physically linked to each of the extracellular ligand binding domain and the membrane localization domain.

Embodiment 32. The chimeric inhibitory receptor of embodiment 30 or embodiment 31, wherein the extracellular linker region is derived from a protein selected from the group consisting of: CD8alpha, CD4, CD7, CD28, IgG1, IgG4, FcgammaRIIIalpha, LNGFR, and PDGFR.

Embodiment 33. The chimeric inhibitory receptor of embodiment 30 or embodiment 31, wherein the extracellular linker region comprises an amino acid sequence selected from the group consisting of: AAAIEVMYPPPYLDNEKSNGTIIHVKGKHLCPSPLFPGPSKP (SEQ ID NO:46), ESKYGPPCPSCP (SEQ ID NO:47), ESKYGPPAPSAP (SEQ ID NO:48), ESKYGPPCPPCP (SEQ ID NO:49), EPKSCDKTHTCP (SEQ ID NO:50), AAAFVPVFLPAKPTTTPAPRPPTPAPTIAS QPLSLRPEACRPAAGGAVHTRGLDFACDI YIWAPLAGTCGVLLLSLVITLYCNHRN (SEQ ID NO:51), TTTPAPRPPTPAPTIALQPLSLRPEACRPAAGGAVHTRGLDFACD (SEQ ID NO:52), ACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQ NTVCEECPDGTYSDEADAEC (SEQ ID NO:53), ACPTGLYTHSGECCKACNLGEGVAQPCGANQTVC (SEQ ID NO:54), and AVGQDTQEVIVVPHSLPFKV (SEQ ID NO:55).

Embodiment 34. The chimeric inhibitory receptor of embodiment 30 or embodiment 31, wherein the extracellular linker region comprises an amino acid sequence selected from the group consisting of: GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), GSTSGSGKPGSGEGSTKG (SEQ ID NO: 44), and EAAAKEAAAKEAAAKEAAAK (SEQ ID NO: 45).

Embodiment 35. The chimeric inhibitory receptor of any one of embodiments 1-33, wherein the chimeric inhibitory receptor further comprises an intracellular spacer region positioned between the membrane localization domain and the enzymatic inhibitory domain and operably and/or physically linked to each of the membrane localization domain and the enzymatic inhibitory domain.

Embodiment 36. The chimeric inhibitory receptor of embodiment 34, wherein the intracellular spacer region comprises an amino acid sequence selected from the group consisting of: GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), GSTSGSGKPGSGEGSTKG (SEQ ID NO: 44), and EAAAKEAAAKEAAAKEAAAK (SEQ ID NO: 45).

Embodiment 37. The chimeric inhibitory receptor of embodiment 34, wherein the intracellular spacer region comprises an amino acid sequence selected from the group consisting of: AAAIEVMYPPPYLDNEKSNGTIIHVKGKHLCPSPLFPGPSKP (SEQ ID NO:46), ESKYGPPCPSCP (SEQ ID NO:47), ESKYGPPAPSAP (SEQ ID NO:48), ESKYGPPCPPCP (SEQ ID NO:49), EPKSCDKTHTCP (SEQ ID NO:50), AAAFVPVFLPAKPTTTPAPRPPTPAPTIAS QPLSLRPEACRPAAGGAVHTRGLDFACDI YIWAPLAGTCGVLLLSLVITLYCNHRN (SEQ ID NO:51), TTTPAPRPPTPAPTIALQPLSLRPEACRPAAGGAVHTRGLDFACD (SEQ ID NO:52), ACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQ NTVCEECPDGTYSDEADAEC (SEQ ID NO:53), ACPTGLYTHSGECCKACNLGEGVAQPCGANQTVC (SEQ ID NO:54), and AVGQDTQEVIVVPHSLPFKV (SEQ ID NO:55).

Embodiment 38. The chimeric inhibitory receptor of any one of embodiments 1-34, wherein the enzymatic inhibitory domain comprises at least a portion of an extracellular domain, a transmembrane domain, and/or an intracellular domain.

Embodiment 39. The chimeric inhibitory receptor of embodiment 38, wherein the enzymatic inhibitory domain comprises an enzyme catalytic domain.

Embodiment 40. The chimeric inhibitory receptor of any one of embodiments 1-34, wherein the enzymatic inhibitory domain comprises at least a portion of an enzyme.

Embodiment 41. The chimeric inhibitory receptor of embodiment 40, wherein the portion of the enzyme comprises an enzyme domain or an enzyme fragment.

Embodiment 42. The chimeric inhibitory receptor of embodiment 40, wherein the portion of the enzyme is a catalytic domain of the enzyme.

Embodiment 43. The chimeric inhibitory receptor of any one of embodiments 39-42, wherein the enzyme is selected from the group consisting of: CSK, SHP-1, SHP-2, PTEN, CD45, CD148, PTP-MEG1, PTP-PEST, c-CBL, CBL-b, PTPN22, LAR, PTPH1, SHIP-1, ZAP70, and RasGAP.

Embodiment 44. The chimeric inhibitory receptor of any one of embodiments 1-43, wherein the enzymatic inhibitory domain is derived from CSK.

Embodiment 45. The chimeric inhibitory receptor of embodiment 44, wherein the enzymatic inhibitory domain comprises a CSK protein with a SRC homology 3 (SH3) deletion.

Embodiment 46. The chimeric inhibitory receptor of any one of embodiments 1-43, wherein the enzymatic inhibitory domain is derived from SHP-1.

Embodiment 47. The chimeric inhibitory receptor of embodiment 47, wherein the enzymatic inhibitory domain comprises a protein tyrosine phosphatase (PTP) domain.

Embodiment 48. The chimeric inhibitory receptor of any one of embodiments 1-43, wherein the enzymatic inhibitory domain is derived from SHP-2.

Embodiment 49. The chimeric inhibitory receptor of any one of embodiments 1-43, wherein the enzymatic inhibitory domain is derived from PTEN.

Embodiment 50. The chimeric inhibitory receptor of any one of embodiments 1-43, wherein the enzymatic inhibitory domain is derived from CD45.

Embodiment 51. The chimeric inhibitory receptor of any one of embodiments 1-43, wherein the enzymatic inhibitory domain is derived from CD148.

Embodiment 52. The chimeric inhibitory receptor of any one of embodiments 1-43, wherein the enzymatic inhibitory domain is derived from PTP-MEG1.

Embodiment 53. The chimeric inhibitory receptor of any one of embodiments 1-43, wherein the enzymatic inhibitory domain is derived from PTP-PEST.

Embodiment 54. The chimeric inhibitory receptor of any one of embodiments 1-43, wherein the enzymatic inhibitory domain is derived from c-CBL.

Embodiment 55. The chimeric inhibitory receptor of any one of embodiments 1-43, wherein the enzymatic inhibitory domain is derived from CBL-b.

Embodiment 56. The chimeric inhibitory receptor of any one of embodiments 1-43, wherein the enzymatic inhibitory domain is derived from PTPN22.

Embodiment 57. The chimeric inhibitory receptor of any one of embodiments 1-43, wherein the enzymatic inhibitory domain is derived from LAR.

Embodiment 58. The chimeric inhibitory receptor of any one of embodiments 1-43, wherein the enzymatic inhibitory domain is derived from PTPH1.

Embodiment 59. The chimeric inhibitory receptor of any one of embodiments 1-43, wherein the enzymatic inhibitory domain is derived from SHIP-1.

Embodiment 60. The chimeric inhibitory receptor of embodiment 60, wherein the enzymatic inhibitory domain comprises a protein tyrosine phosphatase (PTP) domain.

Embodiment 61. The chimeric inhibitory receptor of any one of embodiments 1-43, wherein the enzymatic inhibitory domain is derived from ZAP70.

Embodiment 62. The chimeric inhibitory receptor of embodiment 58, wherein the enzymatic inhibitory domain comprises a SRC homology 1 (SH1) domain, a SRC homology 2 (SH2) domain, or an SH1 domain and an SH2 domain.

Embodiment 63. The chimeric inhibitory receptor of embodiment 58, wherein the enzymatic inhibitory domain comprises a ZAP70 protein with a kinase domain deletion.

Embodiment 64. The chimeric inhibitory receptor of embodiment 58, wherein the enzymatic inhibitory domain comprises a mutant ZAP70 protein with a Tyr492Phe amino acid substitution, a Tyr493Phe amino acid substitution, or a Tyr492Phe amino acid substitution and a Tyr493Phe amino acid substitution.

Embodiment 65. The chimeric inhibitory receptor of any one of embodiments 1-43, wherein the enzymatic inhibitory domain is derived from RasGAP.

Embodiment 66. The chimeric inhibitory receptor of any one of embodiments 1-43, wherein the enzymatic inhibitory domain comprises one or more modifications that modulate basal inhibition.

Embodiment 67. The chimeric inhibitory receptor of embodiment 65, wherein the one or more modifications reduce basal inhibition.

Embodiment 68. The chimeric inhibitory receptor of embodiment 65, wherein the one or more modifications increase basal inhibition.

Embodiment 69. The chimeric inhibitory receptor of any one of embodiments 1-68, wherein the enzymatic inhibitory domain inhibits immune receptor activation upon recruitment of the chimeric inhibitory receptor proximal to an immune receptor.

Embodiment 70. The chimeric inhibitory receptor of any one of embodiments 1-69, wherein the immune receptor is a chimeric immune receptor.

Embodiment 71. The chimeric inhibitory receptor of embodiment 70, wherein the immune receptor is a chimeric antigen receptor.

Embodiment 72. The chimeric inhibitory receptor of any one of embodiments 1-69, wherein the immune receptor is a naturally-occurring immune receptor.

Embodiment 73. The chimeric inhibitory receptor of embodiment 72, wherein the immune receptor is a naturally-occurring antigen receptor.

Embodiment 74. The chimeric inhibitory receptor of any one of embodiments 1-69, wherein the immune receptor is selected from the group consisting of: a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor.

Embodiment 75. The chimeric inhibitory receptor of any one of embodiments 1-73, wherein the immune receptor is a T cell receptor.

Embodiment 76. A nucleic acid encoding the chimeric inhibitory receptor of any one of embodiments 1-75.

Embodiment 77. A vector comprising the nucleic acid of embodiment 76.

Embodiment 78. A genetically engineered cell comprising the nucleic acid of embodiment 76.

Embodiment 79. A genetically engineered cell comprising the vector of embodiment 77.

Embodiment 80. A genetically engineered cell expressing the chimeric inhibitory receptor of any one of embodiments 1-75.

Embodiment 81. A genetically engineered cell expressing a chimeric inhibitory receptor, wherein the chimeric inhibitory receptor comprises:
an extracellular ligand binding domain;
a membrane localization domain, wherein the membrane localization domain comprises a transmembrane domain; and
an enzymatic inhibitory domain, wherein the inhibitory domain inhibits immune receptor activation when proximal to an immune receptor.

Embodiment 82. The engineered cell of any one of embodiments 78-81, wherein the cell further comprises an immune receptor.

Embodiment 83. The engineered cell of embodiment 82, wherein the immune receptor is a chimeric immune receptor.

Embodiment 84. The engineered cell of embodiment 83, wherein the immune receptor is a chimeric antigen receptor.

Embodiment 85. The engineered cell of embodiment 82, wherein the immune receptor is a naturally-occurring immune receptor.

Embodiment 86. The engineered cell of embodiment 85, wherein the immune receptor is a naturally-occurring antigen receptor.

Embodiment 87. The engineered cell of embodiment 82, wherein the immune receptor is selected from the group consisting of: a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor.

Embodiment 88. The engineered cell of any one of embodiments 82-87, wherein the chimeric inhibitory receptor inhibits immune receptor activation upon ligand binding.

Embodiment 89. The engineered cell of any one of embodiments 82-88, wherein the ligand is a cell surface ligand.

Embodiment 90. The engineered cell of embodiment 89, wherein the cell surface ligand is expressed on a cell that further expresses a cognate immune receptor ligand.

Embodiment 91. The engineered cell of embodiment 90, wherein ligand binding to the chimeric inhibitory receptor and cognate immune receptor ligand binding to the immune receptor localizes the chimeric inhibitory receptor proximal to the immune receptor.

Embodiment 92. The engineered cell of embodiment 91, wherein localization of the chimeric inhibitory receptor proximal to the immune receptor inhibits immune receptor activation.

Embodiment 93. The engineered cell of any one of embodiments 88-93, wherein the cell is a T cell.

Embodiment 94. The engineered cell of embodiment 93, wherein the immune receptor is a T cell receptor.

Embodiment 95. The engineered cell of embodiment 94, wherein immune receptor activation is T cell activation.

Embodiment 96. The engineered cell of any one of embodiments 78-92, wherein the cell is an immunomodulatory cell.

Embodiment 97. The engineered cell of embodiment 96, wherein the immunomodulatory cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

Embodiment 98. The engineered cell of of any one of embodiments 78-97, wherein the cell is autologous.

Embodiment 99. The engineered cell of of any one of embodiments 78-97, wherein the cell is allogeneic.

Embodiment 100. A pharmaceutical composition comprising the engineered cell of any one of embodiments 78-99 and a pharmaceutically acceptable carrier, a pharmaceutically acceptable excipient, or combination thereof.

Embodiment 101. A method of inhibiting immune receptor activation, comprising:

contacting the engineered cell of any one of embodiments 78-99 or the pharmaceutical composition of embodiment 100 with a cognate ligand under conditions suitable for the chimeric inhibitory receptor to bind the cognate ligand,
wherein, when localized proximal to an immune receptor expressed on a cell membrane of the engineered cell, the chimeric inhibitory inhibits immune receptor activation.

Embodiment 102. A method for reducing an immune response, comprising:

administering the engineered cell of any one of embodiments 78-99 or the pharmaceutical composition of embodiment 100 to a subject in need of such treatment.

Embodiment 103. A method of preventing, attenuating, or inhibiting a cell-mediated immune response induced by a tumor-targeting chimeric receptor expressed on the surface of an immunomodulatory cell, comprising:

administering the engineered cell of any one of embodiments 78-99 or the pharmaceutical composition of embodiment 100 to a subject in need of such treatment.

Embodiment 104. A method of preventing, attenuating, or inhibiting activation of a tumor-targeting chimeric receptor expressed on the surface of an immunomodulatory cell, comprising:

contacting the engineered cell of any one embodiments 78-99 or the pharmaceutical composition of embodiment 100 with a cognate ligand of the chimeric inhibitory receptor under conditions suitable for the chimeric inhibitory receptor to bind the cognate ligand,
wherein upon binding of the ligand to the chimeric inhibitory receptor, the enzymatic inhibitory domain prevents, attenuates, or inhibits activation of the tumor-targeting chimeric receptor.

Embodiment 105. A method for treating an autoimmune disease or disease treatable by reducing an immune response comprising:

administering the engineered cell of any one of embodiments 78-99 or the pharmaceutical composition of embodiment 100 to a subject in need of such treatment.

### EXAMPLES

The following are examples of methods and compositions of the present disclosure. It is understood that various other embodiments may be practiced, given the general description provided herein.

Below are examples of specific embodiments for carrying out the claimed subject matter of the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

### Example 1: Inhibition by Enzymatic Inhibitory Domain (EID)-Containing CAR CAR-T and K562 Co-Culture Methods

### Lentiviral Production:

Lentivirus was produced using: Lenti-X 293T packaging cell line (Clontech, Cat #632180); LX293T Complete growth medium, without antibiotics; DMEM, hi-glucose; 1 mM Sodium Pyruvate; 10% FBS, heat-inactivated; Opti-Mem I Reduced Serum Media (Gibco/Thermo Fisher; Cat #31985); FuGene HD (Promega, Cat #E2311); Envelope, Packaging, and Transfer Vector plasmids; VSV-G-pseudotyped envelope vector (pMD2.G); Packaging vector that contains Gag, Pol, Rev, and Tat that can be used with 2nd and 3rd generation transfer vectors (psMAX2). 293T(FT) cells from 90% confluent 10 cm dishes were lifted and dispensed at 1:3 dilution late in the afternoon the day before transfection and incubated cells at normal overnight at 37° C., 5% CO2 (cells should be 60-85% confluent the next day at time of transfection).

A transfection reaction was prepped for each 10 cm dish according to the protocol below:
1. Prep transfection reaction for each 10 cm dish in a separate 1.7 mL tube.
2. Add 900 uL Opti-Mem I at RT.
3. Add 9 ug vector backbone (containing gene of interest) per reaction.
4. Add 8 ug packaging vector per reaction.
5. Add 1 ug envelope vector per reaction (pMD2.G).
6. Mix thoroughly by quickly vortexing for 3 seconds.
7. Add 55 uL Fugene HD per reaction.
8. Mix by quickly pipetting up and down 20-30 times.
9. Let sit at RT for 10 min (allowing DNA complexes to form).
10. Slowly add mixture in dropwise manner around the dish, then mix by gently rocking back-forth and up-down for 5-10 seconds (do not swirl).
11. Place dish into virus incubator.

Viral supernatants were harvested on days 2 and 3 using a serological pipette. Cellular debris was removed using a Millipore steriflip 0.45 um filters. A Lenti-X Concentrator (Cat. Nos. 631231 & 631232) was used according to the protocol: 1) Combine 1 volume of Lenti-X Concentrator with 3 volumes of clarified supernatant. Mix by gentle inversion; 2) Incubate mixture on ice or at 4° C. for 30 minutes to overnight; (3) Centrifuge sample at 1,500×g for 45 minutes at 4° C.; (4) Carefully remove and discard supernatant, taking care not to disturb the pellet; (5) Gently resuspend the pellet in 1/10 to 1/100th of the original volume using sterile PBS+0.1% BSA.

### Transduction and Expansion

Primary T cells were isolated from human donor PBMCs and frozen. On Day 1, 1×10⁶ purified CD4+/CD8+ T-cells were thawed and stimulated with 3×10⁶ Human T-Activator CD3/CD28 Dynabeads, then cultured in 1 mL Optimizer CTS T-cell expansion media (Gibco) with 0.2 μg/mL IL-2. On Day 2, cells were co-transduced with a lentivirus (see production methods above) encoding an activating CAR (aCAR) and/or a lentivirus encoding an inhibitory CAR (iCAR) to produce aCAR+, iCAR+, and aCAR+/iCAR+(dual+) T cells (100K each construct, as quantified by GoStix (Tekara)). Each CAR was under control of a constitutive SFFV promoter. The various aCAR and iCAR constructs and associated CAR domains are described below in Table A and the full coding sequences provided in Table C. On Day 3, Dynabeads were removed by magnet. T-cells were counted and passaged (0.5×10⁶ cells/mL). During subsequent expansion, cells were passaged every two days (0.5×10⁶ cells/mL).

TABLE A
CAR Constructs

| Construct | Promoter | Signal seq. | Tag | scFV | Hinge | TM | IC #1 | Marker |
|---|---|---|---|---|---|---|---|---|
| aCAR | SFFV | CD8 | | anti-CD20 | MYC-CD8 | CD28 | CD28- | |
| | | | | | | | CD3zeta | |
| iCAR17 | SFFV | IgGkappa | FLAG | anti-CD19 | CD8 | PAG | Csk | |
| iCAR25 | SFFV | IgGkappa | FLAG | anti-CD19 | CD8 | CD28 | Csk | 2A-PuroR |
| iCAR26 | SFFV | IgGkappa | FLAG | anti-CD19 | CD8 | CD28 | Csk_SH3 | 2A-PuroR |
| iCAR30 | SFFV | IgGkappa | FLAG | anti-CD19 | CD8 | LAT | Csk | 2A-PuroR |
| iCAR31 | SFFV | IgGkappa | Flag | anti-CD19 | CD8 | PAG | Csk | 2A-PuroR |

### Co-Culture Assay

On Day 7, an aliquot of each cell population was stained with PE conjugated anti-MYC and BV421 conjugated anti-FLAG antibodies (corresponding to aCAR and iCAR, respectively), and their transgene expression quantified using an LX CytoFlex Flow Cytometry machine. On Day 8, T-cells were counted and distributed into a 96-well plate for co-culture assays, with each well containing 5×10⁵ K562 target cells either engineered to co-express aCAR target CD20 and iCAR target CD19 or engineered to express CD20 alone and stained with CellTrace Violet dye (Invitrogen) and 5×10⁵ aCAR+ or dual+ T-cells. Co-cultures incubated (37°, 5% CO₂) for 40 hrs. On Day 10, cells in co-cultures were stained with NIR viability dye (Biolegend) and the number of live target cells was quantified using a CytoFlex LX flow cytometer. Killing efficiencies for each engineered CAR-T cell population were calculated as the ratio of surviving wild type K562 relative to each of the CD20-expressing K562 target cell lines. Normalized killing efficiencies were calculated as the ratio of CAR-T killing efficiencies for dual (CD20+CD19+) vs single (CD20+ only) antigen target cells.

### Enzymatic Inhibitory Domain Containing CAR Results

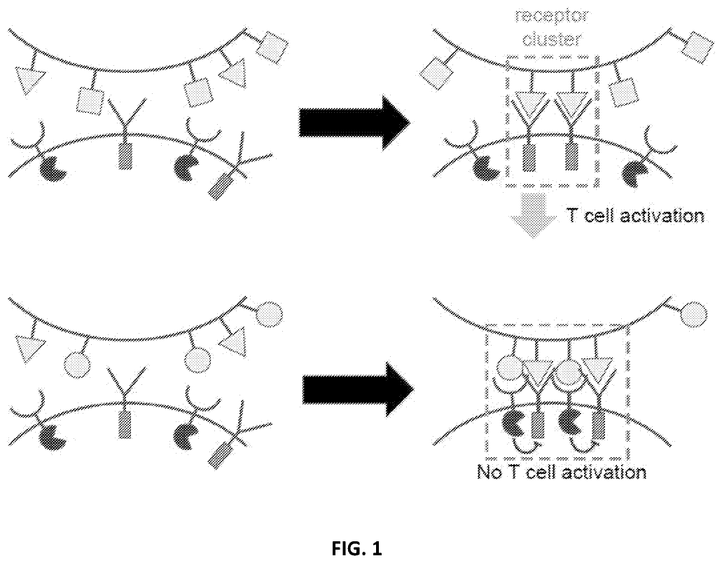

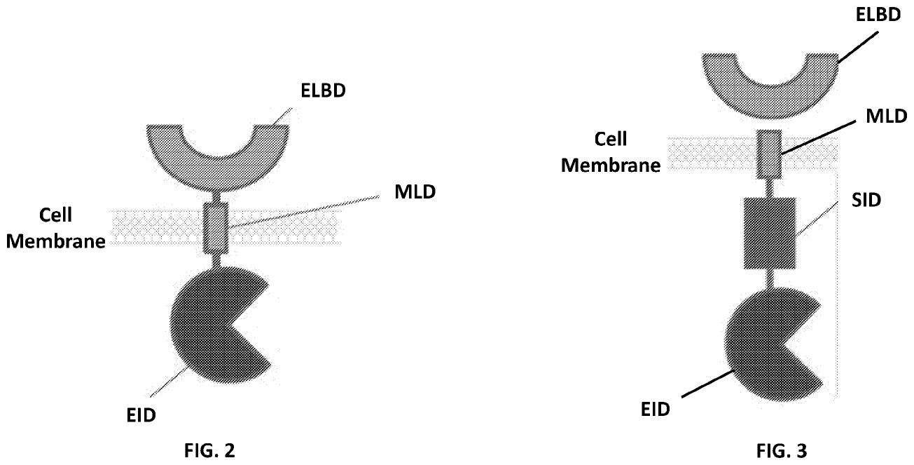

Inhibition of T cell signaling by a CAR containing an enzymatic inhibitory domain (EID) was assessed. The general strategy is schematized in FIGS. 1-3 showing inhibition of signaling mediated by EID-containing chimeric receptors when the receptor engages a cognate ligand expressed on a target cell.

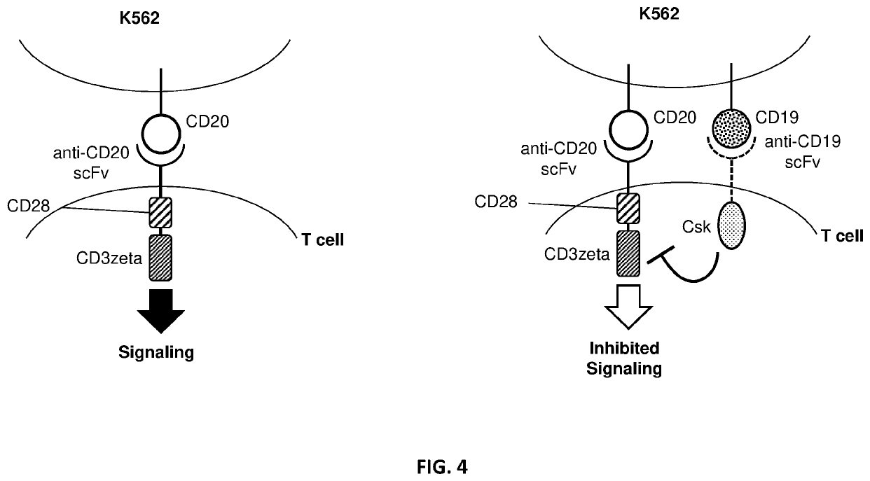

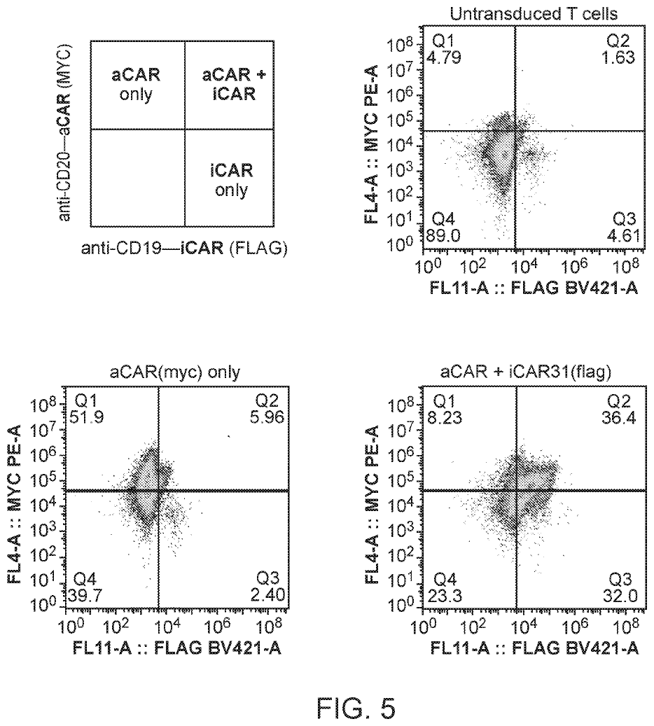

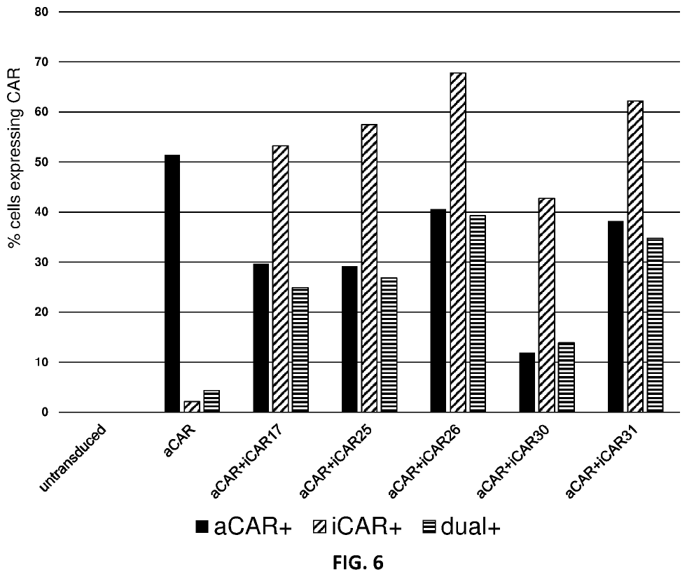

A system for assessing inhibition by EID-containing chimeric receptors was established. FIG. 4 schematizes the system where k562 target cells were engineered to express a cognate antigen for an aCAR (CD20) or engineered to express both the cognate antigen for the aCAR (CD20) and a cognate antigen for an iCAR (CD19). The system examined assessed the ability of an anti-CD19 iCAR including a CSK domain as the EID domain to inhibit signaling of an aCAR including a CD28-CD3ζ intracellular signaling domain. FIG. 5 provides representative flow-cytometry plots demonstrating the iCAR construct anti-CD19_scFv-Csk fusions was expressed at levels detectable above unmodified cells following transduction of CD4+ and CD8+ T cells without subsequent enrichment. Importantly, T cells demonstrated co-expression of both iCAR and aCAR constructs following lentiviral co-transduction (FIG. 5, bottom right). Expression profiles for the various constructs examined was assessed by flow-cytometry and presented in FIG. 6 demonstrating expression of the aCAR and iCAR constructs. Shown is: aCAR+=cells that express the aCAR (w/ and w/out iCAR) [first column]; iCAR+=cells that express the iCAR (w/ and w/out the aCAR) [second column]; and dual+=cells that express both the aCAR and iCAR [third column]. Importantly, a comparison of the aCAR+ population (first column) and dual+ population (third column) demonstrates the majority of the cells expressing an aCAR are dual+(i.e., also express an iCAR), indicating minimal residual aCAR-only cells (i.e., express only an aCAR) were present that would not be inhibited by a functional iCAR.

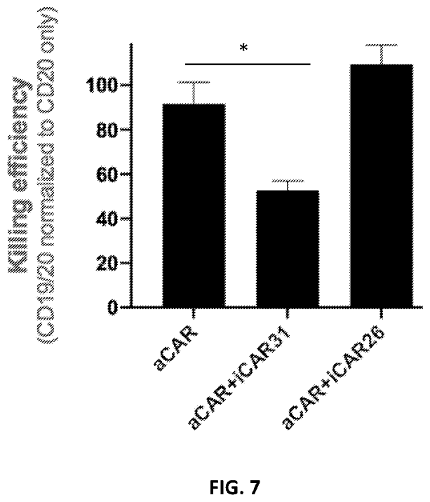

The iCAR constructs were then assessed for their ability to inhibit signaling. As shown in FIG. 7, while transduction with an aCAR construct only led to efficient target cell killing (FIG. 7, left column; represented as ratio of killing CD19/CD20 targets cells to CD20-only target cells), co-transduction of T cells with an iCAR possessing a CSK enzymatic inhibitory domain (iCAR31) led to an ˜50% reduction in killing efficiency (FIG. 7, middle column). Co-transduction of T cells with an iCAR possessing a CSK enzymatic inhibitory domain with a deletion in the CSK SH3 domain (iCAR26) did not demonstrate inhibition (FIG. 7, right column). Accordingly, the data demonstrate a CAR containing an enzymatic inhibitory domain was capable of inhibiting cellular signaling mediated by an activating CAR in a ligand-specific manner.

### Example 2: Assessment of Enzymatic Inhibitory Domain (EID)-Containing CARs

### CAR-T and K562 Co-Culture Methods

### Lentiviral Production:

Lentivirus is produced using: Lenti-X 293T packaging cell line (Clontech, Cat #632180); LX293T Complete growth medium, without antibiotics; DMEM, hi-glucose; 1 mM Sodium Pyruvate; 10% FBS, heat-inactivated; Opti-Mem I Reduced Serum Media (Gibco/Thermo Fisher; Cat #31985); FuGene HD (Promega, Cat #E2311); Envelope, Packaging, and Transfer Vector plasmids; VSV-G-pseudotyped envelope vector (pMD2.G); Packaging vector that contains Gag, Pol, Rev, and Tat that can be used with 2nd and 3rd generation transfer vectors (psMAX2). 293T(FT) cells from 90% confluent 10 cm dishes are lifted and dispensed at 1:3 dilution late in the afternoon the day before transfection and cells are incubated at normal overnight at 37° C., 5% CO₂ (cells should be 60-85% confluent the next day at time of transfection).

A transfection reaction is prepped for each 10 cm dish according to the protocol below:
1. Prep transfection reaction for each 10 cm dish in a separate 1.7 mL tube.
2. Add 900 uL Opti-Mem I at RT.
3. Add 9 ug vector backbone (containing gene of interest) per reaction.
4. Add 8 ug packaging vector per reaction.
5. Add 1 ug envelope vector per reaction (pMD2.G).
6. Mix thoroughly by quickly vortexing for 3 seconds.
7. Add 55 uL Fugene HD per reaction.
8. Mix by quickly pipetting up and down 20-30 times.
9. Let sit at RT for 10 min (allowing DNA complexes to form).
10. Slowly add mixture in dropwise manner around the dish, then mix by gently rocking back-forth and up-down for 5-10 seconds (do not swirl).
11. Place dish into virus incubator.

Viral supernatants are harvested on days 2 and 3 using a serological pipette. Cellular debris are removed using a Millipore steriflip 0.45 um filters. A Lenti-X Concentrator (Cat. Nos. 631231 & 631232) is used according to the protocol: 1) Combine 1 volume of Lenti-X Concentrator with 3 volumes of clarified supernatant. Mix by gentle inversion; 2) Incubate mixture on ice or at 4° C. for 30 minutes to overnight; (3) Centrifuge sample at 1,500×g for 45 minutes at 4° C.; (4) Carefully remove and discard supernatant, taking care not to disturb the pellet; (5) Gently resuspend the pellet in 1/10 to 1/100th of the original volume using sterile PBS+0.1% BSA.

### Transduction and Expansion

Primary T cells are isolated from human donor PBMCs and frozen. On Day 1, 1×10⁶ purified CD4+/CD8+ T-cells are thawed and stimulated with 3×10⁶ Human T-Activator CD3/CD28 Dynabeads, then cultured in 1 mL Optimizer CTS T-cell expansion media (Gibco) with 0.2 μg/mL IL-2. On Day 2, cells are co-transduced with a lentivirus (see production methods above) encoding an activating CAR (aCAR) and/or a lentivirus encoding an inhibitory CAR (iCAR) to produce aCAR+, iCAR+, and aCAR+/iCAR+(dual+) T cells (100K each construct, as quantified by GoStix (Tekara)). Each CAR is under control of a constitutive SFFV promoter. The various aCAR and iCAR constructs and associated CAR domains are described below in Table B. On Day 3, Dynabeads are removed by magnet. T-cells are counted and passaged (0.5×10⁶ cells/mL). During subsequent expansion, cells are passaged every two days (0.5×10⁶ cells/mL).

### Co-Culture Assay

On Day 7, an aliquot of each cell population is stained with PE conjugated anti-MYC and BV421 conjugated anti-FLAG antibodies (corresponding to aCAR and iCAR, respectively), and their transgene expression is quantified using an LX CytoFlex Flow Cytometry machine. On Day 8, T-cells are counted and distributed into a 96-well plate for co-culture assays, with each well containing 5×10⁵ K562 target cells either engineered to co-express aCAR target CD20 and iCAR target CD19 or engineered to express CD20 alone and stained with CellTrace Violet dye (Invitrogen) and 5×10⁵ aCAR+ or dual+ T-cells. Co-cultures are incubated (37°, 5% CO₂) for 40 hrs. On Day 10, cells in co-cultures are stained with NIR viability dye (Biolegend) and the number of live target cells is quantified using a CytoFlex LX flow cytometer. Killing efficiencies for each engineered CAR-T cell population are calculated as the ratio of surviving wild type K562 relative to each of the CD20-expressing K562 target cell lines. Normalized killing efficiencies are calculated as the ratio of CAR-T killing efficiencies for dual (CD20+CD19+) vs single (CD20+ only) antigen target cells.

### Enzymatic Inhibitory Domain Containing CAR Assessment Results

Inhibition of T cell signaling by a CAR containing an enzymatic inhibitory domain (EID) is assessed. The assessment strategy follows that described in Example 1. Engineered T cells expressing an aCAR alone or co-expressing an aCAR and iCAR are assessed for cytotoxicity, cytokine release, expression of activation-associated markers when co-cultured with engineered target cells expressing cognate antigens recognized by the iCAR, aCAR, both, or neither. Exemplary constructs assessed are described in Table B. Also assessed are aCARs targeting tumor-associated antigens in combination with iCARs targeting antigens generally expressed on healthy tissue and/or cells.

Flow-cytometry analysis of engineered T cells demonstrates co-expression of aCAR and iCAR constructs. The various iCAR constructs are then assessed for their ability to inhibit signaling. Results demonstrate CARs containing an enzymatic inhibitory domain are capable of inhibiting cellular signaling of an activating CAR in a ligand-specific manner, including determining those iCAR features (e.g., EIDs, additional domains, domain organizations, etc.) demonstrating the most robust signaling inhibition and/or ligand-specificity.

TABLE B
Candidate iCAR Constructs

| | Signal | Epitope | | hinge/ | TM | | | | |
|---|---|---|---|---|---|---|---|---|---|
| Construct | Sequence | Tag | scFv | linker | Domain | Linker | Intracellular Domain | 2A | Marker |
| SB00628 | CD8 | | aCD19 | CD8 | CD28 | | Csk | | |
| SB00629 | CD8 | | aCD19 | CD8 | CD28 | | Csk deltaSH3 | | |
| SB00655 | CD8 | | aCD19 | CD8 | LAT (1-33) | | Csk deltaSH3 | | |
| SB00656 | CD8 | | aCD19 | CD8 | PAG (1-46) | | Csk deltaSH3 | | |
| SB01012 | mIgK | FLAG | aCD19 | CD8 | CD28 | | Csk deltaSH3 | | |
| SB01016 | mIgK | FLAG | aCD19 | CD8 | LAT (1-33) | | Csk deltaSH3 | | |
| SB01017 | mIgK | FLAG | aCD19 | CD8 | PAG (1-46) | | Csk deltaSH3 | | |
| SB01025 | mIgK | FLAG | aCD19 | CD8 | CD28 | | Csk | T2A | PuroR |
| SB01026 | mIgK | FLAG | aCD19 | CD8 | CD28 | | Csk deltaSH3 | T2A | PuroR |
| SB01030 | mIgK | FLAG | aCD19 | CD8 | LAT (1-33) | | Csk deltaSH3 | T2A | PuroR |
| SB01031 | mIgK | FLAG | aCD19 | CD8 | PAG (1-46) | | Csk deltaSH3 | T2A | PuroR |
| SB01871 | mIgK | FLAG | aCD19 | CD8 | mLAT | (G4S)3 | Csk deltaSH3 | T2A | GFP |
| SB01872 | mIgK | FLAG | aCD19 | CD8 | mLAT | (G4S)3 | SHP-1 | T2A | GFP |
| SB01873 | mIgK | FLAG | aCD19 | CD8 | mLAT | (G4S)3 | SHP-1 PTP domain | T2A | GFP |
| SB01874 | mIgK | FLAG | aCD19 | CD8 | mLAT | (G4S)3 | SHP-2 | T2A | GFP |
| SB01875 | mIgK | FLAG | aCD19 | CD8 | mLAT(ca) | (G4S)3 | SHP-1 | T2A | GFP |
| SB01876 | mIgK | FLAG | aCD19 | CD8 | LAX | (G4S)3 | SHP-1 | T2A | GFP |
| SB01877 | CD8 | FLAG | aCD19 | CD8 | mLAT | (G4S)3 | SHP-1 PTP domain | T2A | GFP |
| SB01878 | mIgK | FLAG | aCD19 | CD8 | CD28 | (G4S)3 | SHP-1 | T2A | GFP |
| SB01879 | mIgK | FLAG | aCD19 | CD8 | CD3z | (G4S)3 | SHP-1 | T2A | GFP |
| SB01881 | mIgK | FLAG | aCD19 | CD8 | CD45 | | CD45 | T2A | GFP |
| SB01884 | mIgK | FLAG | aCD19 | CD8 | CD8 | (G4S)3 | PTEN | T2A | GFP |
| SB01885 | mIgK | FLAG | aCD19 | CD8 | CD8 | (G4S)3 | PTP-MEG1 | T2A | GFP |
| SB01886 | mIgK | FLAG | aCD19 | CD8 | CD8 | (G4S)3 | PTPN22 | T2A | GFP |
| SB02018 | GM-CSF | FLAG | aCD19 | CD8 | LAT(1-33) | (G4S)3 | Csk | T2A | GFP |
| SB02033 | mIgK | FLAG | aCD19 | CD8 | CD8 | (G4S)3 | Zap70, SH2 domains | T2A | GFP |
| | | | | | | (G4S)3 | 1 and 2 only | | |
| SB02034 | mIgK | FLAG | aCD19 | CD8 | CD8 | (G4S)3 | Zap70 delta kinase | T2A | GFP |
| SB02035 | mIgK | FLAG | aCD19 | CD8 | CD8 | (G4S)3 | Zap70 Y492F Y493F | T2A | GFP |
| SB02162 | mIgK | FLAG | aCD19 | CD8 | CD8 | (G4S)3 | SHIP-1 PTP domain | | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

### ADDITIONAL SEQUENCES

Certain additional sequences for vectors, cassettes and protein domains referred to herein are described below and referred to by SEQ ID NO.

TABLE C
Additional Sequences

| Amino Acid Sequence | SEQ ID NO: | Description |
|---|---|---|
| MALPVTALLLPLALLLHAARP | 81 | CD8 signal sequence |
| METDTLLLWVLLLWVPGSTGAGGS | 82 | mIgK signal sequence |
| MLLLVTSLLLCELPHPAFLLIP | 83 | GM-CSF signal sequence |
| EVKLQESGPGLVAPSQSLSVTCTVSGVSLPDYGVSWIR | 84 | aCD19 scFv |
| QPPRKGLEWLGVIWGSETTYYNSALKSRLTIIKDNSKS | | |
| QVFLKMNSLQTDDTAIYYCAKHYYYGGSYAMDYWG | | |
| QGTSVTVSSGGGGSGGGGSGGGGSDIQMTQTTSSLSAS | | |
| LGDRVTISCRASQDISKYLNWYQQKPDGTVKLLIYHTS | | |
| RLHSGVPSRFSGSGSGTDYSLTISNLEQEDIATYFCQQG | | |
| NTLPYTFGGGTKLEIT | | |
| MALPVTALLLPLALLLHAARPQVQLVQSGAEVKKPGA | 85 | aCAR full coding sequence |
| SVKVSCKASGYTFTNYWMHWVRQAPGQGLEWMGFIT | | |
| PTTGYPEYNQKFKDRVTMTADKSTSTAYMELSSLRSE | | |
| DTAVYYCARRKVGKGVYYALDYWGQGTTVTVSSGG | | |
| GGSGGGGSGGGGSDIQMTQSPSSLSASVGDRVTITCRA | | |
| SGNIHNYLAWYQQKPGKVPKLLIYNTKTLADGVPSRFS | | |
| GSGSGTDYTLTISSLQPEDVATYYCQHFWSSPWTFGGG | | |
| TKVEIKEQKLISEEDLNGAATTTPAPRPPTPAPTIALQPL | | |
| SLRPEACRPAAGGAVHTRGLDFACDFWVLVVVGGVL | | |
| ACYSLLVTVAFIIFWVRSKRSRLLHSDYMNMTPRRPGP | | |
| TRKHYQPYAPPRDFAAYRSRVKFSRSADAPAYKQGQN | | |
| QLYNELNLGRREEYDVLDKRRGRDPEMGGKPRRKNP | | |
| QEGLYNELQKDKMAEAYSEIGMKGERRRGKGHDGLY | | |
| QGLSTATKDTYDALHMQALPPR* | | |
| METDTLLLWVLLLWVPGSTGAGGSDYKDDDDKGGSE | 86 | iCAR17 full coding sequence |
| VKLQESGPGLVAPSQSLSVTCTVSGVSLPDYGVSWIRQ | | |
| PPRKGLEWLGVIWGSETTYYNSALKSRLTIIKDNSKSQ | | |
| VFLKMNSLQTDDTAIYYCAKHYYYGGSYAMDYWGQ | | |
| GTSVTVSSGGGGSGGGGSGGGGSDIQMTQTTSSLSASL | | |
| GDRVTISCRASQDISKYLNWYQQKPDGTVKLLIYHTSR | | |
| LHSGVPSRFSGSGSGTDYSLTISNLEQEDIATYFCQQGN | | |
| TLPYTFGGGTKLEITTTTPAPRPPTPAPTIALQPLSLRPE | | |
| ACRPAAGGAVHTRGLDFACDGPAGSLLGSGQMQITLW | | |
| GSLAAVAIFFVITFLIFLCSSCDREKKPRSAIQAAWPSGT | | |
| ECIAKYNFHGTAEQDLPFCKGDVLTIVAVTKDPNWYK | | |
| AKNKVGREGIIPANYVQKREGVKAGTKLSLMPWFHGK | | |
| ITREQAERLLYPPETGLFLVRESTNYPGDYTLCVSCDGK | | |
| VEHYRIMYHASKLSIDEEVYFENLMQLVEHYTSDADG | | |
| LCTRLIKPKVMEGTVAAQDEFYRSGWALNMKELKLLQ | | |
| TIGKGEFGDVMLGDYRGNKVAVKCIKNDATAQAFLAE | | |
| ASVMTQLRHSNLVQLLGVIVEEKGGLYIVTEYMAKGS | | |
| LVDYLRSRGRSVLGGDCLLKFSLDVCEAMEYLEGNNF | | |
| VHRDLAARNVLVSEDNVAKVSDFGLTKEASSTQDTGK | | |
| LPVKWTAPEALREKKFSTKSDVWSFGILLWEIYSFGRV | | |
| PYPRIPLKDVVPRVEKGYKMDAPDGCPPAVYEVMKNC | | |
| WHLDAAMRPSFLQLREQLEHIKTHELHL* | | |
| METDTLLLWVLLLWVPGSTGAGGSDYKDDDDKGGSE | 87 | iCAR25 full coding sequence |
| VKLQESGPGLVAPSQSLSVTCTVSGVSLPDYGVSWIRQ | | |
| PPRKGLEWLGVIWGSETTYYNSALKSRLTIIKDNSKSQ | | |
| VFLKMNSLQTDDTAIYYCAKHYYYGGSYAMDYWGQ | | |
| GTSVTVSSGGGGSGGGGSGGGGSDIQMTQTTSSLSASL | | |
| GDRVTISCRASQDISKYLNWYQQKPDGTVKLLIYHTSR | | |
| LHSGVPSRFSGSGSGTDYSLTISNLEQEDIATYFCQQGN | | |
| TLPYTFGGGTKLEITTTTPAPRPPTPAPTIALQPLSLRPE | | |
| ACRPAAGGAVHTRGLDFACDFWVLVVVGGVLACYSL | | |
| LVTVAFIIFWVSAIQAAWPSGTECIAKYNFHGTAEQDLP | | |
| FCKGDVLTIVAVTKDPNWYKAKNKVGREGIIPANYVQ | | |
| KREGVKAGTKLSLMPWFHGKITREQAERLLYPPETGLF | | |
| LVRESTNYPGDYTLCVSCDGKVEHYRIMYHASKLSIDE | | |
| EVYFENLMQLVEHYTSDADGLCTRLIKPKVMEGTVAA | | |
| QDEFYRSGWALNMKELKLLQTIGKGEFGDVMLGDYR | | |
| GNKVAVKCIKNDATAQAFLAEASVMTQLRHSNLVQLL | | |
| GVIVEEKGGLYIVTEYMAKGSLVDYLRSRGRSVLGGD | | |
| CLLKFSLDVCEAMEYLEGNNFVHRDLAARNVLVSEDN | | |
| VAKVSDFGLTKEASSTQDTGKLPVKWTAPEALREKKF | | |
| STKSDVWSFGILLWEIYSFGRVPYPRIPLKDVVPRVEKG | | |
| YKMDAPDGCPPAVYEVMKNCWHLDAAMRPSFLQLRE | | |
| QLEHIKTHELHLEGRGSLLTCGDVEENPGPMTEYKPTV | | |
| RLATRDDVPRAVRTLAAAFADYPATRHTVDPDRHIER | | |
| VTELQELFLTRVGLDIGKVWVADDGAAVAVWTTPESV | | |
| EAGAVFAEIGPRMAELSGSRLAAQQQMEGLLAPHRPK | | |
| EPAWFLATVGVSPDHQGKGLGSAVVLPGVEAAERAG | | |
| VPAFLETSAPRNLPFYERLGFTVTADVEVPEGPRTWCM | | |
| TRKPGA* | | |
| METDTLLLWVLLLWVPGSTGAGGSDYKDDDDKGGSE | 88 | iCAR26 full coding sequence |
| VKLQESGPGLVAPSQSLSVTCTVSGVSLPDYGVSWIRQ | | |
| PPRKGLEWLGVIWGSETTYYNSALKSRLTIIKDNSKSQ | | |
| VFLKMNSLQTDDTAIYYCAKHYYYGGSYAMDYWGQ | | |
| GTSVTVSSGGGGSGGGGSGGGGSDIQMTQTTSSLSASL | | |
| GDRVTISCRASQDISKYLNWYQQKPDGTVKLLIYHTSR | | |
| LHSGVPSRFSGSGSGTDYSLTISNLEQEDIATYFCQQGN | | |
| TLPYTFGGGTKLEITTTTPAPRPPTPAPTIALQPLSLRPE | | |
| ACRPAAGGAVHTRGLDFACDFWVLVVVGGVLACYSL | | |
| LVTVAFIIFWVVKAGTKLSLMPWFHGKITREQAERLLY | | |
| PPETGLFLVRESTNYPGDYTLCVSCDGKVEHYRIMYHA | | |
| SKLSIDEEVYFENLMQLVEHYTSDADGLCTRLIKPKVM | | |
| EGTVAAQDEFYRSGWALNMKELKLLQTIGKGEFGDV | | |
| MLGDYRGNKVAVKCIKNDATAQAFLAEASVMTQLRH | | |
| SNLVQLLGVIVEEKGGLYIVTEYMAKGSLVDYLRSRGR | | |
| SVLGGDCLLKFSLDVCEAMEYLEGNNFVHRDLAARNV | | |
| LVSEDNVAKVSDFGLTKEASSTQDTGKLPVKWTAPEA | | |
| LREKKFSTKSDVWSFGILLWEIYSFGRVPYPRIPLKDVV | | |
| PRVEKGYKMDAPDGCPPAVYEVMKNCWHLDAAMRP | | |
| SFLQLREQLEHIKTHELHLEGRGSLLTCGDVEENPGPM | | |
| TEYKPTVRLATRDDVPRAVRTLAAAFADYPATRHTVD | | |
| PDRHIERVTELQELFLTRVGLDIGKVWVADDGAAVAV | | |
| WTTPESVEAGAVFAEIGPRMAELSGSRLAAQQQMEGL | | |
| LAPHRPKEPAWFLATVGVSPDHQGKGLGSAVVLPGVE | | |
| AAERAGVPAFLETSAPRNLPFYERLGFTVTADVEVPEG | | |
| PRTWCMTRKPGA* | | |
| METDTLLLWVLLLWVPGSTGAGGSDYKDDDDKGGSE | 89 | iCAR30 full coding sequence |
| VKLQESGPGLVAPSQSLSVTCTVSGVSLPDYGVSWIRQ | | |
| PPRKGLEWLGVIWGSETTYYNSALKSRLTIIKDNSKSQ | | |
| VFLKMNSLQTDDTAIYYCAKHYYYGGSYAMDYWGQ | | |
| GTSVTVSSGGGGSGGGGSGGGGSDIQMTQTTSSLSASL | | |
| GDRVTISCRASQDISKYLNWYQQKPDGTVKLLIYHTSR | | |
| LHSGVPSRFSGSGSGTDYSLTISNLEQEDIATYFCQQGN | | |
| TLPYTFGGGTKLEITTTTPAPRPPTPAPTIALQPLSLRPE | | |
| ACRPAAGGAVHTRGLDFACDEEAILVPCVLGLLLLPIL | | |
| AMLMALCVHCHRLPSAIQAAWPSGTECIAKYNFHGTA | | |
| EQDLPFCKGDVLTIVAVTKDPNWYKAKNKVGREGIIPA | | |
| NYVQKREGVKAGTKLSLMPWFHGKITREQAERLLYPP | | |
| ETGLFLVRESTNYPGDYTLCVSCDGKVEHYRIMYHAS | | |
| KLSIDEEVYFENLMQLVEHYTSDADGLCTRLIKPKVME | | |
| GTVAAQDEFYRSGWALNMKELKLLQTIGKGEFGDVM | | |
| LGDYRGNKVAVKCIKNDATAQAFLAEASVMTQLRHS | | |
| NLVQLLGVIVEEKGGLYIVTEYMAKGSLVDYLRSRGRS | | |
| VLGGDCLLKFSLDVCEAMEYLEGNNFVHRDLAARNVL | | |
| VSEDNVAKVSDFGLTKEASSTQDTGKLPVKWTAPEAL | | |
| REKKFSTKSDVWSFGILLWEIYSFGRVPYPRIPLKDVVP | | |
| RVEKGYKMDAPDGCPPAVYEVMKNCWHLDAAMRPS | | |
| FLQLREQLEHIKTHELHLEGRGSLLTCGDVEENPGPMT | | |
| EYKPTVRLATRDDVPRAVRTLAAAFADYPATRHTVDP | | |
| DRHIERVTELQELFLTRVGLDIGKVWVADDGAAVAVW | | |
| TTPESVEAGAVFAEIGPRMAELSGSRLAAQQQMEGLLA | | |
| PHRPKEPAWFLATVGVSPDHQGKGLGSAVVLPGVEAA | | |
| ERAGVPAFLETSAPRNLPFYERLGFTVTADVEVPEGPR | | |
| TWCMTRKPGA* | | |
| METDTLLLWVLLLWVPGSTGAGGSDYKDDDDKGGSE | 90 | iCAR31 full coding sequence |
| VKLQESGPGLVAPSQSLSVTCTVSGVSLPDYGVSWIRQ | | |
| PPRKGLEWLGVIWGSETTYYNSALKSRLTIIKDNSKSQ | | |
| VFLKMNSLQTDDTAIYYCAKHYYYGGSYAMDYWGQ | | |
| GTSVTVSSGGGGSGGGGSGGGGSDIQMTQTTSSLSASL | | |
| GDRVTISCRASQDISKYLNWYQQKPDGTVKLLIYHTSR | | |
| LHSGVPSRFSGSGSGTDYSLTISNLEQEDIATYFCQQGN | | |
| TLPYTFGGGTKLEITTTTPAPRPPTPAPTIALQPLSLRPE | | |
| ACRPAAGGAVHTRGLDFACDGPAGSLLGSGQMQITLW | | |
| GSLAAVAIFFVITFLIFLCSSCDREKKPRSAIQAAWPSGT | | |
| ECIAKYNFHGTAEQDLPFCKGDVLTIVAVTKDPNWYK | | |
| AKNKVGREGIIPANYVQKREGVKAGTKLSLMPWFHGK | | |
| ITREQAERLLYPPETGLFLVRESTNYPGDYTLCVSCDGK | | |
| VEHYRIMYHASKLSIDEEVYFENLMQLVEHYTSDADG | | |
| LCTRLIKPKVMEGTVAAQDEFYRSGWALNMKELKLLQ | | |
| TIGKGEFGDVMLGDYRGNKVAVKCIKNDATAQAFLAE | | |
| ASVMTQLRHSNLVQLLGVIVEEKGGLYIVTEYMAKGS | | |
| LVDYLRSRGRSVLGGDCLLKFSLDVCEAMEYLEGNNF | | |
| VHRDLAARNVLVSEDNVAKVSDFGLTKEASSTQDTGK | | |
| LPVKWTAPEALREKKFSTKSDVWSFGILLWEIYSFGRV | | |
| PYPRIPLKDVVPRVEKGYKMDAPDGCPPAVYEVMKNC | | |
| WHLDAAMRPSFLQLREQLEHIKTHELHLEGRGSLLTCG | | |
| DVEENPGPMTEYKPTVRLATRDDVPRAVRTLAAAFAD | | |
| YPATRHTVDPDRHIERVTELQELFLTRVGLDIGKVWVA | | |
| DDGAAVAVWTTPESVEAGAVFAEIGPRMAELSGSRLA | | |
| AQQQMEGLLAPHRPKEPAWFLATVGVSPDHQGKGLG | | |
| SAVVLPGVEAAERAGVPAFLETSAPRNLPFYERLGFTV | | |
| TADVEVPEGPRTWCMTRKPGA* | | |

What is claimed is:

1. A chimeric inhibitory receptor comprising:
an extracellular ligand binding domain;
a membrane localization domain, wherein the membrane localization domain comprises a transmembrane domain; and
an enzymatic inhibitory domain, wherein the enzymatic inhibitory domain inhibits immune receptor activation when proximal to an immune receptor.

2. The chimeric inhibitory receptor of claim 1, wherein the enzymatic inhibitory domain comprises an enzyme catalytic domain, and wherein the enzyme catalytic domain is from an enzyme selected from the group consisting of: CSK, SHP-1, SHP-2, PTEN, CD45, CD148, PTP-MEG1, PTP-PEST, c-CBL, CBL-b, PTPN22, LAR, PTPH1, SHIP-1, ZAP70, and RasGAP.

3. The chimeric inhibitory receptor of claim 1 or claim 2, wherein the enzymatic inhibitory domain comprises at least a portion of an extracellular domain, a transmembrane domain, and/or an intracellular domain.

4. The chimeric inhibitory receptor of any one of claims 1-3, wherein the extracellular ligand binding domain comprises an antibody, or antigen-binding fragment thereof, optionally wherein the or antigen-binding fragment is a a F(ab) fragment, a F(ab′) fragment, or a single chain variable fragment (scFv).

5. The chimeric inhibitory receptor of any one of claims 1-4, wherein the extracellular ligand binding domain binds to a ligand that is not expressed on a tumor cell and/or the ligand is expressed on cells of a healthy tissue.

6. The chimeric inhibitory receptor of any one of claims 1-5, wherein the extracellular ligand binding domain comprises a dimerization domain, optionally wherein the ligand further comprises a cognate dimerization domain.

7. The chimeric inhibitory receptor of any one of claims 1-6, wherein the membrane localization domain further comprises at least a portion of an extracellular domain and/or at least a portion of an intracellular domain, and optionally wherein the transmembrane domain is selected from the group consisting of: a LAX transmembrane domain, a CD25 transmembrane domain, a CD7 transmembrane domain, a LAT transmembrane domain, a transmembrane domain from a LAT mutant, a BTLA transmembrane domain, a CD8 transmembrane domain, a CD28 transmembrane domain, a CD3zeta transmembrane domain, a CD4 transmembrane domain, a 4-IBB transmembrane domain, an OX40 transmembrane domain, an ICOS transmembrane domain, a 2B4 transmembrane domain, a PD-1 transmembrane domain, a CTLA4 transmembrane domain, a BTLA transmembrane domain, a TIM3 transmembrane domain, a LIR1 transmembrane domain, an NKG2A transmembrane domain, a TIGIT transmembrane domain, and a LAG3 transmembrane domain, a LAIR1 transmembrane domain, a GRB-2 transmembrane domain, a Dok-1 transmembrane domain, a Dok-2 transmembrane domain, a SLAP1 transmembrane domain, a SLAP2 transmembrane domain, a CD200R transmembrane domain, an SIRPa transmembrane domain, an HAVR transmembrane domain, a GITR transmembrane domain, a PD-L1 transmembrane domain, a KIR2DL1 transmembrane domain, a KIR2DL2 transmembrane domain, a KIR2DL3 transmembrane domain, a KIR3DL1 transmembrane domain, a KIR3DL2 transmembrane domain, a CD94 transmembrane domain, a KLRG-1 transmembrane domain, a PAG transmembrane domain, a CD45 transmembrane domain, and a CEACAM1 transmembrane domain.

8. The chimeric inhibitory receptor of any one of claims 1-7, wherein the chimeric inhibitory receptor further comprises one or more intracellular inhibitory co-signaling domains, optionally wherein the one or more intracellular inhibitory co-signaling domains comprise one or more ITIM-containing proteins, or fragments thereof, selected from the group consisting of: PD-1, CTLA4, TIGIT, BTLA, and LAIR1; and/or the one or more intracellular inhibitory co-signaling domains comprise one or more non-ITIM scaffold proteins, or fragments thereof, selected from the group consisting of: GRB-2, Dok-1, Dok-2, SLAP1, SLAP2, LAG3, HAVR, GITR, and PD-L1.

9. The chimeric inhibitory receptor of any one of claims 1-8, wherein the extracellular ligand binding domain is linked to the membrane localization domain through an extracellular linker region, optionally wherein the extracellular linker region is positioned between the extracellular ligand binding domain and membrane localization domain and operably and/or physically linked to each of the extracellular ligand binding domain and the membrane localization domain, optionally wherein the extracellular linker region is derived from a protein selected from the group consisting of: CD8alpha, CD4, CD7, CD28, IgG1, IgG4, FcgammaRIIIalpha, LNGFR, and PDGFR or comprises an amino acid sequence selected from the group consisting of:
(SEQ ID NO: 29)
GGS,
(SEQ ID NO: 30)
GGSGGS,
(SEQ ID NO: 31)
GGSGGSGGS,
(SEQ ID NO: 32)
GGSGGSGGSGGS,
(SEQ ID NO: 33)
GGSGGSGGSGGSGGS,
(SEQ ID NO: 34)
GGGS,
(SEQ ID NO: 35)
GGGSGGGS,
(SEQ ID NO: 36)
GGGSGGGSGGGS,
(SEQ ID NO: 37)
GGGSGGGSGGGSGGGS,
(SEQ ID NO: 38)
GGGSGGGSGGGSGGGSGGGS,
(SEQ ID NO: 39)
GGGGS,
(SEQ ID NO: 40)
GGGGSGGGGS,
(SEQ ID NO: 41)
GGGGSGGGGSGGGGS,
(SEQ ID NO: 42)
GGGGSGGGGSGGGGSGGGGS,
(SEQ ID NO: 43)
GGGGSGGGGSGGGGSGGGGSGGGGS,
(SEQ ID NO: 46)
AAAIEVMYPPPYLDNEKSNGTIIHVKGKHLCPSPLFPGPSKP,
(SEQ ID NO: 47)
ESKYGPPCPSCP,
(SEQ ID NO: 48)
ESKYGPPAPSAP,
(SEQ ID NO: 49)
ESKYGPPCPPCP,
(SEQ ID NO: 50)
EPKSCDKTHTCP,
(SEQ ID NO: 51)
AAAFVPVFLPAKPTTTPAPRPPTPAPTIASQPLSLRPEACRPAAGGAVHT
RGLDFACDIYIWAPLAGTCGVLLLSLVITLYCNHRN,
(SEQ ID NO: 52)
TTTPAPRPPTPAPTIALQPLSLRPEACRPAAGGAVHTRGLDFACD,
(SEQ ID NO: 53)
ACPTGLYTHSGECCKACNLGEGVAQPCGANQTVCEPCLDSVTFSDVVSAT
EPCKPCTECVGLQSMSAPCVEADDAVCRCAYGYYQDETTGRCEACRVCEA
GSGLVFSCQDKQNTVCEECPDGTYSDEADAEC,
(SEQ ID NO: 54)
ACPTGLYTHSGECCKACNLGEGVAQPCGANQTVC,
and
(SEQ ID NO: 55)
AVGQDTQEVIVVPHSLPFKV.

10. The chimeric inhibitory receptor of any one of claims 1-9, wherein the chimeric inhibitory receptor further comprises an intracellular spacer region positioned between the membrane localization domain and the enzymatic inhibitory domain and operably and/or physically linked to each of the membrane localization domain and the enzymatic inhibitory domain, optionally, wherein the intracellular spacer region comprises an amino acid sequence selected from the group consisting of: GGS (SEQ ID NO: 29), GGSGGS (SEQ ID NO: 30), GGSGGSGGS (SEQ ID NO: 31), GGSGGSGGSGGS (SEQ ID NO: 32), GGSGGSGGSGGSGGS (SEQ ID NO: 33), GGGS (SEQ ID NO: 34), GGGSGGGS (SEQ ID NO: 35), GGGSGGGSGGGS (SEQ ID NO: 36), GGGSGGGSGGGSGGGS (SEQ ID NO: 37), GGGSGGGSGGGSGGGSGGGS (SEQ ID NO: 38), GGGGS (SEQ ID NO: 39), GGGGSGGGGS (SEQ ID NO: 40), GGGGSGGGGSGGGGS (SEQ ID NO: 41), GGGGSGGGGSGGGGSGGGGS (SEQ ID NO: 42), GGGGSGGGGSGGGGSGGGGSGGGGS (SEQ ID NO: 43), AAAIEVMYPPPYLDNEKSNGTIIHVKGKHLCPSPLFPGPSKP (SEQ ID NO:46), ESKYGPPCPSCP (SEQ ID NO:47), ESKYGPPAPSAP (SEQ ID NO:48), ESKYGPPCPPCP (SEQ ID NO:49), EPKSCDKTHTCP (SEQ ID NO:50), AAAFVPVFLPAKPTTTPAPRPPTPAPTIASQPLSLRPEACRPAAGGAVHTRGLD FACDIYIWAPLAGTCGVLLLSLVITLYCNHRN (SEQ ID NO:51), TTTPAPRPPTPAPTIALQPLSLRPEACRPAAGGAVHTRGLDFACD (SEQ ID NO:52), ACPTGLYTHSGECCKACNLGEGVAQPCGANQTVCEPCLDSVTFSDVVSATEP CKPCTECVGLQSMSAPCVEADDAVCRCAYGYYQDETTGRCEACRVCEAGSG LVFSCQDKQNTVCEECPDGTYSDEADAEC (SEQ ID NO:53), ACPTGLYTHSGECCKACNLGEGVAQPCGANQTVC (SEQ ID NO:54), and AVGQDTQEVIVVPHSLPFKV (SEQ ID NO:55).

11. The chimeric inhibitory receptor of any one of claims 1-10, wherein the immune receptor is a chimeric immune receptor, optionally wherein the immune receptor is a chimeric antigen receptor (CAR), a naturally-occurring antigen receptor, optionally wherein the immune receptor is selected from the group consisting of a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor.

12. A nucleic acid encoding the chimeric inhibitory receptor of any one of claims 1-11.

13. A vector comprising the nucleic acid of claim 12.

14. A genetically engineered cell comprising the nucleic acid of claim 12, the vector of claim 13 or expressing the chimeric inhibitory receptor of any one of claims 1-11.

15. A genetically engineered cell expressing a chimeric inhibitory receptor, wherein the chimeric inhibitory receptor comprises:
an extracellular ligand binding domain;
a membrane localization domain, wherein the membrane localization domain comprises a transmembrane domain; and
an enzymatic inhibitory domain, wherein the inhibitory domain inhibits immune receptor activation when proximal to an immune receptor,
optionally wherein the cell further comprises an immune receptor,
optionally wherein the immune receptor is a chimeric antigen receptor or a naturally-occurring antigen receptor,
optionally wherein the immune receptor is selected from the group consisting of: a T cell receptor, a pattern recognition receptor (PRR), a NOD-like receptor (NLR), a Toll-like receptor (TLR), a killer activated receptor (KAR), a killer inhibitor receptor (KIR), a complement receptor, an Fc receptor, a B cell receptor, and a cytokine receptor,
optionally wherein the chimeric inhibitory receptor inhibits immune receptor activation upon ligand binding.

16. The engineered cell of claim 14 or claim 15, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an ESC-derived cell, and an iPSC-derived cell.

17. A pharmaceutical composition comprising the engineered cell of any one of claims 14-16 and a pharmaceutically acceptable carrier, a pharmaceutically acceptable excipient, or combination thereof.

18. A method of inhibiting immune receptor activation, comprising:
contacting the engineered cell of any one of claims 14-16 or the pharmaceutical composition of claim 17 with a cognate ligand under conditions suitable for the chimeric inhibitory receptor to bind the cognate ligand,
wherein, when localized proximal to an immune receptor expressed on a cell membrane of the engineered cell, the chimeric inhibitory inhibits immune receptor activation.

19. A method of preventing, attenuating, or inhibiting a cell-mediated immune response induced by a tumor-targeting chimeric receptor expressed on the surface of an immunomodulatory cell, comprising:
administering the engineered cell of any one of claims 14-16 or the pharmaceutical composition of claim 17 to a subject in need of such treatment.

20. A method of preventing, attenuating, or inhibiting activation of a tumor-targeting chimeric receptor expressed on the surface of an immunomodulatory cell, comprising:
contacting the engineered cell of any one claims 14-16 or the pharmaceutical composition of claim 17 or the pharmaceutical composition of claim 17 with a cognate ligand of the chimeric inhibitory receptor under conditions suitable for the chimeric inhibitory receptor to bind the cognate ligand,
wherein upon binding of the ligand to the chimeric inhibitory receptor, the enzymatic inhibitory domain prevents, attenuates, or inhibits activation of the tumor-targeting chimeric receptor.


SEQUENCE LISTING (90 sequences)

SEQ ID NO: 1 - protein, 449 aa, Artificial Sequence
  Description of Artificial Sequence Synthetic polypeptide
SAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 2 - protein, 380 aa, Artificial Sequence
  Description of Artificial Sequence Synthetic polypeptide
VKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 3 - protein, 595 aa, Artificial Sequence
  Description of Artificial Sequence Synthetic polypeptide
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 4 - protein, 272 aa, Artificial Sequence
  Description of Artificial Sequence Synthetic polypeptide
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 5 - protein, 597 aa, Artificial Sequence
  Description of Artificial Sequence Synthetic polypeptide
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 6 - protein, 708 aa, Artificial Sequence
  Description of Artificial Sequence Synthetic polypeptide
YKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 7 - protein, 217 aa, Artificial Sequence [sequence omitted]

SEQ ID NO: 8 - protein, 403 aa, Artificial Sequence [sequence omitted]

SEQ ID NO: 9 - protein, 926 aa, Artificial Sequence
  Description of Artificial Sequence Synthetic polypeptide
MTSRFRLPAGRTYNVRASELARDRQHTEVVCNILLLDNTVQAFKVNKHDQGQVLLDVVFKHLDLTEQDYFGLQLADDSTDNPRWLDPNKPIRKQLKRGSPYSLNFRVKFFVSDPNKLQEEYTRYQYFLQIKQDILTGRLPCPSNTAALLASFAVQSELGDYDQSENLSGYLSDYSFIPNQPQDFEKEIAKLHQQHIGLSPAEAEFNYLNTARTLELYGVEFHYARDQSNNEIMIGVMSGGILIYKNRVRMNTFPWLKIVKISFKCKQFFIQLRKELHESRETLLGFNMVNYRACKNLWKACVEHHTFFRLDRPLPPQKNFFAHYFTLGSKFRYCGRTEVQSVQYGKEKANKDRVFARSPSKPLARKLMDWEVVSRNSISDDRLETQSLPSRSPPGTPNHRNSTFTQEGTRLRPSSVGHLVDHMVHTSPSEVFVNQRSPSSTQANSIVLESSPSQETPGDGKPPALPPKQSKKNSWNQIHYSHSQQDLESHINETFDIPSSPEKPTPNGGIPHDNLVLIRMKPDENGRFGFNVKGGYDQKMPVIVSRVAPGTPADLCVPRLNEGDQVVLINGRDIAEHTHDQVVLFIKASCERHSGELMLLVRPNAVYDVVEEKLENEPDFQYIPEKAPLDSVHQDDHSLRESMIQLAEGLITGTVLTQFDQLYRKKPGMTMSCAKLPQNISKNRYRDISPYDATRVILKGNEDYINANYINMEIPSSSIINQYIACQGPLPHTCTDFWQMTWEQGSSMVVMLTTQVERGRVKCHQYWPEPTGSSSYGCYQVTCHSEEGNTAYIFRKMTLFNQEKNESRPLTQIQYIAWPDHGVPDDSSDFLDFVCHVRNKRAGKEEPVVVHCSAGIGRTGVLITMETAMCLIECNQPVYPLDIVRTMRDQRAMMIQTPSQYRFVCEAILKVYEEGFVKPLTTSTNK

SEQ ID NO: 10 - protein, 806 aa, Artificial Sequence
  Description of Artificial Sequence Synthetic polypeptide
DQREILQKFLDEAQSKKITKEEFANEFLKLKRQSTKYKADKTYPTTVAEKPKNIKKNRYKDILPYDYSRVELSLITSDEDSSYINANFIKGVYGPKAYIATQGPLSTTLLDFWRMIWEYSVLIIVMACMEYEMGKKKCERYWAEPGEMQLEFGPFSVSCEAEKRKSDYIIRTLKVKFNSETRTIYQFHYKNWPDHDVPSSIDPILELIWDVRCYQEDDSVPICIHCSAGCGRTGVICAIDYTWMLLKDGIIPENFSVFSLIREMRTQRPSLVQTQEQYELVYNAVLELFKRQMDVIRDKHSGTESQAKHCIPEKNHTLQADSYSPNLPKSTTKAAKMMNQQRTKMEIKESSSFDFRTSEISAKEELVLHPAKSSTSFDFLELNYSFDKNADTTMKWQTKAFPIVGEPLQKHQSLDLGSLLFEGCSNSKPVNAAGRYFNSKVPITRTKSTPFELIQQRETKEVDSKENFSYLESQPHDSCFVEMQAQKVMHVSSAELNYSLPYDSKHQIRNASNVKHHDSSALGVYSYIPLVENPYFSSWPPSGTSSKMSLDLPEKQDGTVFPSSLLPTSSTSLFSYYNSHDSLSLNSPTNISSLLNQESAVLATAPRIDDEIPPPLPVRTPESFIVVEEAGEFSPNVPKSLSSAVKVKIGTSLEWGGTSEPKKFDDSVILRPSKSVKLRSPKSELHQDRSSPPPPLPERTLESFFLADEDCMQAQSIETYSTSYPDTMENSTSSKQTLKTPGKSFTRSKSLKILRNMKKSICNSCPPNKPAESVQSNNSSSFLNFGFANRFSKPKGPRNPPPTWNI

SEQ ID NO: 11 - protein, 254 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 12 - protein, 337 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNL

SEQ ID NO: 13 - protein, 619 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSFFTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 14 - protein, 317 aa, Artificial Sequence
  Description of Artificial Sequence Synthetic polypeptide
DMITIFIGTWNMGNAPPPKKITSWFLSKGQGKTRDDSADYIPHDIYVIGTQEDPLSEKEWLEILKHSLQEITSVTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWFGDLNYRVDLPTWEAETIIQKIKQQQYADLLSHDQLLTERREQKVFLHFEEEEITFAPTYRFERLTRDKYAYTKQKATGMKYNLPSWCDRVLWKSYPLVHVVCQSYGSTSDIMTSDHSPVFATFEAGVTSQFVS

SEQ ID NO: 15 - DNA, 1347 bp, Artificial Sequence
  Description of Artificial Sequence Synthetic polynucleotide
agtgccattcaggctgcatggccgtcaggtacggagtgtattgctaaatacaattttcacgggactgctgaacaggaccttcctttttgcaaaggggacgtcttgactatcgtagctgtgacgaaagacccgaactggtacaaagctaagaataaggtcggccgggagggtataattcccgcaaattacgtgcagaagcgagaaggtgttaaagctggaacaaagttgtcactcatgccgtggtttcatggaaaaattaccagagaacaagcggagcggctgttgtacccgccggaaaccggccttttcttggttagggaaagcaccaattaccctggggactacactctttgtgtttcatgcgatgggaaggtagaacattaccgcatcatgtatcatgccagtaagctttccatagacgaagaggtgtacttcgagaacctgatgcaactggtggagcactacacatccgatgcggacggattgtgcacgcgattgataaaaccaaaggtaatggaaggcaccgtggcagctcaggatgagttttaccgaagcggttgggcactgaacatgaaagaacttaaactgcttcagacaattgggaaaggagaatttggcgacgtgatgctgggtgattataggggtaacaaggtcgcggtgaagtgcattaagaacgatgccacggcccaggcctttctggcagaagcttcagttatgactcaactccgccattctaatctggtccaattgcttggagtgatcgtcgaagagaagggaggcctctacatagtaacggagtatatggctaaaggttctctcgtcgattatctccgctcacgcgggcgcagcgttctcgggggagattgcctcctgaaattcagtctggacgtctgcgaggctatggagtacctcgaaggaaacaattttgtccaccgggacttggctgcaaggaacgtcctggtaagcgaagataacgtggctaaggtgtccgacttcgggctcacgaaagaagcaagtagtactcaagacacgggcaaactcccagttaaatggaccgcaccggaggcactcagggaaaaaaaattttctaccaagagtgacgtatggtcattcggcatcctcttgtgggaaatatatagttttgggcgcgttccttacccaagaatccccctgaaggatgtcgtgccgagagtggaaaaggggtacaagatggatgctccggacggatgccccccagcagtatatgaggtaatgaaaaattgctggcatctcgatgcagcgatgcggccgtcctttcttcagctccgggagcaactggaacacataaagacgcacgaacttcatctt

SEQ ID NO: 16 - DNA, 1140 bp, Artificial Sequence
  Description of Artificial Sequence Synthetic polynucleotide
gttaaagctggaacaaagttgtcactcatgccgtggtttcatggaaaaattaccagagaacaagcggagcggctgttgtacccgccggaaaccggccttttcttggttagggaaagcaccaattaccctggggactacactctttgtgtttcatgcgatgggaaggtagaacattaccgcatcatgtatcatgccagtaagctttccatagacgaagaggtgtacttcgagaacctgatgcaactggtggagcactacacatccgatgcggacggattgtgcacgcgattgataaaaccaaaggtaatggaaggcaccgtggcagctcaggatgagttttaccgaagcggttgggcactgaacatgaaagaacttaaactgcttcagacaattgggaaaggagaatttggcgacgtgatgctgggtgattataggggtaacaaggtcgcggtgaagtgcattaagaacgatgccacggcccaggcctttctggcagaagcttcagttatgactcaactccgccattctaatctggtccaattgcttggagtgatcgtcgaagagaagggaggcctctacatagtaacggagtatatggctaaaggttctctcgtcgattatctccgctcacgcgggcgcagcgttctcgggggagattgcctcctgaaattcagtctggacgtctgcgaggctatggagtacctcgaaggaaacaattttgtccaccgggacttggctgcaaggaacgtcctggtaagcgaagataacgtggctaaggtgtccgacttcgggctcacgaaagaagcaagtagtactcaagacacgggcaaactcccagttaaatggaccgcaccggaggcactcagggaaaaaaaattttctaccaagagtgacgtatggtcattcggcatcctcttgtgggaaatatatagttttgggcgcgttccttacccaagaatccccctgaaggatgtcgtgccgagagtggaaaaggggtacaagatggatgctccggacggatgccccccagcagtatatgaggtaatgaaaaattgctggcatctcgatgcagcgatgcggccgtcctttcttcagctccgggagcaactggaacacataaagacgcacgaacttcatctt

SEQ ID NO: 17 - DNA, 1785 bp, Artificial Sequence
  Description of Artificial Sequence Synthetic polynucleotide
atggttcgatggttccacagagatctgagcggcctggatgccgagactctgcttaaaggacggggcgtgcacggcagctttctggctagacccagcagaaagaaccagggcgacttcagcctgtccgtcagagtgggcgatcaagtgacacacatcagaatccagaactccggcgacttctacgacctgtacggcggcgagaagttcgccacactgacagagctggtggaatattacacccagcagcaaggcgtgctgcaggacagagatggcaccatcatccacctgaagtaccctctgaactgcagcgaccccaccagcgagagatggtatcacggacacatgtctggcggccaggctgagacactgcttcaggctaaaggcgagccctggacctttctcgtgcgggaatctctgagtcagcccggcgattttgtgctgagcgtgctgtccgatcagcccaaagctggaccaggctctccactgagagtgacccatatcaaagtgatgtgcgaaggcggacggtacaccgtcggtggcctggaaacattcgatagcctgaccgacctggtcgagcacttcaagaaaaccggcatcgaggaagccagcggcgccttcgtttatctgagacagccctactacgccacaagagtgaacgccgccgacatcgagaacagagtgctggaactgaacaagaagcaagagagcgaggataccgccaaggccggcttctgggaagagttcgagtccctgcagaaacaagaagtgaagaacctgcaccagcggctggaaggacagaggcctgagaacaagggcaagaaccggtacaagaacatcctgccattcgaccactccagagtgatcctgcagggaagagacagcaacatccccggctccgactacatcaacgccaattacatcaagaaccagctgctgggccccgacgagaacgccaagacatatatcgcctctcagggctgcctggaagccaccgtgaacgacttttggcagatggcctggcaagagaacagccgcgtgatcgtgatgaccaccagagaggtggaaaaaggccggaacaaatgcgtgccctactggcccgaagtgggcatgcaaagagcctacggaccttacagcgtgaccaactgcggcgagcacgataccaccgagtacaagctgagaaccctccaggtgtcccctctggacaacggcgacctgatcagagagatctggcactaccagtacctgtcctggcctgatcacggcgtgccatctgaacctggtggcgtgctgagtttcctggaccagatcaaccagagacaagagagcctgcctcacgctggccctatcatcgtgcactgttctgccggcatcggcagaaccggcacaatcatcgtgatcgacatgctgatggaaaacatcagcaccaagggcctcgactgcgacatcgacatccagaaaaccatccagatggttcgagcccagcggagcggaatggtgcagacagaagcccagtacaagttcatctacgtggcaatcgcccagttcatcgaaaccaccaagaaaaagctggaagtgctgcagtcccagaagggccaagaaagcgagtacggcaacatcacataccctccagccatgaagaacgcccacgccaaagccagcagaacctccagcaagcacaaagaggacgtctacgagaatctgcacacaaagaacaagcgcgaggaaaaagtgaaaaagcagcgcagcgccgacaaagagaagtccaagggcagcctgaagagaaag

SEQ ID NO: 18 - DNA, 816 bp, Artificial Sequence
  Description of Artificial Sequence Synthetic polynucleotide
ttctgggaagagttcgagtccctgcagaaacaagaagtgaagaacctgcaccagcggctggaaggacagaggcctgagaacaagggcaagaaccggtacaagaacatcctgccattcgaccactccagagtgatcctgcagggaagagacagcaacatccccggctccgactacatcaacgccaattacatcaagaaccagctgctgggccccgacgagaacgccaagacatatatcgcctctcagggctgcctggaagccaccgtgaacgacttttggcagatggcctggcaagagaacagccgcgtgatcgtgatgaccaccagagaggtggaaaaaggccggaacaaatgcgtgccctactggcccgaagtgggcatgcaaagagcctacggaccttacagcgtgaccaactgcggcgagcacgataccaccgagtacaagctgagaaccctccaggtgtcccctctggacaacggcgacctgatcagagagatctggcactaccagtacctgtcctggcctgatcacggcgtgccatctgaacctggtggcgtgctgagtttcctggaccagatcaaccagagacaagagagcctgcctcacgctggccctatcatcgtgcactgttctgccggcatcggcagaaccggcacaatcatcgtgatcgacatgctgatggaaaacatcagcaccaagggcctcgactgcgacatcgacatccagaaaaccatccagatggttcgagcccagcggagcggaatggtgcagacagaagcccagtacaagttcatctacgtggcaatcgcccagttc

SEQ ID NO: 19 - DNA, 1791 bp, Artificial Sequence
  Description of Artificial Sequence Synthetic polynucleotide
atgacaagcagacggtggtttcaccccaacatcaccggcgtggaagctgagaatctgctgctgacaagaggcgtggacggcagctttctggctagacccagcaagtccaatcctggcgacttcacactgagcgtgcggagaaatggcgccgtgacacacatcaagatccagaacaccggcgactactacgacctgtacggcggcgagaagtttgccacactggcagagctggtgcagtactacatggaacaccacggccagctgaaagaaaagaacggcgacgtgatcgagctgaagtaccctctgaactgcgccgatcctaccagcgagagatggtttcacggccacctgtctggcaaagaggccgagaagctgctgaccgagaagggcaagcacggaagctttctcgtgcgcgagtctcagtctcaccccggcgattttgtgctgtctgtgcggacaggggacgacaagggcgagagcaatgacggcaagagcaaagtgacccacgtgatgatccggtgccaagagctgaaatacgacgtcggcggaggggagagattcgactctctgaccgatctggtggaacactacaagaaaaaccccatggtggaaaccctgggcaccgtgctgcagctgaagcagccactgaacaccaccagaatcaacgccgccgagatcgagagcagagtgcgggaactgtctaagctggccgagactaccgacaaagtgaagcaaggcttctgggaagagttcgagacactgcagcagcaagagtgcaagctgctgtactcccggaaagagggccagagacaagagaacaagaacaaaaaccggtacaagaacatcctgccgttcgatcacaccagagtggtgctgcacgacggcgatcctaatgagcccgtgtccgactacatcaacgccaacatcatcatgcccgagtttgagacaaagtgcaacaatagcaagcccaagaagtcctatatcgccacacagggctgcctgcagaataccgtgaacgacttttggcggatggtgtttcaagagaactcccgcgtgatcgtgatgaccaccaaagaggtggaacggggcaagtctaagtgcgtgaagtactggcccgacgagtacgccctgaaagaatacggcgtgatgagagtgcggaacgtgaaagagagcgccgctcacgattacaccctgagagagctgaagctgagcaaagtcggacaggccctgctgcagggaaacaccgaaagaaccgtgtggcagtaccacttccggacctggccagatcatggcgtgccatctgatcctggcggcgtgctggatttcctggaagaggtgcaccacaagcaagagtccatcatggacgccggaccagtggtggtgcactgttctgccggaatcggaagaaccggcaccttcatcgtgatcgacatcctgattgacatcatccgcgagaaaggcgtcgactgcgatatcgacgtgcccaagaccatccagatggttcgaagccagagaagcggcatggtgcagacagaggcccagtaccggttcatctacatggccgtgcagcattacatcgaaaccctgcagcggcggatcgaggaagaacagaagtccaagagaaagggccacgagtacaccaacatcaagtacagcctggccgaccagaccagcggcgatcaatctcctctgcctccttgcacacccacacctccatgtgccgagatgcgggaagatagcgccagggtgtacgagaacgtgggcctgatgcaacagcagaagtccttccgg

SEQ ID NO: 20 - DNA, 2124 bp, Artificial Sequence
  Description of Artificial Sequence Synthetic polynucleotide
tacaagatctacgacctgcacaagaagcggagctgcaatctggacgagcagcaagaactggtggaacgggacgacgagaagcagctgatgaacgtggaacccatccacgccgacatcctgctggaaacctacaagcggaagatcgccgacgagggcagactgttcctggccgagtttcagagcatccccagagtgttcagcaagttccccatcaaagaggccagaaagcccttcaaccagaacaagaaccgctacgtggacattctgccctacgactacaatcgcgtggaactgagcgagatcaatggcgacgccggcagcaactacatcaacgccagctacatcgacggcttcaaagagccccggaagtatatcgccgctcagggccctagagatgagacagtggacgacttctggcgcatgatttgggagcagaaagccaccgtgatcgtgatggttaccagatgcgaagagggcaacagaaacaagtgcgccgagtactggcccagcatggaagaaggcacaagagcctttggcgacgtggtggtcaagatcaatcagcacaagcggtgccccgactacatcatccagaaactgaacatcgtgaacaagaaagagaaggccaccggacgggaagtgacccacatccagtttaccagctggcccgatcatggcgtgcccgaggatccacatctgctgctcaagctgcggagaagagtgaacgccttcagcaacttcttcagcggccccatcgtggtgcactgttctgcaggcgttggaagaaccggcacctacatcggaatcgacgccatgctggaaggactggaagccgagaacaaggtggacgtgtacggctacgtggtcaagctgagaaggcagcggtgtctgatggtgcaggttgaggcccagtacatcctgatccatcaggccctggtcgagtacaaccagttcggcgagacagaagtgaacctgagcgagctgcacccctatctgcacaacatgaagaagcgggaccctccaagcgagccctctccactggaagctgagttccagagactgcccagctacagaagctggcggacacagcacatcggcaatcaagaggaaaacaagagcaagaaccggaacagcaacgtgatcccgtacgattacaacagagtgcccctgaagcacgaactcgagatgagcaaagagagcgagcacgacagcgacgagtccagcgacgatgatagcgacagcgaggaacccagcaagtatatcaatgcctccttcatcatgagctattggaagcccgaagtgatgattgccgcacagggacccctgaaagagacaatcggcgacttttggcagatgatcttccagcggaaagtgaaagtgatcgtcatgctgaccgagctgaaacacggcgaccaagagatctgcgcccagtattggggagagggaaagcagacctacggcgacattgaggtggacctgaaggacaccgacaagagcagcacctacacactgcgggtgttcgagctgagacactccaagagaaaggacagccggaccgtgtaccagtaccagtataccaattggagcgtggaacagctgcctgccgagcctaaagaactgatcagcatgatccaggtcgtgaagcagaagctgcctcagaagaacagcagcgagggaaacaagcaccacaagtctacccctctgctgatccactgcagagatggctctcagcagaccggcatcttctgcgccctgctgaatctcctggaaagcgccgagacagaggaagtggtggacatcttccaggtggtcaaagccctgcggaaggccagacctggcatggtgtctaccttcgagcagtatcagttcctgtacgacgtgatcgccagcacataccccgctcagaacggccaagtgaagaagaacaaccaccaagaggacaagatcgagttcgacaacgaggtggacaaagtgaagcaggacgccaactgcgtgaaccctctgggagccccagaaaagctgcctgaggccaaagaacaggccgagggctctgagccaacatctggaacagagggacctgagcacagcgtgaacggacctgctagccccgctctgaatcagggctct

SEQ ID NO: 21 - DNA, 651 bp, Artificial Sequence
  Description of Artificial Sequence Synthetic polynucleotide
atggaagccattgccaaatacgacttcaaggccaccgccgacgacgagctgagcttcaagagaggcgacatcctgaaggtgctgaacgaggaatgcgaccagaactggtacaaggccgagctgaacggcaaggacggcttcatccccaagaactacatcgagatgaagccccatccatggttcttcggcaagatccccagagccaaggccgaagagatgctgagcaagcagagacacgacggcgcctttctgatccgggaatctgaatctgcccctggcgacttcagcctgtccgtgaagttcggcaacgacgtgcagcacttcaaggtcctgagagatggcgccggaaagtacttcctgtgggtcgtgaagtttaacagcctgaacgagctggtggactaccacagatctaccagcgtgtcccggaaccagcagatcttcctgcgggacatcgaacaggttccccagcaacctacctacgtgcaggccctgttcgacttcgaccctcaagaggatggcgagctgggctttagacggggcgatttcatccacgtgatggacaatagcgaccccaactggtggaagggcgcctgtcatggacagaccggcatgttccccagaaactacgtgacccctgtgaaccggaacgtg

SEQ ID NO: 22 - DNA, 1209 bp, Artificial Sequence
  Description of Artificial Sequence Synthetic polynucleotide
atgacagccatcatcaaagaaatcgtgtcccggaacaagcggcgctaccaagaggatggcttcgacctggacctgacctacatctaccccaacatcattgccatgggcttccccgccgaaagactggaaggcgtgtacagaaacaacatcgacgatgtcgtgcggttcctggacagcaagcacaagaaccactacaagatctacaacctgtgcgccgagcggcactacgataccgccaagttcaactgcagagtggctcagtaccccttcgaggaccacaatcctccacagctggaactgatcaagcccttctgcgaggacctggatcagtggctgagcgaggacgataatcacgtggccgccattcactgcaaggccggcaagggaagaaccggcgtgatgatctgtgcctacctgctgcaccggggcaagtttctgaaagcccaagaggccctggacttctacggcgaagtgcggaccagagacaagaaaggcgtgacaatccccagccagcggagatacgtgtactactacagctatctgctgaagaaccacctggactacagacccgtggcactgctgttccacaagatgatgttcgagactatccccatgttcagcggcggcacatgcaaccctcagttcgtcgtgtgccagctgaaagtgaagatctactccagcaacagcggccccaccagacgcgaggacaagttcatgtacttcgagttccctcagcctctgcctgtgtgcggcgacatcaaggtggaattcttccacaagcagaacaagatgctgaaaaaggacaagatgttccacttctgggtcaacaccttcttcatccccggacctgaagagacaagcgagaaggtggaaaacggcagcctgtgcgaccaagagatcgacagcatctgcagcatcgagcgggccgacaacgacaaagaatacctggtgctgaccctgaccaagaacgatctggacaaggccaacaaggataaggccaaccggtacttcagccccaacttcaaagtgaaactgtacttcaccaagaccgtcgaggaacccagcaatcctgaggccagctctagcacatctgtgacccctgacgtgtccgacaatgagcccgaccactacagatacagcgacaccaccgacagcgaccccgagaacgagcctttcgatgaggatcagcacacacagatcaccaaagtg

SEQ ID NO: 23 - DNA, 2778 bp, Artificial Sequence
  Description of Artificial Sequence Synthetic polynucleotide
atgacaagcagattcagactgccagccggcagaacctacaatgtgcgggcttctgagctggccagagacagacagcacaccgaggtcgtgtgcaacatcctgctgctcgacaataccgtgcaggccttcaaagtgaacaagcacgaccagggccaagtcctgctggacgtggtgttcaagcacctggatctgaccgagcaggactacttcggactgcagctggccgacgacagcaccgacaatcctagatggctggaccccaacaagcccatccggaagcagctgaagagaggctccccttactctctgaacttccgggtcaagttcttcgtgtccgatcctaacaaactgcaagaagagtatacccgctaccagtacttcctgcagatcaagcaggacatcctgaccggcagactgccctgtccttctaatactgccgctctgctggcctcctttgccgtgcaatctgaactgggcgactacgaccagagcgagaacctgagcggctacctgagcgattacagcttcatccccaaccagcctcaggacttcgagaaagagatcgccaagctgcatcagcagcacatcggactgtctccagccgaggccgagttcaactacctgaacaccgccaggacactggaactgtacggcgtggaatttcactacgcccgggaccagagcaacaacgagatcatgattggcgtgatgagcggcggcatcctgatctacaagaacagagtgcggatgaacacattcccctggctgaagatcgtgaagatcagcttcaagtgcaagcagttcttcatccagctgcggaaagagctgcacgagagcagagagacactgctgggcttcaacatggtcaactaccgggcctgcaagaacctgtggaaggcctgtgtggaacaccacacctttttccggctggaccgacctctgcctcctcagaagaatttcttcgcccactacttcaccctgggcagcaagttcagatactgcggcagaacagaggtgcagtccgtgcagtacggcaaagagaaagccaacaaggaccgggtgttcgccagatctcctagcaagccactggccagaaagctgatggactgggaagtcgtgtcccggaacagcatcagcgacgacagactggaaacccagagcctgcctagcagaagccctcctggcacacccaaccacagaaacagcaccttcacacaagagggcacaagactgaggcctagctctgtgggacacctggtggatcacatggtgcacacaagccccagcgaggtgttcgtgaaccagagaagccctagctctacccaggccaacagcatcgtgctggaaagcagccccagccaagaaacaccaggcgacggaaaacctcctgctctgccacctaagcagagcaagaagaacagctggaaccagatccactacagccacagccagcaggatctggaaagccacatcaacgagacattcgacatccctagcagccccgagaagcccacacctaatggcggaatccctcacgacaacctggtgctgatccggatgaagcccgacgagaatggcagattcggcttcaacgtgaaaggcggctacgatcagaaaatgcccgtgatcgtgtccagagtggcccctggaactcctgccgatctgtgtgtgcccagactgaacgaaggcgaccaggtcgtgctgatcaacggcagagatatcgccgagcacacccacgatcaggtggtgctgttcattaaggcctcttgcgagagacacagcggcgaactgatgctgctcgtgcggcctaatgccgtgtacgacgtggtggaagagaaactggaaaacgagcccgacttccagtacatccctgagaaggccccactggacagcgtgcaccaggatgatcatagcctgcgcgagagcatgatccagctggcagagggactgatcaccggcacagtgctgacccagttcgaccagctgtaccggaagaaacctggcatgaccatgtcctgcgccaaactgcctcagaacatcagcaagaaccggtacagagacatcagcccctacgatgccaccagagtgatcctgaagggcaacgaggactacatcaacgccaattacatcaacatggaaatccccagctccagcatcatcaaccagtatatcgcctgtcagggcccactgcctcacacctgtaccgacttttggcagatgacctgggagcagggcagcagcatggtggtcatgctgacaacccaggtggaacggggcagagtgaagtgccaccagtattggcctgagcctaccggcagcagctcctacggctgttaccaagtgacctgccacagcgaagagggcaacaccgcctacatcttcagaaagatgaccctgttcaatcaagagaagaacgagagccggcctctgacacagatccagtatattgcttggcccgaccacggcgtgcccgacgatagttctgacttcctggacttcgtgtgccacgtgcgcaacaaacgcgccggaaaagaggaacctgtcgtcgtccactgtagcgccggcattggaagaaccggcgtgctgattaccatggaaacagccatgtgcctgatcgagtgcaatcagcccgtgtatcccctggacatcgtgcggaccatgagagatcagcgggccatgatgatccagacacctagccagtacagattcgtgtgcgaggccattctgaaggtgtacgaagagggattcgtgaagcccctgaccacctccaccaacaag

SEQ ID NO: 24 - DNA, 2418 bp, Artificial Sequence
  Description of Artificial Sequence Synthetic polynucleotide
gatcagagagagatcctgcagaagttcctggacgaggcccagagcaagaagatcaccaaagaggaattcgccaacgagttcctgaaactgaagcggcagagcaccaagtacaaggccgacaagacataccccaccaccgtggccgagaagcccaagaacatcaagaagaaccggtacaaggacatcctgccatacgactactccagagtggaactgagcctgatcaccagcgacgaggacagcagctacatcaacgccaacttcatcaagggcgtgtacggccccaaggcctatatcgcaacacagggccctctgtctacaaccctgctggacttctggcgcatgatctgggagtacagcgtgctgatcatcgtgatggcctgcatggaatacgagatgggcaagaagaagtgcgagcggtactgggccgaacctggcgaaatgcagctggaattcggccccttttccgtgtcctgcgaagccgagaagagaaagtccgactacatcatcaggaccctgaaagtgaagttcaacagcgaaacccggaccatctaccagtttcactacaagaactggcccgaccacgacgtgccaagcagcatcgatcctatcctggaactgatttgggacgtgcggtgctaccaagaggacgacagcgtgccaatctgcatccactgttctgccggctgcggaagaacaggcgtcatctgcgccatcgactacacctggatgctgctgaaggacggcatcatccccgagaacttcagcgtgttcagcctgatccgcgagatgagaacccagaggcctagcctggtgcagacccaagagcagtacgaactggtgtacaacgccgtgctggaactgttcaagagacagatggacgtgatccgggacaagcacagcggcacagagtctcaggccaaacactgcatccctgagaagaatcacaccctgcaggccgacagctacagccccaatctgcctaagagcaccaccaaggccgccaaaatgatgaaccagcagcggacaaagatggaaatcaaagagagcagctccttcgacttccggaccagcgagatcagcgccaaagaagaactggttctgcaccccgccaagtcctctaccagcttcgactttctcgagctgaactacagcttcgataagaacgccgacaccaccatgaagtggcagaccaaggcctttcctatcgtgggcgagcctctgcagaaacaccagagcctggatctgggctccctgctgtttgagggctgcagcaatagcaagcccgtgaacgccgctggccggtactttaatagcaaggtgcccatcaccaggaccaagagcacccctttcgagctgatccagcagcgcgagacaaaagaggtggacagcaaagagaacttctcctacctggaaagccagcctcacgacagctgcttcgtggaaatgcaggcccagaaagtgatgcacgtgtccagcgccgagctgaattactctctgccctacgacagcaagcaccagatccggaacgccagcaacgtgaagcaccacgatagctctgccctgggagtgtacagctacattcccctggtggaaaacccctacttcagctcctggccacctagcggcacaagcagcaagatgtctctggatctgcccgagaagcaggacggcacagtgttcccatctagcctgctgcctaccagcagcaccagcctgttcagctactacaatagccacgactctctgtccctgaacagccccaccaacatctccagcctgctgaatcaagaaagcgctgtgctggccaccgctccaagaatcgacgatgagatccctcctcctctgcctgtgcggacccctgagtctttcatcgtggtggaagaggccggcgagttcagccctaatgtgcccaaatctctgagcagcgccgtgaaagtcaagatcggcacctctctggaatggggcggcacatccgagcctaagaaattcgacgactccgtgatcctgaggccaagcaagagcgtgaagctgagaagccccaagtccgagctgcatcaggacagatctagccctcctccaccactgcctgagagaaccctcgagtcattcttcctggccgacgaggattgcatgcaggcacagagcatcgagacatacagcacaagctaccccgacaccatggaaaacagcacctccagcaagcagacactgaaaaccccaggcaagagcttcacccggtccaagagcctgaagatcctgcggaacatgaagaagtccatctgcaacagctgccctccaaacaagcctgccgagagcgtgcagtccaacaatagcagcagcttcctgaacttcggctttgccaaccggttcagcaagcctaagggccccagaaatcctcctcctacatggaacatc

SEQ ID NO: 25 - DNA, 762 bp, Artificial Sequence
  Description of Artificial Sequence Synthetic polynucleotide
atgcctgatcctgctgctcatctgccattcttctacggcagcatcagcagagccgaggccgaagaacatctgaagctggccggaatggccgacggactgtttctgctcagacagtgcctgagaagcctcggcggctatgtgctgtctctggtgcacgatgtgcggttccatcacttccccatcgagagacagctgaacggcacctacgctatcgctggcggaaaagcccattgtggacctgccgagctgtgcgagttctacagcagagatcctgacggcctgccttgcaacctgcggaagccttgcaatagacccagcggcctggaacctcagcctggcgttttcgactgcctgagagatgccatggtccgagattacgtgcggcagacctggaagctggaaggcgaagctctggaacaggcaatcatcagccaggctcctcaggtggaaaagctgatcgccacaacagcccacgagcggatgccttggtatcacagctccctgaccagagaggaagccgagcggaagctgtattctggcgcccagaccgatggcaagttcctgctgaggcccagaaaagagcagggcacatacgccctgagcctgatctacggcaagaccgtgtaccactacctgatctcccaggacaaggccggcaagtactgtatccctgagggcaccaagttcgacaccctgtggcagctggtggaatacctgaagctgaaagccgatggactgatctactgcctgaaagaggcctgc

SEQ ID NO: 26 - DNA, 1011 bp, Artificial Sequence
  Description of Artificial Sequence Synthetic polynucleotide
atgcctgatcctgctgctcatctgccattcttctacggcagcatcagcagagccgaggccgaagaacatctgaagctggccggaatggccgacggactgtttctgctcagacagtgcctgagaagcctcggcggctatgtgctgtctctggtgcacgatgtgcggttccatcacttccccatcgagagacagctgaacggcacctacgctatcgctggcggaaaagcccattgtggacctgccgagctgtgcgagttctacagcagagatcctgacggcctgccttgcaacctgcggaagccttgcaatagacccagcggcctggaacctcagcctggcgttttcgactgcctgagagatgccatggtccgagattacgtgcggcagacctggaagctggaaggcgaagctctggaacaggcaatcatcagccaggctcctcaggtggaaaagctgatcgccacaacagcccacgagcggatgccttggtatcacagctccctgaccagagaggaagccgagcggaagctgtattctggcgcccagaccgatggcaagttcctgctgaggcccagaaaagagcagggcacatacgccctgagcctgatctacggcaagaccgtgtaccactacctgatctcccaggacaaggccggcaagtactgtatccctgagggcaccaagttcgacaccctgtggcagctggtggaatacctgaagctgaaagccgatggactgatctactgcctgaaagaggcctgtcctaacagcagcgccagcaatgctagcggagctgctgcacctacactgcctgctcaccctagcacactgacacaccctcagcggagaatcgataccctgaacagcgacggctacaccccagaacctgccagaatcactagccccgacaagcccagacctatgcctatggacacctccgtgtacgagagcccctacagcgatcccgaggaactgaaggacaagaagctgttcctcaagcgggacaacctg

SEQ ID NO: 27 - DNA, 1857 bp, Artificial Sequence
  Description of Artificial Sequence Synthetic polynucleotide
atgcctgatcctgctgctcatctgccattcttctacggcagcatcagcagagccgaggccgaagaacatctgaagctggccggaatggccgacggactgtttctgctcagacagtgcctgagaagcctcggcggctatgtgctgtctctggtgcacgatgtgcggttccatcacttccccatcgagagacagctgaacggcacctacgctatcgctggcggaaaagcccattgtggacctgccgagctgtgcgagttctacagcagagatcctgacggcctgccttgcaacctgcggaagccttgcaatagacccagcggcctggaacctcagcctggcgttttcgactgcctgagagatgccatggtccgagattacgtgcggcagacctggaagctggaaggcgaagctctggaacaggcaatcatcagccaggctcctcaggtggaaaagctgatcgccacaacagcccacgagcggatgccttggtatcacagctccctgaccagagaggaagccgagcggaagctgtattctggcgcccagaccgatggcaagttcctgctgaggcccagaaaagagcagggcacatacgccctgagcctgatctacggcaagaccgtgtaccactacctgatctcccaggacaaggccggcaagtactgtatccctgagggcaccaagttcgacaccctgtggcagctggtggaatacctgaagctgaaagccgatggactgatctactgcctgaaagaggcctgtcctaacagcagcgccagcaatgctagcggagctgctgcacctacactgcctgctcaccctagcacactgacacaccctcagcggagaatcgataccctgaacagcgacggctacaccccagaacctgccagaatcactagccccgacaagcccagacctatgcctatggacacctccgtgtacgagagcccctacagcgatcccgaggaactgaaggacaagaagctgttcctcaagcgggacaacctgctgattgccgacatcgaactcggctgcggcaattttggatctgtgcggcagggcgtgtaccggatgcggaagaaacagatcgacgtggccatcaaggtgctgaagcagggaaccgagaaggccgacaccgaggaaatgatgcgggaagcccagattatgcaccagctggacaacccctacatcgtgcggctgatcggagtgtgtcaagccgaggctctgatgctggtcatggaaatggcaggcggaggccctctgcacaagtttctcgttggcaagcgggaagagatccccgtgtctaatgtggccgagctgctccaccaagtgtctatgggcatgaagtacctggaagagaagaacttcgtgcaccgcgacctggccgccagaaatgtgctgctggtcaacagacactacgccaagatcagcgacttcggcctgtctaaagccctgggcgccgacgatagcttcttcacagctagaagcgccggaaagtggcccctgaagtggtacgcccctgagtgcatcaacttccgcaagttcagctccagatccgacgtgtggtcttacggcgtgaccatgtgggaagccctgagctacggccagaaaccttacaagaagatgaagggccccgaagtcatggccttcatcgaacagggcaagagaatggaatgccctcctgagtgccctccagagctgtatgccctgatgagcgattgctggatctacaagtgggaagatcggcccgacttcctgaccgtggaacagagaatgcgggcctgctactactccctggcctccaaagttgagggacctcctggcagcacacagaaagccgaagctgcttgtgct

SEQ ID NO: 28 - DNA, 951 bp, Artificial Sequence
  Description of Artificial Sequence Synthetic polynucleotide
gatatgatcaccatcttcatcggcacctggaatatgggcaacgcccctccacctaagaagatcacaagctggtttctgagcaaaggccagggaaagaccagggacgacagcgccgattacatccctcacgatatctacgtgatcgggacccaagaggaccctctgagcgagaaagagtggctggaaattctgaagcactccctgcaagagatcacctccgtgaccttcaagaccgtggccatccacacactgtggaacatccggatcgtggtgctggccaagcctgagcacgagaacagaatcagccacatctgcaccgacaacgtgaaaaccggaatcgccaacacactgggcaacaaaggcgccgtgggagtgtccttcatgttcaacggcaccagcctgggcttcgtgaacagccacctgacatccggctccgagaagaagctgcggcggaaccagaactatatgaacatcctgcggtttctggccctgggcgacaagaagctgagccccttcaacatcacccaccggttcacccacctgttttggttcggcgacctgaactacagagtggacctgcctacctgggaagccgagacaatcatccagaagatcaagcagcaacagtacgccgacctgctgtcccacgatcagctgctgaccgagagaagggaacagaaagtgttcctgcacttcgaggaagaggaaatcacattcgcccctacctacagattcgagcggctgacccgggataagtacgcctacaccaagcagaaagccaccggcatgaagtacaacctgccttcttggtgcgaccgggtgctgtggaagtcttaccctctggtgcacgtcgtgtgccagtcttacggcagcaccagcgacatcatgaccagcgatcacagccctgtgttcgccacatttgaggccggcgtgaccagccagtttgtgtcc

SEQ ID NO: 29 - protein, 3 aa, Artificial Sequence
  Description of Artificial Sequence Synthetic peptide
GGS

SEQ ID NO: 30 - protein, 6 aa, Artificial Sequence
  Description of Artificial Sequence Synthetic peptide
GGSGGS

SEQ ID NO: 31 - protein, 9 aa, Artificial Sequence
  Description of Artificial Sequence Synthetic peptide
GGSGGSGGS

SEQ ID NO: 32 - protein, 12 aa, Artificial Sequence
  Description of Artificial Sequence Synthetic peptide
GGSGGSGGSGGS

SEQ ID NO: 33 - protein, 15 aa, Artificial Sequence
  Description of Artificial Sequence Synthetic peptide
GGSGGSGGSGGSGGS

SEQ ID NO: 34 - protein, 4 aa, Artificial Sequence
  Description of Artificial Sequence Synthetic peptide
GGGS

SEQ ID NO: 35 - protein, 8 aa, Artificial Sequence
  Description of Artificial Sequence Synthetic peptide
GGGSGGGS

SEQ ID NO: 36 - protein, 12 aa, Artificial Sequence
  Description of Artificial Sequence Synthetic peptide
GGGSGGGSGGGS

SEQ ID NO: 37 - protein, 16 aa, Artificial Sequence
  Description of Artificial Sequence Synthetic peptide
GGGSGGGSGGGSGGGS

SEQ ID NO: 38 - protein, 20 aa, Artificial Sequence
  Description of Artificial Sequence Synthetic peptide
GGGSGGGSGGGSGGGSGGGS

SEQ ID NO: 39 - protein, 5 aa, Artificial Sequence
  Description of Artificial Sequence Synthetic peptide
GGGGS

SEQ ID NO: 40 - protein, 10 aa, Artificial Sequence
  Description of Artificial Sequence Synthetic peptide
GGGGSGGGGS

SEQ ID NO: 41 - protein, 15 aa, Artificial Sequence
  Description of Artificial Sequence Synthetic peptide
GGGGSGGGGSGGGGS

SEQ ID NO: 42 - protein, 20 aa, Artificial Sequence
  Description of Artificial Sequence Synthetic peptide
GGGGSGGGGSGGGGSGGGGS

SEQ ID NO: 43 - protein, 25 aa, Artificial Sequence
  Description of Artificial Sequence Synthetic peptide
GGGGSGGGGSGGGGSGGGGSGGGGS

SEQ ID NO: 44 - protein, 18 aa, Artificial Sequence
  Description of Artificial Sequence Synthetic peptide
GSTSGSGKPGSGEGSTKG

SEQ ID NO: 45 - protein, 20 aa, Artificial Sequence
  Description of Artificial Sequence Synthetic peptide
EAAAKEAAAKEAAAKEAAAK

SEQ ID NO: 46 - protein, 42 aa, Artificial Sequence
  Description of Artificial Sequence Synthetic polypeptide
AAAIEVMYPPPYLDNEKSNGTIIHVKGKHLCPSPLFPGPSKP

SEQ ID NO: 47 - protein, 12 aa, Artificial Sequence
  Description of Artificial Sequence Synthetic peptide
ESKYGPPCPSCP

SEQ ID NO: 48 - protein, 12 aa, Artificial Sequence
  Description of Artificial Sequence Synthetic peptide
ESKYGPPAPSAP

SEQ ID NO: 49 - protein, 12 aa, Artificial Sequence
  Description of Artificial Sequence Synthetic peptide
ESKYGPPCPPCP

SEQ ID NO: 50 - protein, 12 aa, Artificial Sequence
  Description of Artificial Sequence Synthetic peptide
EPKSCDKTHTCP

SEQ ID NO: 51 - protein, 86 aa, Artificial Sequence
  Description of Artificial Sequence Synthetic polypeptide
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 52 - protein, 45 aa, Artificial Sequence
  Description of Artificial Sequence Synthetic polypeptide
TTTPAPRPPTPAPTIALQPLSLRPEACRPAAGGAVHTRGLDFACD

SEQ ID NO: 53 - protein, 132 aa, Artificial Sequence
  Description of Artificial Sequence Synthetic polypeptide
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 54 - protein, 34 aa, Artificial Sequence
  Description of Artificial Sequence Synthetic polypeptide
ACPTGLYTHSGECCKACNLGEGVAQPCGANQTVC

SEQ ID NO: 55 - protein, 20 aa, Artificial Sequence
  Description of Artificial Sequence Synthetic peptide
AVGQDTQEVIVVPHSLPFKV

SEQ ID NO: 56 - DNA, 126 bp, Artificial Sequence
  Description of Artificial Sequence Synthetic polynucleotide
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 57 - DNA, 36 bp, Artificial Sequence
  Description of Artificial Sequence Synthetic oligonucleotide
gaaagcaagtacggtccaccttgccctagctgtccg

SEQ ID NO: 58 - DNA, 36 bp, Artificial Sequence
  Description of Artificial Sequence Synthetic oligonucleotide
gaatccaagtacggccccccagcgcctagtgcccca

SEQ ID NO: 59 - DNA, 36 bp, Artificial Sequence
  Description of Artificial Sequence Synthetic oligonucleotide
gaatctaaatatggcccgccatgcccgccttgccca

SEQ ID NO: 60 - DNA, 36 bp, Artificial Sequence
  Description of Artificial Sequence Synthetic oligonucleotide
gaaccgaagtcttgtgataaaactcatacgtgcccg

SEQ ID NO: 61 - DNA, 258 bp, Artificial Sequence
  Description of Artificial Sequence Synthetic polynucleotide
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 62 - DNA, 396 bp, Artificial Sequence
  Description of Artificial Sequence Synthetic polynucleotide
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 63 - DNA, 102 bp, Artificial Sequence
  Description of Artificial Sequence Synthetic polynucleotide
gcctgccctacaggactctacacgcatagcggtgagtgttgtaaagcatgcaacctcggggaaggtgtagcccagccatgcggggctaaccaaaccgtttgc

SEQ ID NO: 64 - DNA, 60 bp, Artificial Sequence
  Description of Artificial Sequence Synthetic oligonucleotide
gctgtgggccaggacacgcaggaggtcatcgtggtgccacactccttgccctttaaggtg

SEQ ID NO: 65 - protein, 27 aa, Artificial Sequence
  Description of Artificial Sequence Synthetic peptide
FWVLVVVGGVLACYSLLVTVAFIIFWV

SEQ ID NO: 66 - protein, 46 aa, Artificial Sequence
  Description of Artificial Sequence Synthetic polypeptide
GPAGSLLGSGQMQITLWGSLAAVAIFFVITFLIFLCSSCDREKKPR

SEQ ID NO: 67 - protein, 36 aa, Artificial Sequence
  Description of Artificial Sequence Synthetic polypeptide
MEADALSPVGLGLLLLPFLVTLLAALCVRCRELPVS

SEQ ID NO: 68 - protein, 36 aa, Artificial Sequence
  Description of Artificial Sequence Synthetic polypeptide
MEADALSPVGLGLLLLPFLVTLLAALAVRARELPVS

SEQ ID NO: 69 - protein, 32 aa, Artificial Sequence
  Description of Artificial Sequence Synthetic polypeptide
EEAILVPCVLGLLLLPILAMLMALCVHCHRLP

SEQ ID NO: 70 - protein, 21 aa, Artificial Sequence
  Description of Artificial Sequence Synthetic peptide
IFSGFAGLLAILLVVAVFCIL

SEQ ID NO: 71 - protein, 21 aa, Artificial Sequence
  Description of Artificial Sequence Synthetic peptide
LCYLLDGILFIYGVILTALFL

SEQ ID NO: 72 - protein, 21 aa, Artificial Sequence
  Description of Artificial Sequence Synthetic peptide
ALIAFLAFLIIVTSIALLVVL

SEQ ID NO: 73 - DNA, 81 bp, Artificial Sequence
  Description of Artificial Sequence Synthetic oligonucleotide
ttttgggtgctggtggtggttggtggagtcctggcttgctatagcttgctagtaacagtggcctttattattttctgggtg

SEQ ID NO: 74 - DNA, 138 bp, Artificial Sequence
  Description of Artificial Sequence Synthetic polynucleotide
ggtccggctggctctctgctcggcagtggtcagatgcagattacgttgtggggcagtttggcagccgtcgcaatcttctttgttatcacttttcttatctttctctgttcctcatgtgacagagagaaaaagccccga

SEQ ID NO: 75 - DNA, 108 bp, Artificial Sequence
  Description of Artificial Sequence Synthetic polynucleotide
atggaagccgatgctctgtctcctgttggcctgggactgctgctcctgccttttctggttacactgctggccgctctgtgtgtgcggtgtagagaactgccagttagt

SEQ ID NO: 76 - DNA, 108 bp, Artificial Sequence
  Description of Artificial Sequence Synthetic polynucleotide
atggaagccgatgctctgtctcctgttggcctgggactgctgctcctgccttttctcgttacactgctggccgctctggctgtgcgagctagagaactgcctgtgtct

SEQ ID NO: 77 - DNA, 96 bp, Artificial Sequence
  Description of Artificial Sequence Synthetic oligonucleotide
gaggaagcaatcctggtgccgtgtgtacttggtctgcttttgttgccaatacttgcgatgctcatggctctctgcgtacattgccatcggcttccg

SEQ ID NO: 78 - DNA, 63 bp, Artificial Sequence
  Description of Artificial Sequence Synthetic oligonucleotide
atcttcagcggctttgccggactgctggctatcctgctggttgtggccgtgttctgtatcctt

SEQ ID NO: 79 - DNA, 63 bp, Artificial Sequence
  Description of Artificial Sequence Synthetic oligonucleotide
ctgtgctacctgctggacggcatcctgtttatctacggcgtgatcctgacagccctgttcctt

SEQ ID NO: 80 - DNA, 63 bp, Artificial Sequence
  Description of Artificial Sequence Synthetic oligonucleotide
gccctgattgccttcctggcctttctgatcatcgtgaccagcattgccctgctggtcgtgctg

SEQ ID NO: 81 - protein, 21 aa, Artificial Sequence
  Description of Artificial Sequence Synthetic peptide
MALPVTALLLPLALLLHAARP

SEQ ID NO: 82 - protein, 24 aa, Artificial Sequence
  Description of Artificial Sequence Synthetic peptide
METDTLLLWVLLLWVPGSTGAGGS

SEQ ID NO: 83 - protein, 22 aa, Artificial Sequence
  Description of Artificial Sequence Synthetic peptide
MLLLVTSLLLCELPHPAFLLIP

SEQ ID NO: 84 - protein, 242 aa, Artificial Sequence
  Description of Artificial Sequence Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 85 - protein, 504 aa, Artificial Sequence
  Description of Artificial Sequence Synthetic polypeptide
MALPVTALLLPLALLLHAARPQVQLVQSGAEVKKPGASVKVSCKASGYTFTNYWMHWVRQAPGQGLEWMGFITPTTGYPEYNQKFKDRVTMTADKSTSTAYMELSSLRSEDTAVYYCARRKVGKGVYYALDYWGQGTTVTVSSGGGGSGGGGSGGGGSDIQMTQSPSSLSASVGDRVTITCRASGNIHNYLAWYQQKPGKVPKLLIYNTKTLADGVPSRFSGSGSGTDYTLTISSLQPEDVATYYCQHFWSSPWTFGGGTKVEIKEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 817 aa, Artificial Sequence
  Description of Artificial Sequence Synthetic polypeptide
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDGPAGSLLGSGQMQITLWGSLAAVAIFFVITFLIFLCSSCDREKKPRSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHL

SEQ ID NO: 87 - protein, 1015 aa, Artificial Sequence
  Description of Artificial Sequence Synthetic polypeptide
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDFWVLVVVGGVLACYSLLVTVAFIIFWVSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHLEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 88 - protein, 946 aa, Artificial Sequence
  Description of Artificial Sequence Synthetic polypeptide
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDFWVLVVVGGVLACYSLLVTVAFIIFWVVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHLEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 89 - protein, 1020 aa, Artificial Sequence
  Description of Artificial Sequence Synthetic polypeptide
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDEEAILVPCVLGLLLLPILAMLMALCVHCHRLPSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHLEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 90 - protein, 1034 aa, Artificial Sequence
  Description of Artificial Sequence Synthetic polypeptide
METDTLLLWVLLLWVPGSTGAGGSDYKDDDDKGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITTTTPAPRPPTPAPTIALQPLSLRPEACRPAAGGAVHTRGLDFACDGPAGSLLGSGQMQITLWGSLAAVAIFFVITFLIFLCSSCDREKKPRSAIQAAWPSGTECIAKYNFHGTAEQDLPFCKGDVLTIVAVTKDPNWYKAKNKVGREGIIPANYVQKREGVKAGTKLSLMPWFHGKITREQAERLLYPPETGLFLVRESTNYPGDYTLCVSCDGKVEHYRIMYHASKLSIDEEVYFENLMQLVEHYTSDADGLCTRLIKPKVMEGTVAAQDEFYRSGWALNMKELKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELHLEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA